SEQUENCE LISTING (5 sequences)

SEQ ID NO: 1 - protein, 567 aa, synthetic construct
  source 1..567: note = Prokaryotic PAL from Anabaena variabilis
MKTLSQAQSKTSSQQFSFTGNSSANVIIGNQKLTINDVARVARNGTLVSLTNNTDILQGIQASCDYINNAVESGEPIYGVTSGFGGMANVAISREQASELQTNLVWFLKTGAGNKLPLADVRAAMLLRANSHMRGASGIRLELIKRMEIFLNAGVTPYVYEFGSIGASGDLVPLSYITGSLIGLDPSFKVDFNGKEMDAPTALRQLNLSPLTLLPKEGLAMMNGTSVMTGIAANCVYDTQILTAIAMGVHALDIQALNGTNQSFHPFIHNSKPHPGQLWAADQMISLLANSQLVRDELDGKHDYRDHELIQDRYSLRCLPQYLGPIVDGISQIAKQIEIEINSVTDNPLIDVDNQASYHGGNFLGQYVGMGMDHLRYYIGLLAKHLDVQIALLASPEFSNGLPPSLLGNRERKVNMGLKGLQICGNSIMPLLTFYGNSIADRFPTHAEQFNQNINSQGYTSATLARRSVDIFQNYVAIALMFGVQAVDLRTYKKTGHYDARACLSPATERLYSAVRHVVGQKPTSDRPYIWNDNEQGLDEHIARISADIAAGGVIVQAVQDILPCLH

SEQ ID NO: 2 - protein, 567 aa, synthetic construct
  source 1..567: note = Exemplary AvPAL subunit
MKTLSQAQSKTSSQQFSFTGNSSANVIIGNQKLTINDVARVARNGTLVSLTNNTDILQGIQASCDYINNAVESGEPIYGVTSGFGGMANVAISREQASELQTNLVWFLKTGAGNKLPLADVRAAMLLRANSHMRGASGIRLELIKRMEIFLNAGVTPYVYEFGSIGASGDLVPLSYITGSLIGLDPSFKVDFNGKEMDAPTALRQLNLSPLTLLPKEGLAMMNGTSVMTGIAANCVYDTQILTAIAMGVHALDIQALNGTNQSFHPFIHNSKPHPGQLWAADQMISLLANSQLVRDELDGKHDYRDHELIQDRYSLRCLPQYLGPIVDGISQIAKQIEIEINSVTDNPLIDVDNQASYHGGNFLGQYVGMGMDHLRYYIGLLAKHLDVQIALLASPEFSNGLPPSLLGNRERKVNMGLKGLQICGNSIMPLLTFYGNSIADRFPTHAEQFNQNINSQGYTSATLARRSVDIFQNYVAIALMFGVQAVDLRTYKKTGHYDARASLSPATERLYSAVRHVVGQKPTSDRPYIWNDNEQGLDEHIARISADIAAGGVIVQAVQDILPCLH

SEQ ID NO: 3 - protein, 567 aa, synthetic construct
  source 1..567: note = Exemplary AvPAL subunit
MKTLSQAQSKTSSQQFSFTGNSSANVIIGNQKLTINDVARVARNGTLVSLTNNTDILQGIQASCDYINNAVESGEPIYGVTSGFGGMANVAISREQASELQTNLVWFLKTGAGNKLPLADVRAAMLLRANSHMRGASGIRLELIKRMEIFLNAGVTPYVYEFGSIGASGDLVPLSYITGSLIGLDPSFKVDFNGKEMDAPTALRQLNLSPLTLLPKEGLAMMNGTSVMTGIAANCVYDTQILTAIAMGVHALDIQALNGTNQSFHPFIHNSKPHPGQLWAADQMISLLANSQLVRDELDGKHDYRDHELIQDRYSLRCLPQYLGPIVDGISQIAKQIEIEINSVTDNPLIDVDNQASYHGGNFLGQYVGMGMDHLRYYIGLLAKHLDVQIALLASPEFSNGLPPSLLGNRERKVNMGLKGLQICGNSIMPLLTFYGNSIADRFPTHAEQFNQNINSQGYTSATLARRSVDIFQNYVAIALMFGVQAVDLRTYKKTGHYDARACLSPATERLYSAVRHVVGQKPTSDRPYIWNDNEQGLDEHIARISADIAAGGVIVQAVQDILPSLH

SEQ ID NO: 4 - protein, 567 aa, synthetic construct
  source 1..567: note = Exemplary AvPAL subunit
MKTLSQAQSKTSSQQFSFTGNSSANVIIGNQKLTINDVARVARNGTLVSLTNNTDILQGIQASCDYINNAVESGEPIYGVTSGFGGMANVAISREQASELQTNLVWFLKTGAGNKLPLADVRAAMLLRANSHMRGASGIRLELIKRMEIFLNAGVTPYVYEFGSIGASGDLVPLSYITGSLIGLDPSFKVDFNGKEMDAPTALRQLNLSPLTLLPKEGLAMMNGTSVMTGIAANCVYDTQILTAIAMGVHALDIQALNGTNQSFHPFIHNSKPHPGQLWAADQMISLLANSQLVRDELDGKHDYRDHELIQDRYSLRCLPQYLGPIVDGISQIAKQIEIEINSVTDNPLIDVDNQASYHGGNFLGQYVGMGMDHLRYYIGLLAKHLDVQIALLASPEFSNGLPPSLLGNRERKVNMGLKGLQICGNSIMPLLTFYGNSIADRFPTHAEQFNQNINSQGYTSATLARRSVDIFQNYVAIALMFGVQAVDLRTYKKTGHYDARASLSPATERLYSAVRHVVGQKPTSDRPYIWNDNEQGLDEHIARISADIAAGGVIVQAVQDILPSLH

SEQ ID NO: 5 - protein, 4 aa, synthetic construct
  source 1..4: note = Exemplary peptide cleavable spacer
GGFG

(19) United States
(12) Patent Application Publication
(10) Pub. No.: US 2025/0161466 A1
(43) Pub. Date: May 22, 2025

(54) CONJUGATES OF PHENYLALANINE AMMONIA-LYASE AND POEGMA AND METHODS FOR THEIR USE

(71) Applicant: BIOMARIN PHARMACEUTICAL INC., Novato, CA (US)

(72) Inventors: Debobrato DAS, Novato, CA (US); Calvin Erik ABEL, San Francisco, CA (US); Geoffrey BERGUIG, Corte Madera, CA (US); Benjamin SPANGLER, Richmond, CA (US)

(73) Assignee: BIOMARIN PHARMACEUTICAL INC., Novato, CA (US)

(21) Appl. No.: 18/948,893

(22) Filed: Nov. 15, 2024

Related U.S. Application Data
(60) Provisional application No. 63/685,484, filed on Aug. 21, 2024.
(60) Provisional application No. 63/670,613, filed on Jul. 12, 2024.
(60) Provisional application No. 63/600,515, filed on Nov. 17, 2023.

(51) Int. Cl. A61K 47/60; A61K 9/00; A61K 38/51; A61P 3/00

(52) U.S. Cl. CPC A61K 47/60; A61K 9/0019; A61K 38/51; A61P 3/00; C12Y 403/01005

(57) ABSTRACT

The present disclosure provides conjugates of phenylalanine ammonia-lyase and poly(oligo(ethylene glycol)methacrylate (POEGMA) and methods for their use. In particular, the POEGMA-PAL conjugates disclosed herein are immune-masked for a reduced immune response upon administration to a subject.

### CROSS-REFERENCE TO RELATED APPLICATIONS

This application claims the benefit of U.S. Provisional Patent Application No. 63/685,484, filed Aug. 21, 2024, U.S. Provisional Patent Application No. 63/670,613, filed Jul. 12, 2024 and U.S. Provisional Patent Application No. 63/600,515, filed Nov. 17, 2023, the disclosure of each of which is incorporated by reference herein in its entirety.

### SEQUENCE LISTING

This application contains an electronic Sequence Listing which has been submitted in XML file format with this application, the entire content of which is incorporated by reference herein in its entirety. The Sequence Listing XML file submitted with this application is entitled “11808-545-999_SEQ LISTING.xml”, was created on Nov. 8, 2024, and is 8,430 bytes in size.

### 1. FIELD

The present disclosure provides conjugates of phenylalanine ammonia-lyase and poly(oligo(ethylene glycol)methacrylate) (POEGMA) and methods for their therapeutic use. In particular, the POEGMA-PAL conjugates disclosed herein provide a method for treating phenylketonuria (PKU) having reduced immunogenicity compared to current methods for treating PKU.

### 2. BACKGROUND

Phenylalanine hydroxylase (PAH) deficiency, commonly also referred to as phenylketonuria (PKU), is a rare autosomal recessive genetic disorder associated with absent or deficient PAH activity. PAH deficiency prevents the body from processing phenylalanine, which is an essential amino acid found in all protein-containing foods. This deficiency is caused by a lack of activity in the hepatic enzyme PAH, which converts phenylalanine to tyrosine. Without this enzyme, phenylalanine levels in the blood rise and can cause neurocognitive and psychiatric symptoms in a subject having the disorder. Additionally, the excretion of excessive phenylalanine and its metabolites can create a musty body odor and skin conditions, such as eczema, and associated inhibition of tyrosinase and low tyrosine levels can lead to decreased skin and hair pigmentation.

PAH deficiency results in intolerance to the dietary intake of the essential amino acid phenylalanine and thus, lifelong dietary management to sustain phenylalanine levels below recommended thresholds is important. Without effective therapy, most individuals with PKU develop profound and irreversible intellectual disability. Pegvaliase (e.g., PALYNZIQ®) is a pegylated phenylalanine ammonia-lyase (PAL) enzyme substitution therapy approved to lower phenylalanine concentration in adults with PKU who have uncontrolled phenylalanine concentration >600 μmol/L on existing management.

PAL is a non-mammalian enzyme widely distributed in plants (Koukol, et al., J. Biol. Chem. 236:2692-2698 (1961); Hanson, et al., The Enzymes 7:75-166 (1972); Poppe, et al., Curr. Org. Chem. 7:1297-1315 (2003)), some fungi (Rao, et al., Can. J. Biochem. 4512:1863-1872 (1967); Abell, et al., Methods Enzymol. 142:242-253 (1987)) and bacteria (Bezanson, et al., Can. J. Microbiol. 16:147-151 (1970); Xiang, et al., J. Biol. Chem. 277:32505-32509 (2002); Hill, et al., Chem. Commun. 1358-1359 (2003)) and can be recombinantly produced in Escherichia coli. PAL from cyanobacteria species Anabaena variabilis (Av) has been cloned and expressed in bacteria and displays PAL enzyme activity in vitro and in vivo (see, e.g., U.S. Pat. Nos. 7,531,341; 7,534,595; 7,537,923; and 7,560,263, each of which are incorporated by reference herein in its entirety). Pegvaliase comprises recombinant Anabaena variabilis PAL (rAvPAL) derivatized by covalent attachment of polyethylene glycol (PEG), which reduced its immunogenicity, improved its half-life, and optimized its pharmacokinetic profile (Id.) compared to PEG-based therapies available before its approval. However, Pegvaliase still elicits some immune response and use of Pegvaliase is associated with a greater than 30% discontinuation rate.

Accordingly, there is an opportunity for further development of PAL-based therapies for subjects with PKU. In other words, there is still the need to provide a further improved PKU treatment having reduced immunogenicity, improved half-life, and/or an optimized pharmacokinetic profile. The conjugates and methods disclosed herein provide an alternative polymer that mimics PEG's physicochemical properties without compromising therapeutic pharmacokinetics and minimizes the anti-PEG immune responses that limits the efficacy of previous PEGylated drugs, such as Pegvaliase.

### 3. SUMMARY

In one aspect, the present disclosure provides conjugates of phenylalanine ammonia-lyase (PAL) and poly(oligo(ethylene glycol)methacrylate)) (POEGMA) (poly-oligoethylene glycol methacrylate (POEGMA)-modified recombinant bacterial phenylalanine ammonia lyase; herein “POEGMA-PAL conjugates”) and methods for their therapeutic use. The POEGMA-PAL conjugates disclosed herein provide a delivery system for PAL that a) elicits a lower immunogenetic response to anti-PEG antibodies and b) displays faster pharmacokinetics than current PAL-based therapies. Additionally, POEGMA-PAL conjugates can be formulated as a low viscosity solution for subcutaneous delivery of higher doses than available for current PAL-based therapies.

In one embodiment, the present disclosure provides a conjugate “POEGMA-PAL conjugate” comprising one or more polymers (e.g., 1-21) comprising poly(oligoethylene glycol methacrylate); and phenylalanine ammonia lyase (PAL), wherein the one or more polymers is directly or indirectly bound to the PAL via one or more lysine residues.

In one embodiment, the POEGMA-PAL conjugate of the present disclosure is represented by Formula (I):

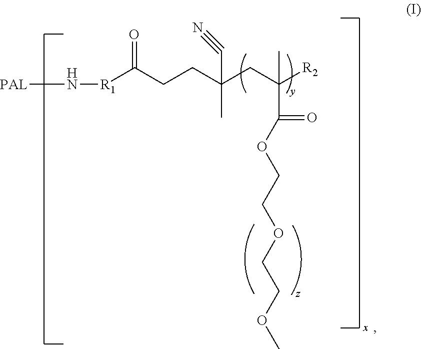

wherein:
PAL is a prokaryotic phenylalanine ammonia-lyase;
R₁ is a linker;
R₂ is an end cap;
x is an integer from 1 to about 21;
y is an integer from about 50 to about 500; and
z is an integer from 2 to about 10, and
N in “NH” is the nitrogen of the side chain of a lysine residue of PAL.

In Formula (I), the one or more polymers comprising poly(oligoethylene glycol methacrylate) can be represented by Formula (V):

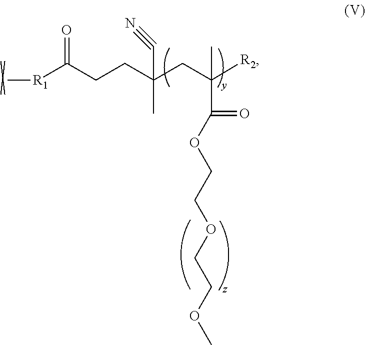

wherein: [symbol]
is the attachment to the nitrogen (N) directly bound to PAL in Formula (I);
R₁ is a linker;
R₂ is an end cap;
y is an integer from about 50 to about 500; and
z is an integer from 2 to about 10.

In one embodiment, 19-21 of the lysine residues of PAL are conjugated to a linker-POEGMA. In one embodiment, at least 4 lysine residues on each subunit of PAL are conjugated to a linker-POEGMA. In one embodiment, at least 5 lysine residues on each subunit of PAL are conjugated to a linker-POEGMA. In one embodiment, 4-6 lysine residues on each subunit of PAL are conjugated to a linker-POEGMA. Each linker-POEGMA may be the same or may be different.

In one embodiment, the POEGMA-PAL conjugate is represented by Formula (I′):

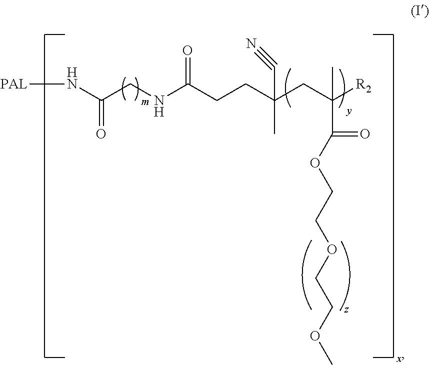

wherein:
PAL is a prokaryotic phenylalanine ammonia-lyase, which is a homotetrameric protein comprising four subunits;
R₂ is an end cap;
m is an integer from about 3 to about 12;
x is an integer from 1 to about 21;
y is an integer from about 50 to about 500; and
z is an integer from 2 to about 10.

In one embodiment, the POEGMA-PAL conjugate is represented by Formula (Ia):

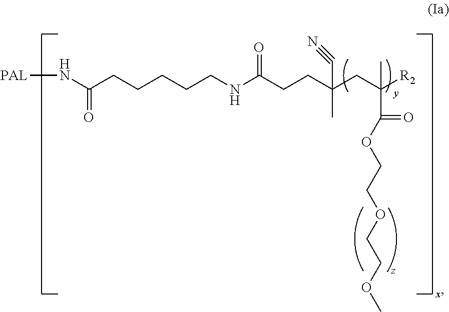

wherein:
PAL is a prokaryotic phenylalanine ammonia-lyase;
R₂ is an end cap;
x is an integer from 1 to about 21;
y is an integer from about 50 to about 500; and
z is an integer from 2 to about 10.

In one embodiment, the POEGMA-PAL conjugate is represented by Formula (Ia-1):

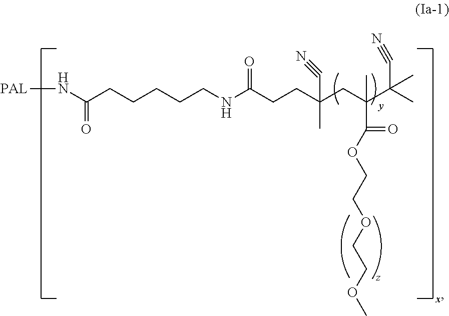

wherein:
PAL is a prokaryotic phenylalanine ammonia-lyase;
x is an integer from 1 to about 21;
y is an integer from about 50 to about 500; and
z is an integer from 2 to about 10.

In one embodiment, the POEGMA-PAL conjugate is represented by Formula (Ia-1-1):

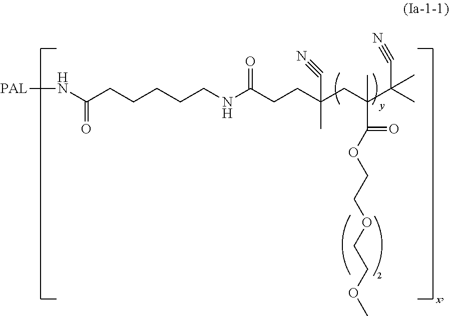

wherein:
PAL is a prokaryotic phenylalanine ammonia-lyase, which is a homotetrameric protein comprising four subunits;
x is an integer from 1 to about 21; and
y is an integer from about 50 to about 500.

In one embodiment, the POEGMA-PAL conjugate is as depicted in Table A.

In certain embodiments of each of Formulas (I), (I′), (Ia), (Ia-1), (Ia-1-1), or any of (Ia-1-11)-(Ia-1-46), PAL may be any prokaryotic phenylalanine ammonia-lyase. In some embodiments, PAL is derived from Anabaena variabilis (AvPAL). In one embodiment, each subunit of AvPAL comprises an amino acid sequence of SEQ ID NO:1. In one embodiment, the AvPAL is a recombinant AvPAL (rAvPAL). In one embodiment, the AvPAL is an AvPAL variant. In one embodiment, each subunit of the AvPAL variant comprises an amino acid sequence of SEQ ID NO:2, SEQ ID NO:3 or SEQ ID NO:4.

In some embodiments of Formula (I), (I′), (Ia), (Ia-1), and (Ia-1-1), and any of Formulas (Ia-1-11)-(Ia-1-46), a polymer comprising poly(oligoethylene glycol methacrylate) is bound to one or more (e.g., 1-5 or 1-6) lysine residues on each subunit of PAL, wherein the lysine residues of each subunit of PAL comprise independently one or more of K189, K195, K522, K413, K145, K32, K115, K335, and K109. In some embodiments of Formula (I), (I′), (Ia), (Ia-1), and (Ia-1-1), or any of Formula (Ia-1-11)-(Ia-1-46), a polymer comprising poly(oligoethylene glycol methacrylate) is bound to two or more lysine residues on each subunit of PAL (e.g., 2-5 or 2-6) residues on each subunit of PAL), wherein the lysine residues of each subunit of PAL comprise independently two or more of K189, K195, K522, K413, K145, K32, K115, K335, and K109. In some embodiments of Formulas (I), (I′), (Ia), (Ia-1), and (Ia-1-1), a polymer comprising poly(oligoethylene glycol methacrylate) is bound to three or more lysine residues on each subunit of PAL (e.g., 3-5 or 3-6) residues on each subunit of PAL), wherein the lysine residues of each subunit of PAL comprise independently three or more of K189, K195, K522, K413, K145, K32, K115, K335, and K109. In some embodiments of Formulas (I), (I′), (Ia), (Ia-1), and (Ia-1-1), or any of Formula (Ia-1-11)-(Ia-1-46), a polymer comprising poly(oligoethylene glycol methacrylate) is bound to four or more lysine residues on each subunit of PAL (e.g., 4-5 or 4-6), wherein the lysine residues of each subunit of PAL comprise independently four or more of K189, K195, K522, K413, K145, K32, K115, K335, and K109. In some embodiments of Formula (I), (I′), (Ia), (Ia-1), and (Ia-1-1), or any of Formulas (Ia-1-11)-(Ia-1-46), a polymer comprising poly(oligoethylene glycol methacrylate) is bound to five or more lysine residues on each subunit of PAL (e.g., 5 or 6 residues), wherein the lysine residues of each subunit of PAL comprise independently four or more of K189, K195, K522, K413, K145, K32, K115, K335, and K109.

In some embodiments, three of the one or more polymers comprising poly(oligoethylene glycol methacrylate) are individually bound to each of lysine residues K189, K195 and K522 on each subunit. In some embodiments, five polymers of the one or more polymers comprising poly(oligoethylene glycol methacrylate) are individually bound to each of lysine residues K189, K195, K522, K413, and K145 on each subunit.

In certain embodiments of Formulas (I), (I′), (Ia), (Ia-1), and (Ia-1-1), or any of Formula (Ia-1-11)-(Ia-1-46), R₁ is CH₂, C(O)—C₁₋₁₂ alkyl-NH, C(O)—(O—CH₂—CH₂) n, wherein n is 2 or 3, or Gly-Gly-Phe-Gly (SEQ ID NO:5). In one embodiment, R₁ is C(O)—(CH₂)₅—NH.

In certain embodiments of Formulas (I), (I′), (Ia), (Ia-1), and (Ia-1-1), or any of Formula (Ia-1-11)-(Ia-1-46), R₂ is isobutyronitrile, 4-cyanovaleric acid, 4-methoxy-2,4-dimethylvaleronitrile, or —OH (hydroxy). In one embodiment, R₂ is isobutyronitrile.

In certain embodiments of Formulas (I), (I′), (Ia), and (Ia-1), z is 2 to about 8. In one embodiment, z is 2. In one embodiment, x is an integer from about 5 to about 21, about 8 to about 21, about 10 to about 21, about 16 to about 21, or about 19 to about 21. In one embodiment, x is 19. In one embodiment, x is 20. In one embodiment x is 21. In one embodiment, y is an integer from about 150 to about 300, such as about 175 to 200. In one embodiment, y is about 200.

In another aspect, the present disclosure provides a composition comprising a plurality of POEGMA-PAL conjugates of Formula (I), (I′), (Ia), (Ia-1), or (Ia-1-1), or any of Formula (Ia-1-11)-(Ia-1-46). In such a composition, x, y, and z may vary from conjugate to conjugate and within a single conjugate in the composition. As such, a composition comprising a plurality of POEGMA-PAL conjugates may be characterized by a zₐᵥₑ of about 2 to about 8, an xₐᵥₑ of about 8 to about 21 or about 16 to about 21 and a yₐᵥₑ of about 150 to about 300, such as about 175 to about 200. In some embodiments, zₐᵥₑ is 2. In some embodiments, the standard deviation of zₐᵥₑ is 0 (i.e., z is 2 in all POEGMA-PAL conjugates of the composition). In some embodiments, a composition comprising a plurality of POEGMA-PAL conjugates may be characterized by a number average molecular weight (Mₙ) of about 1100 kDa to about 1300 kDa. In some embodiments, a composition comprising a plurality of POEGMA-PAL conjugates may be characterized by a number average molecular weight (Mₙ) of about 1200 kDa. In some embodiments, a composition comprising a plurality of POEGMA-PAL conjugates may be characterized by a number average molecular weight (M_{w}) of about 1100 kDa to about 1300 kDa. In some embodiments, a composition comprising a plurality of POEGMA-PAL conjugates may be characterized by a number average molecular weight (M_{w}) of about 1200 kDa. In some embodiments, a composition comprising a plurality of POEGMA-PAL conjugates may be characterized by a polydispersity index (PI) of about 1 to about 1.1.

In another aspect, the present disclosure provides a pharmaceutical composition comprising a plurality of POEGMA-PAL conjugates of Formula (I), (I′), (Ia), (Ia-1), (Ia-1-1), and/or any of Formulas (Ia-1-11)-(Ia-1-46) and one or more pharmaceutically acceptable excipients. In certain embodiments, a pharmaceutical composition comprises about 20 mg/mL to about 60 mg/mL of a plurality of POEGMA-PAL conjugates of Formula (I), (I′), (Ia), (Ia-1), (Ia-1-1), and/or any of Formulas (Ia-1-11)-(Ia-1-46) and one or more pharmaceutically acceptable excipients. In certain embodiments, a pharmaceutical composition comprises about 20 mg/mL to about 60 mg/mL of a plurality of POEGMA-PAL conjugates of Formula (I), (I′), (Ia), (Ia-1), (Ia-1-1), and/or any of Formulas (Ia-1-11)-(Ia-1-46) and one or more pharmaceutically acceptable excipients and has a viscosity of equal to or less than about 70 cP, such as a viscosity of about 1 cP to about 70 cP. In certain embodiments, a pharmaceutical composition comprises about 20 mg/mL to about 40 mg/mL of a plurality of POEGMA-PAL conjugates of Formula (I), (I′), (Ia), (Ia-1), (Ia-1-1), and/or any of Formulas (Ia-1-11)-(Ia-1-46) and one or more pharmaceutically acceptable excipients and has a viscosity of equal to or less than about 20 cP, such as a viscosity of about 1 cP to about 20 cP.

In yet another aspect, the present disclosure provides a method of treating PAH deficiency in a subject, the method comprising administering a therapeutically effective amount of POEGMA-PAL conjugate of any one of Formula (I), (I′), (Ia), (Ia-1), (Ia-1-1), and/or any of Formulas (Ia-1-11)-(Ia-1-46) to the subject.

In yet another aspect, the present disclosure provides a method of treating phenylketonuria (PKU) in a subject, the method comprising administering a therapeutically effective amount of POEGMA-PAL conjugate of any one of Formula (I), (I′), (Ia), (Ia-1), (Ia-1-1), and/or any of Formulas (Ia-1-11)-(Ia-1-46) to the subject.

In yet another aspect, the present disclosure provides a method of reducing blood phenylalanine concentration in a subject in need thereof, the method comprising administering a therapeutically effective amount of POEGMA-PAL conjugate of any one of Formulas (I), (I′), (Ia), (Ia-1), (Ia-1-1), and/or any of Formulas (Ia-1-11)-(Ia-1-46) to the subject.

In one embodiment, the POEGMA-PAL conjugate is administered in the form of a pharmaceutical composition comprising a plurality of POEGMA-PAL conjugates and one or more pharmaceutically acceptable excipients. In certain embodiments, the reducing of blood phenylalanine concentration in a subject is effective to treat PKU in the subject. In some embodiments, the administration is subcutaneous administration. In some embodiments, about 0.5 mL to about 1.5 mL of the pharmaceutical composition is administered to the subject via, e.g., subcutaneous injection.

In yet another aspect, the present disclosure provides a kit, wherein the kit comprises a syringe and a pharmaceutical composition comprising a plurality of POEGMA-PAL conjugates of any one of Formulas (I), (I′), (Ia), (Ia-1), (Ia-1-1), and/or any of Formulas (Ia-1-11)-(Ia-1-46) and one or more pharmaceutically acceptable excipients. In one embodiment, the syringe is filled with 0.5 to 1.5 mL of the pharmaceutical composition.

In yet another aspect, the present disclosure provides a method of making a POEGMA-PAL conjugate of Formula (I):

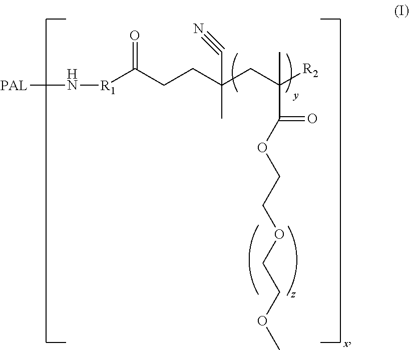

wherein:
PAL is a prokaryotic phenylalanine ammonia-lyase, which is a homotetrameric protein comprising four subunits;
R₁ is a linker;
R₂ is an end cap;
x is an integer from 1 to about 21;
y is an integer from about 50 to about 500; and
z is an integer from 2 to about 10,
   the method comprising:
a. contacting oligoethylene glycol methacrylate with a compound of Formula (II):

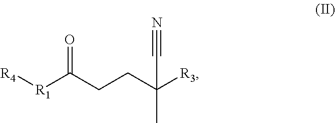

wherein R₃ is a RAFT chain transfer agent and R₄ is a conjugation moiety, under conditions effective to yield a polymer of Formula (III):

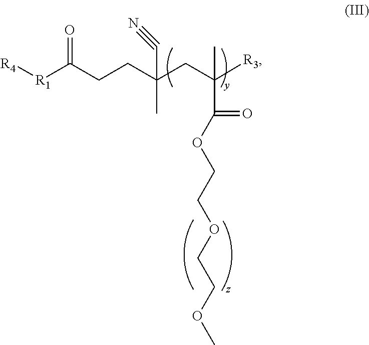

b. contacting the polymer of Formula (III) with an end group modification agent to remove the RAFT chain transfer agent and generate a pre-conjugation polymer of Formula (IV):

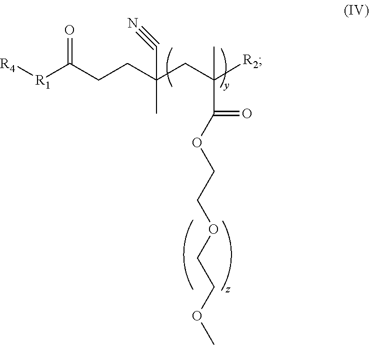

wherein R₂ is an end cap; and
c. conjugating the pre-conjugation polymer of Formula (IV) to a prokaryotic phenylalanine ammonia-lyase (PAL) variant via one or more lysine residues on the PAL variant (e.g., 1-21 residues).

In one embodiment, R₁ is CH₂, C(O)—C₁₋₁₂ alkyl-NH, C(O)—(O—CH₂—CH₂)ₙ, wherein n is 2 or 3, or Gly-Gly-Phe-Gly (SEQ ID NO:5). In one embodiment, R₁ is C(O)—C₁₋₁₂ alkyl-NH. In one embodiment, the conjugate of Formula (I) prepared as described herein is a conjugate of Formula (I′):

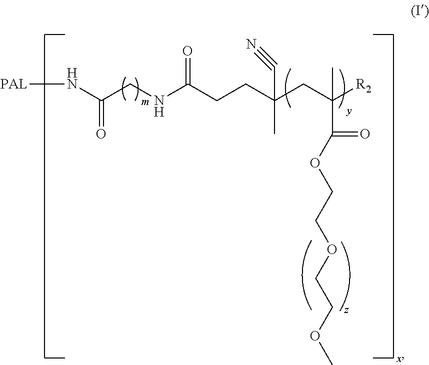

wherein:
PAL is a prokaryotic phenylalanine ammonia-lyase, which is a homotetrameric protein comprising four subunits;
R₂ is an end cap;
m is an integer from about 3 to about 12;
x is an integer from 1 to about 21;
y is an integer from about 50 to about 500; and
z is an integer from 2 to about 10.

In one embodiment, R₁ is C(O)—(CH₂)₅—NH. In one embodiment, the conjugate of Formula (I) prepared as described herein is a conjugate of Formula (Ia):

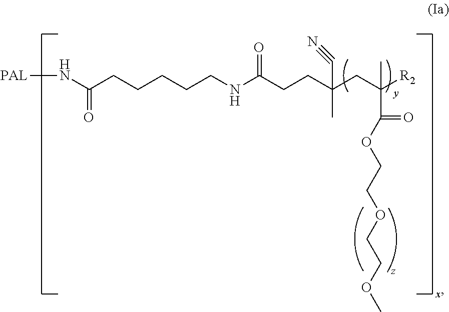

wherein:
PAL is a prokaryotic phenylalanine ammonia-lyase, which is a homotetrameric protein comprising four subunits;
R₂ is an end cap;
x is an integer from 1 to about 21;
y is an integer from about 50 to about 500; and
z is an integer from 2 to about 10.

In one embodiment, R₂ is isobutyronitrile, 4-cyanovaleric acid, 4-methoxy-2,4-dimethylvaleronitrile, and a hydroxy group (—OH). In one embodiment, R₂ is isobutyronitrile.

In one embodiment, the conjugate of Formula (I) prepared as described herein is a conjugate of Formula (Ia-1):

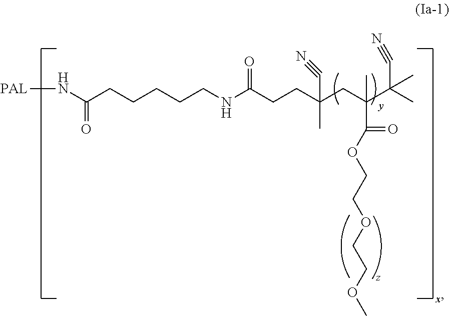

wherein:
PAL is a prokaryotic phenylalanine ammonia-lyase, which is a homotetrameric protein comprising four subunits;
x is an integer from 1 to about 21;
y is an integer from about 50 to about 500; and
z is an integer from 2 to about 10.

In one embodiment, z is 2 to about 8. In one embodiment, z is 2. In one embodiment, the conjugate of Formula (I) prepared as described herein is a conjugate of Formula (Ia-1-1):

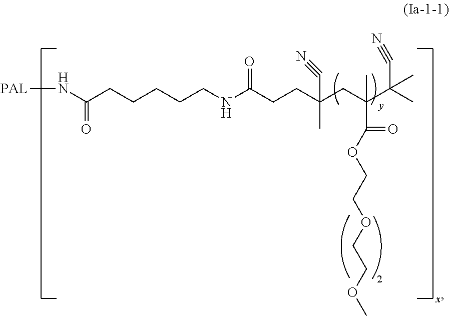

wherein:
PAL is a prokaryotic phenylalanine ammonia-lyase, which is a homotetrameric protein comprising four subunits;
x is an integer from 1 to about 21; and
y is an integer from about 50 to about 500.

In one embodiment, R₄ is

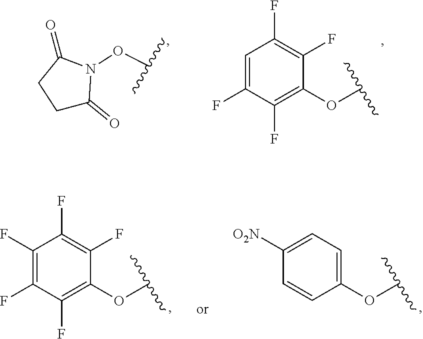

wherein [symbol] represents attachment to R₁.

In any of the Formulas disclosed herein depicting PAL, the PAL can be derived from Anabaena variabilis (AvPAL). In one embodiment, each subunit of the AvPAL comprises an amino acid sequence of SEQ ID NO:1. In one embodiment, the AvPAL is a recombinant AvPAL (rAvPAL). In one embodiment, the AvPAL is an AvPAL variant. In one embodiment, each subunit of the AvPAL variant comprises an amino acid sequence of SEQ ID NO: 2, SEQ ID NO:3 or SEQ ID NO:4.

In any embodiment, x can be an integer from about 8 to about 21, such as about 10 to about 21, about 16 to about 21 or about 19 to about 21. In any embodiment, e.g., in any of Formulas (I), (I′), (Ia), (Ia-1), and (Ia-1-1), y can be an integer from about 150 to about 300, such as about 175 to about 200.

In one embodiment, R₃ is a trithiocarbonate. In one embodiment, R₃ is

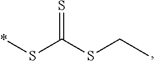 [SMILES: *SC(=S)SCC]

wherein the asterisk (*) represents attachment to the rest of compound or polymer.

In one embodiment, the conditions effective to generate the polymer of Formula (III) comprise contacting the oligoethylene glycol methacrylate with a compound of Formula (II) in the presence of DMSO and a light source. In one embodiment, the light source is blue LED light.

In one embodiment, the end group modification agent is azobisisobutyronitrile (AIBN), 4′-azobis(4-cyanovaleric acid), 2,2′-azobis(4-methoxy-2,4-dimethylvaleronitrile), or UV light. In one embodiment, the contacting of the polymer of Formula (III) with AIBN to generate a pre-conjugation polymer of Formula (IV) is in the presence of DMSO.

### 4. DRAWINGS

FIG. 1 depicts one embodiment of a POEGMA-PAL conjugate as disclosed herein.

FIG. 2 reports the viscosity of increasingly concentrated compositions of POEGMA-PAL as compared to PEG-PAL.

FIG. 3 reports the viscosity of Pegvaliase versus POEGMA-PAL.

FIG. 4 reports the viscosity of a 20 mg/mL, 40 mg/mL, and 60 mg/mL 45 kDa POEGMA-PAL solution compared to the viscosity Pegvaliase.

FIG. 5 depicts an exemplary experiment undertaken to determine cross-reactivity of POEGMA-PAL with anti-PEG IgG.

FIG. 6 reports the cross-reactivity of mouse monoclonal anti-PEG IgG with two different POEGMA polymers used in the POEGMA-PAL conjugates disclosed herein as compared to PEG-PAL.

FIG. 7 reports the cross-reactivity of PEG (top) and POEGMA-PAL (bottom) with existing anti-PEG IgG from Pegvaliase-treated subjects.

FIG. 8 reports the cross-reactivity of PEG and POEGMA-PAL with existing anti-PEG IgG from naïve subjects.

FIG. 9 reports plasma phenylalanine (Phe) plasma levels in male PAH^{enu2-/-} mice administered 45 kDa POEGMA-PAL as disclosed herein.

FIG. 10 reports plasma PAL plasma levels in male PAH^{enu2-/-} mice administered 45 kDa POEGMA-PAL as disclosed herein.

FIG. 11 reports plasma phenylalanine (Phe) in male PAH^{enu2-/-} mice administered 45 kDa POEGMA-PAL as disclosed herein.

FIG. 12 reports plasma PAL levels in male PAH^{enu2-/-} mice administered 45 kDa POEGMA-PAL as disclosed herein.

FIG. 13 reports the distribution of lysine residues on PAL that are conjugated to a POEGMA-linker in the POEGMA-PAL conjugates disclosed herein.

FIG. 14 reports plasma PAL and phenylalanine concentrations in C57BL6 PAH^{enu2} mice after subcutaneous or intravenous administration of 20 mg/kg 45 kDa POEGMA-PAL or Pegvaliase.

FIG. 15 reports plasma PAL and phenylalanine concentrations in cynomolgus monkeys after subcutaneous administration of 4 mg/kg 45 kDa POEGMA-PAL or Pegvaliase.

### 5. DETAILED DESCRIPTION

Described herein are conjugates of phenylalanine ammonia-lyase (PAL) and poly(oligo(ethylene glycol)methacrylate) (POEGMA), compositions and pharmaceutical compositions comprising the same, and methods of reducing blood phenylalanine concentration, which are effective to treat PKU, in a subject using the conjugates. The POEGMA-PAL conjugates described herein exhibit a reduced time to efficacy compared to current treatments for PKU (e.g., Pegvaliase or PALYNZIQ®). Further, compositions comprising high concentrations of POEGMA-PAL conjugates can be prepared at a low viscosity, providing a mechanism by which higher doses can be provided to a subject via injection, particularly via injection routes where volume is limited (e.g., subcutaneous, intramuscular). Additionally, the POEGMA-PAL conjugates disclosed herein display reduced antigenicity as compared to Pegvaliase. As such, a subject undergoing treatment with the POEGMA-PAL conjugates as disclosed herein may experience fewer adverse effects compared to previous treatment regimens and/or may be more effectively treated due to higher dosing and/or improved pharmacokinetics.

### Definitions

Unless otherwise stated, the following terms used in this application, including the specification and claims, have the definitions given below.

The singular forms “a,” “an” and “the” include plural referents unless the context clearly dictates otherwise.

All publications, patents and patent applications cited herein, whether supra or infra, are incorporated by reference in their entirety.

In some embodiments, chemical structures are disclosed with a corresponding chemical name. In case of conflict, the chemical structure controls the meaning, rather than the name.

The term “alkyl” refers to monovalent aliphatic hydrocarbyl groups that lack a double or triple bonds between carbon atoms, and that in certain embodiments, have from one to twelve carbon atoms (C₁₋₁₂ alkyl), such as one to 10, one to 6, one to 5, one to 4, or one to three carbon atoms. This term includes, by way of example, linear, branched, and cyclic hydrocarbyl groups such as methyl (—CH₃), ethyl (—CH₂CH₃), n-propyl (—CH₂CH₂CH₃), isopropyl (—CH(CH₃)₂), cyclopropyl (—C₃H₅), n-butyl (—CH₂CH₂CH₂CH₃), isobutyl (—CH₂CH(CH₃)₂), sec-butyl(CH(CH₃)(CH₃CH₂)), t-butyl (—C(CH₃)₃), n-pentyl (—CH₂CH₂CH₂CH₂CH₃), and neopentyl (—CH₂C(CH₃)₃).

In one embodiment, the term “alkyl” includes, by way of example, linear, branched, and cyclic hydrocarbyl groups such as methyl (—CH₃), ethyl (—CH₂CH₃), n-propyl (—CH₂CH₂CH₃), isopropyl (—CH(CH₃)₂), cyclopropyl (—C₃H₅), n-butyl (—CH₂CH₂CH₂CH₃), isobutyl (—CH₂CH(CH₃)₂), sec-butyl(CH(CH₃)(CH₃CH₂)), t-butyl (—C(CH₃)₃), n-pentyl (—CH₂CH₂CH₂CH₂CH₃), neopentyl (—CH₂C(CH₃)₃), hexyl (—CH₂ (CH₂)₄CH₃), heptyl (—CH₂ (CH₂)₅CH₃), octyl (—CH₂ (CH₂)₆CH₃), nonyl (—CH₂ (CH₂)₇CH₃), decyl (—CH₂ (CH₂)₈CH₃), undecyl (—CH₂ (CH₂)₉CH₃), and dodecyl (—CH₂ (CH₂)₁₀CH₃).

“Wild-type” (wt) is a term referring to the natural genetic form of an organism. A wild-type is distinguished from a mutant form (an organism with a genetic mutation).

As used herein, “polymer” refers to a series of monomer groups linked together. The POEGMA polymers described herein are prepared from m-PEGMA (poly(ethylene glycol) methyl ether methacrylate) monomers, as shown below:

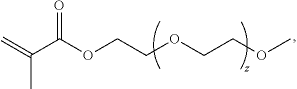

wherein z is an integer from 2 to about 10.

As used herein “POEGMA” refers to poly(oligoethylene glycol methacrylate) as shown below:

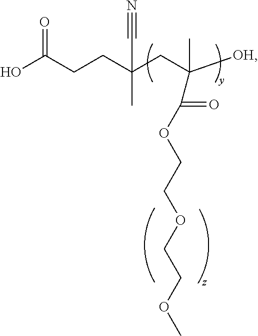

wherein y is an integer from about 50 to about 500 and z is an integer from 2 to about 10.

“Linker” refers to a chemical moiety that links two groups together (e.g., the nitrogen of the side chain of a lysine residue in PAL and POEGMA). The linker can be cleavable or non-cleavable. Cleavable linkers can be hydrolyzable, enzymatically cleavable, pH sensitive, photolabile, or disulfide linkers, fluorinated esters, phenyl esters, carbamates, carbonates, among others.

“Hydrolyzable linker” refers to a chemical linkage or bond, such as a covalent bond, which undergoes hydrolysis under physiological conditions. The tendency of a bond to hydrolyze may depend not only on the general type of linkage connecting two central atoms between which the bond is severed, but also on the substituents attached to these central atoms. Non-limiting examples of hydrolytically susceptible linkages include esters of carboxylic acids, phosphate esters, acetals, ketals, acyloxyalkyl ether, imines, orthoesters, and some amide linkages.

“Enzymatically cleavable linker” refers to a linkage that is subject to degradation by one or more enzymes. Some hydrolytically susceptible linkages may also be enzymatically degradable. For example, esterases may act on esters of carboxylic acid or phosphate esters, and proteases may act on peptide bonds and some amide linkages.

“pH sensitive linker” refers to a linkage that is stable at one pH and subject to degradation at another pH. For example, the pH sensitive linker can be stable at neutral or basic conditions, but labile at mildly acidic conditions.

“Photolabile linker” refers to a linkage, such as a covalent bond, which cleaves upon exposure to light. The photolabile linker includes an aromatic moiety to absorb the incoming light, which then triggers a rearrangement of the bonds to cleave the two groups linked by the photolabile linker.

The terms “polypeptide” and “protein” refer to a polymer of amino acid residues and are not limited to a minimum length of the product. Thus, peptides, oligopeptides, dimers, multimers, and the like, are included within the definition. Both full-length proteins and fragments thereof are encompassed by the definition. The terms also include post-expression modifications of the polypeptide, for example, glycosylation, acetylation, phosphorylation, and the like. Furthermore, “polypeptide” as used herein refers to a protein, which includes modifications, such as deletions, additions, and substitutions (generally conservative in nature), to the native sequence, as long as the protein maintains the desired activity. Such polypeptides may be referred to as “mutants” herein. These modifications can be deliberate, as through site-directed mutagenesis, or can be accidental, such as through mutations arising with hosts that produce the proteins or errors due to PCR amplification.

Conventional notation is used herein to portray polypeptide and protein (including enzyme) sequences: the left-hand end of a polypeptide sequence is the amino-terminus; the right-hand end of a polypeptide sequence is the carboxyl-terminus.

As used herein, “prokaryotic PAL” or “PAL” refers to (1) wild-type PAL from a prokaryotic organism, including, but not limited to PAL from Streptomyces maritimus, Nostoc punctiforme, Anabaena variabilis, Anacystis nidulans (Lofflehardt, Z. Naturforsch. 31 (11-12): 693-9 (1976), Photorabdus luminescens TT01 (Williams, et al., Microbiology 151:2543-2550 (2005), and Streptomyces verticillatus (Bezanson, et al., Can. J. Microbiol. 16 (3): 147-51 (1970); (2) fragments, mutants, variants or analogs of such wild-type PAL enzymes that retain similar (e.g., at least 50%) catalytic activity for phenylalanine, and that can, for example, exhibit increased catalytic activity, greater biochemical stability, increased half-life, and/or decreased immunogenicity, and (3) chemically modified versions of such wild-type PAL enzymes or fragments, mutants, variants or analogs thereof that are linked to other chemical moieties that provide other advantageous effects, such as, for example and not for limitation, enhanced half-life and/or decreased immunogenicity. For example, any references to methods of making or using prokaryotic PAL, and fragments, mutants, variants, analogs, or chemically modified versions thereof, and compositions of such enzyme(s), for therapeutic purposes, are meant to refer to methods of making, using, or formulating all such wild-type prokaryotic PAL or fragments, mutants, variants, analogs, or chemical modifications thereof. In any embodiment disclosed herein, PAL may be any prokaryotic phenylalanine ammonia-lyase, for example, any PAL disclosed in International Patent Publication Nos. WO 2008/153776 A1, WO 2011/097335 A2, and WO 2022/45924 A1, each which is incorporated herein by reference in its entirety. In one embodiment, PAL is a prokaryotic PAL from Anabaena variabilis (e.g., having a subunit amino acid sequence of SEQ ID NO:1) or biologically active fragment, mutant, variant or analog thereof:

(SEQ ID NO: 1)
MKTLSQAQSKTSSQQFSFTGNSSANVIIGNQKLTINDVARVARNGTLVS
LTNNTDILQGIQASCDYINNAVESGEPIYGVTSGFGGMANVAISREQAS
ELQTNLVWFLKTGAGNKLPLADVRAAMLLRANSHMRGASGIRLELIKRM
EIFLNAGVTPYVYEFGSIGASGDLVPLSYITGSLIGLDPSFKVDFNGKE
MDAPTALRQLNLSPLTLLPKEGLAMMNGTSVMTGIAANCVYDTQILTAI
AMGVHALDIQALNGTNQSFHPFIHNSKPHPGQLWAADQMISLLANSQLV
RDELDGKHDYRDHELIQDRYSLRCLPQYLGPIVDGISQIAKQIEIEINS
VTDNPLIDVDNQASYHGGNFLGQYVGMGMDHLRYYIGLLAKHLDVQIAL
LASPEFSNGLPPSLLGNRERKVNMGLKGLQICGNSIMPLLTFYGNSIAD
RFPTHAEQFNQNINSQGYTSATLARRSVDIFQNYVAIALMFGVQAVDLR
TYKKTGHYDARACLSPATERLYSAVRHVVGQKPTSDRPYIWNDNEQGLD
EHIARISADIAAGGVIVQAVQDILPCLH.

In certain embodiments, the POEGMA-PAL conjugate comprises a PAL variant exhibiting phenylalanine-converting activity and/or reduced immunogenicity as compared to a wild-type prokaryotic PAL. In certain embodiments, the POEGMA-PAL conjugate comprises a PAL variant with greater biochemical stability and/or biochemical half-life as compared to a wild-type prokaryotic PAL. In certain embodiments, the POEGMA-PAL conjugate comprises an Anabaena variabilis PAL (AvPAL) variant.

PAL (and variants thereof, such as AvPAL) is a homotetrameric protein with a molecular weight of 62 kDa per subunit. In certain embodiments, the POEGMA-PAL conjugate comprises an AvPAL variant wherein one or more amino acid residues on each subunit have been substituted by another amino acid residue. In some embodiments, one or more cysteine residues of the AvPAL variant have been substituted by a serine residue. In some embodiments, the one or more cysteine residues of the AvPAL variant that have been substituted by one or more serine residues are selected from the group consisting of cysteine residues at positions 503 and 565. In certain embodiments, the amino acid sequence of each AvPAL subunit is SEQ ID NO: 2, wherein the cysteine residue at position 503 has been substituted by a serine residue: MKTLSQAQSKTSSQQFSFTGNSSANVIIGNQKLTINDVARVARNGTLVSLTNNTDILQ GIQASCDYINNAVESGEPIYGVTSGFGGMANVAISREQASELQTNL VWFLKTGAGNK LPLADVRAAMLLRANSHMRGASGIRLELIKRMEIFLNAGVTPYVYEFGSIGASGDLV PLSYITGSLIGLDPSFKVDFNGKEMDAPTALRQLNLSPLTLLPKEGLAMMNGTSVMT GIAANCVYDTQILTAIAMGVHALDIQALNGTNQSFHPFIHNSKPHPGQLWAADQMIS LLANSQLVRDELDGKHDYRDHELIQDRYSLRCLPQYLGPIVDGISQIAKQIEIEINSVT DNPLIDVDNQASYHGGNFLGQYVGMGMDHLRYYIGLLAKHLDVQIALLASPEFSNG LPPSLLGNRERKVNMGLKGLQICGNSIMPLLTFYGNSIADRFPTHAEQFNQNINSQGY TSATLARRSVDIFQNYVAIALMFGVQAVDLRTYKKTGHYDARASLSPATERLYSAV RHVVGQKPTSDRPYIWNDNEQGLDEHIARISADIAAGGVIVQAVQDILPCLH (SEQ ID NO: 2), where cysteine residue at position 503 is bold and underlined.

In certain embodiments, the amino acid sequence of each AvPAL subunit is SEQ ID NO: 3, wherein the cysteine residue at position 565 has been substituted by a serine residue: MKTLSQAQSKTSSQQFSFTGNSSANVIIGNQKLTINDVARVARNGTLVSLTNNTDILQ GIQASCDYINNAVESGEPIYGVTSGFGGMANVAISREQASELQTNLVWFLKTGAGNK LPLADVRAAMLLRANSHMRGASGIRLELIKRMEIFLNAGVTPYVYEFGSIGASGDLV PLSYITGSLIGLDPSFKVDFNGKEMDAPTALRQLNLSPLTLLPKEGLAMMNGTSVMT GIAANCVYDTQILTAIAMGVHALDIQALNGTNQSFHPFIHNSKPHPGQLWAADQMIS LLANSQL VRDELDGKHDYRDHELIQDRYSLRCLPQYLGPIVDGISQIAKQIEIEINSVT DNPLIDVDNQASYHGGNFLGQYVGMGMDHLRYYIGLLAKHLDVQIALLASPEFSNG LPPSLLGNRERKVNMGLKGLQICGNSIMPLLTFYGNSIADRFPTHAEQFNQNINSQGY TSATLARRSVDIFQNYVAIALMFGVQAVDLRTYKKTGHYDARACLSPATERLYSAV RHVVGQKPTSDRPYIWNDNEQGLDEHIARISADIAAGGVIVQAVQDILPSLH (SEQ ID NO: 3), where cysteine residue at position 565 is bold and underlined.

In a certain embodiment, the amino acid sequence of each AvPAL subunit is SEQ ID NO: 4, where the cysteine residues at positions 503 and 565 have been substituted by serine residues: MKTLSQAQSKTSSQQFSFTGNSSANVIIGNQKLTINDVARVARNGTLVSLTNNTDILQ GIQASCDYINNAVESGEPIYGVTSGFGGMANVAISREQASELQTNLVWFLKTGAGNK LPLADVRAAMLLRANSHMRGASGIRLELIKRMEIFLNAGVTPYVYEFGSIGASGDLV PLSYITGSLIGLDPSFKVDFNGKEMDAPTALRQLNLSPLTLLPKEGLAMMNGTSVMT GIAANCVYDTQILTAIAMGVHALDIQALNGTNQSFHPFIHNSKPHPGQLWAADQMIS LLANSQLVRDELDGKHDYRDHELIQDRYSLRCLPQYLGPIVDGISQIAKQIEIEINSVT DNPLIDVDNQASYHGGNFLGQYVGMGMDHLRYYIGLLAKHLDVQIALLASPEFSNG LPPSLLGNRERKVNMGLKGLQICGNSIMPLLTFYGNSIADRFPTHAEQFNQNINSQGY TSATLARRSVDIFQNYVAIALMFGVQAVDLRTYKKTGHYDARASLSPATERLYSAV RHVVGQKPTSDRPYIWNDNEQGLDEHIARISADIAAGGVIVQAVQDILPSLH (SEQ ID NO: 4), where cysteine residues at positions 503 and 565 are bold and underlined.

PAL variants can also include fusion proteins in which the PAL enzyme has been fused to another heterologous polypeptide, such as a native or modified constant region of an immunoglobulin or a fragment thereof that retains the salvage epitope, known in the art to increase half-life.

As used herein, “variant,” “analog,” or “derivative” is a compound, e.g., a PAL enzyme, having more than about 70% sequence but less than 100% sequence similarity with a given compound, e.g., a peptide. Such variants, analogs or derivatives can comprise non-naturally occurring amino acid residues, including by way of example and not limitation, homoarginine, ornithine, penicillamine, and norvaline, as well as naturally occurring amino acid residues. Such variants, analogs or derivatives can also comprise one or a plurality of D-amino acid residues and can contain non-peptide interlinkages between two or more amino acid residues.

“Contacting” refers to the process of bringing into contact at least two distinct species such that they can react. It should be appreciated, however, that the resulting reaction product can be produced directly from a reaction between the added reagents or from an intermediate that can be produced in the reaction mixture.

“Treatment” or “treating” as used herein refers to prophylactic treatment or therapeutic treatment or diagnostic treatment. A “prophylactic” treatment is a treatment administered to a subject who does not exhibit signs of disease or pathology, or exhibits only early signs for the purpose of decreasing the risk of developing pathology. The pharmaceutical compositions comprising the POEGMA-PAL conjugates, as disclosed herein, can be given as a prophylactic treatment to reduce the likelihood of developing a pathology, e.g., PKU, or to minimize the severity of the pathology, if developed. A “therapeutic” treatment is a treatment administered to a subject who exhibits signs or symptoms of pathology for the purpose of diminishing or eliminating those signs or symptoms. The signs or symptoms can be biochemical, cellular, histological, functional, subjective, or objective. The POEGMA-PAL conjugate compositions can be given as a therapeutic treatment or for diagnosis.

As used herein, the terms “prevent,” “preventing,” and “prevention” refer to the total or partial inhibition of the development, recurrence, onset or spread of a disease and/or symptom related thereto (e.g., a disease or symptom related thereto that is associated with elevated phenylalanine levels, such as PKU, in a subject), resulting from the administration of a therapy or combination of therapies provided herein.

“Pharmaceutical composition” refers to a composition suitable for pharmaceutical use in subject animal, including humans and mammals.

“Pharmaceutically acceptable” and “pharmacologically acceptable” refer to a material that is not biologically or otherwise undesirable, i.e., the material can be administered to an individual without causing any undesirable biological effects or interacting in a deleterious manner with any of the components of the composition in which it is contained.

As used herein, the term “subject” and “patient” encompasses mammals and non-mammals. Examples of mammals include, but are not limited to, any member of the mammalian class: humans, non-human primates such as chimpanzees, and other apes and monkey species; farm animals such as cattle, horses, sheep, goats, swine; domestic animals such as rabbits, dogs, and cats; laboratory animals including rodents, such as rats, mice and guinea pigs, and the like. Examples of non-mammals include, but are not limited to, birds, fish, and the like. The term does not denote a particular age or gender. As used herein, the terms “subject” and “patient” are used interchangeably. As used herein, a subject is, in certain embodiments, a mammal such as a non-primate (e.g., cows, pigs, horses, cats, dogs, rats, etc.) or a primate (e.g., monkey or human), in certain embodiments, a human. In some embodiments, the subject is a mammal, and in certain embodiments, a human. In some embodiments, the subject has a disease or symptom related thereto that is associated with elevated phenylalanine levels, such as hyperphenylalaninemia (HPA) or PKU (e.g., classic PKU, moderate PKU, mild PKU or any subpopulation thereof). In some embodiments, the subject is a subject receiving enzyme substitution therapy or (“EST”, e.g., rAvPAL or rAvPAL-PEG) for elevated phenylalanine levels (e.g., a subject with PKU).

In some embodiments, the subject has a blood phenylalanine level of about 300 μmol/L to about 350 μmol/L. In some embodiments, the subject has a blood phenylalanine level of about 350 μmol/L to about 400 μmol/L In some embodiments, the subject has a blood phenylalanine level of about 400 μmol/L to about 450 μmol/L. In some embodiments, the subject has a blood phenylalanine level of about 450 μmol/L to about 500 μmol/L. In some embodiments, the subject has a blood phenylalanine level of about 500 μmol/L to about 550 μmol/L. In some embodiments, the subject has a blood phenylalanine level of about 550 μmol/L to about 600 μmol/L. In some embodiments, the subject has a blood phenylalanine level of greater than about 350 μmol/L. In some embodiments, the subject has a blood phenylalanine level of greater than about 360 μmol/L. In some embodiments, the subject has a blood phenylalanine level of greater than about 370 μmol/L. In some embodiments, the subject has a blood phenylalanine level of greater than about 380 μmol/L. In some embodiments, the subject has a blood phenylalanine level of greater than 390 μmol/L. In some embodiments, the subject has a blood phenylalanine level of greater than about 400 μmol/L. In some embodiments, the subject has a blood phenylalanine level of greater than about 600 μmol/L. As used herein, the term “therapeutically effective” refers to a dose or an amount of POEGMA-PAL conjugate that is effective to produce the intended beneficial effect on health of a subject to which the conjugate is administered. In some embodiments, a therapeutically effective amount of a POEGMA-PAL conjugate decrease L-phenylalanine (Phe) levels in the blood, plasma, or serum, in certain embodiments plasma, of the subject, thereby providing benefit to the subject. In some embodiments, a therapeutically effective amount of the POEGMA-PAL conjugate reduces plasma phenylalanine levels by greater than about 10%, 30%, 50%, 70%, 90%, 95%, 98% or 99% in a treated subject (e.g., a subject administered the POEGMA-PAL conjugate) when compared to an untreated subject. In some embodiments, a therapeutically effective amount of the POEGMA-PAL conjugate maintains plasma phenylalanine levels in static condition in a treated subject as compared to an untreated subject. In some embodiments, a therapeutically effective amount of POEGMA-PAL conjugate increases survival time or disease-free time by at least about 10%, 20%, 50%, 100%, 2-fold, 5-fold, or 10-fold longer in a treated subject compared to an untreated subject. The amount of POEGMA-PAL conjugate considered “therapeutically effective” will vary from one individual subject to another and will depend upon several factors, including the overall physical condition of the subject, diet, and disease state. A therapeutically effective amount of a pharmaceutical composition comprising the POEGMA-PAL conjugate as disclosed herein may be readily ascertained by one skilled in the art using publicly available materials and procedures.

### 5.1 POEGMA-PAL Conjugates

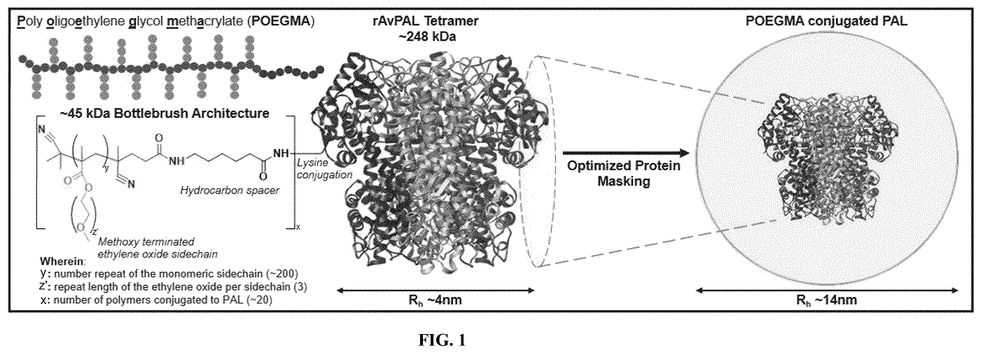

The POEGMA-PAL conjugates disclosed herein are a second-generation product to Pegvaliase and address the challenges associated with PEG as described in the Background Section. The POEGMA-PAL conjugates disclosed herein use poly-oligoethylene glycol methacrylate (POEGMA), which is a ˜45 KDa bottlebrush polymer consisting of about 200 repeats of short-tri-ethylene glycol methyl esters esterified to a synthetic methacrylate backbone to minimize anti-PEG antibody reactivity to ethylene oxide residues, as depicted in FIG. 1. The POEGMA-PAL conjugate disclosed herein use the protein/enzymatic component, rAvPAL (SEQ ID NO:4 as disclosed herein), which is responsible for reducing blood phenylalanine levels.

The POEGMA-PAL conjugates disclosed herein are modified PAL enzymes that convert phenylalanine to ammonia and trans-cinnamic acid. Without wishing to be bound by theory, it is believed that the POEGMA-PAL conjugates act as a substitute for the deficient PAH enzyme activity in subject with PAH deficiency, e.g., PKU subjects and HPA subjects, and reduce blood phenylalanine concentrations.

The POEGMA-PAL conjugates, as disclosed herein, unexpectedly exhibit improved immunogenicity and pharmacokinetics. Without wishing to be bound by theory, it is believed that the POEGMA-PAL conjugate structure minimizes the accessibility and number of solvent accessible antigenic repeats, to improve immunogenicity, bioavailability (and pharmacokinetic profile) and dosing formulation limitations encountered with products currently marketed for treating PKU and HPA (e.g., Pegvaliase). This, in turn, provides an enzyme substitution therapy with an improved dosing regimen, a shorter time to onset of action, an improved safety profile, and a reduced injection burden.

In one aspect, the present disclosure provides a POEGMA-PAL conjugate comprising:
a. a polymer comprising poly(oligoethylene glycol methacrylate) (POEGMA); and
b. phenylalanine ammonia lyase (PAL),
   wherein the polymer is directly or indirectly bound to the PAL.

In one embodiment, the POEGMA-PAL conjugate is represented by Formula (I):

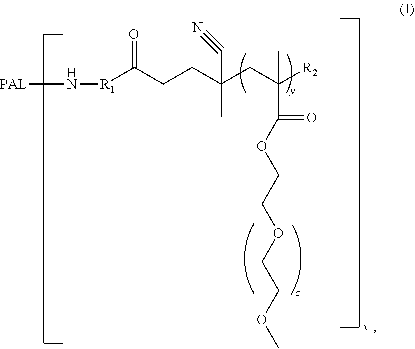

wherein:
PAL is a prokaryotic phenylalanine ammonia-lyase, which is a homotetrameric protein comprising four subunits;
R₁ is a linker;
R₂ is an end cap;
x is an integer from 1 to about 32;
y is an integer from about 50 to about 500; and
z is an integer from 2 to about 10.

In one embodiment, x is about 1 to about 28. In one embodiment, x is about 1 to about 24. In one embodiment, x is about 16 to about 32. In one embodiment, x is about 16 to about 28. In one embodiment, x is about 16 to about 24.

In one embodiment, the POEGMA-PAL conjugate is represented by Formula (I):

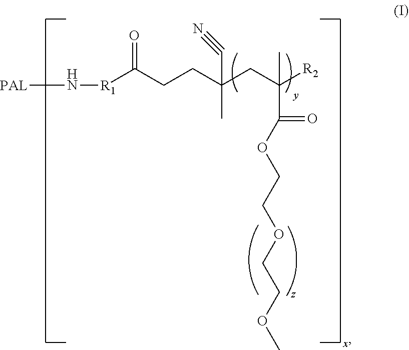

wherein:
PAL is a prokaryotic phenylalanine ammonia-lyase, which is a homotetrameric protein comprising four subunits;
R₁ is a linker;
R₂ is an end cap;
x is an integer from 1 to about 21;
y is an integer from about 50 to about 500; and
z is an integer from 2 to about 10.

As will be understood by one of ordinary skill in the art, Formula (I) can also be depicted as follows:

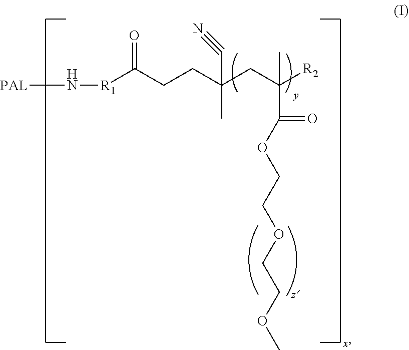

wherein z′ is an integer from 3 to about 11.

Formula (I) depicts POEGMA bound to PAL by way of NH—R₁. As will be recognized by one of skill in the art, “N” of the “NH” depicted in Formula (I) is the nitrogen of the side chain of a lysine residue of PAL to which the POEGMA-containing portion of Formula (I) is conjugated.

The “polymer containing portion of Formula (I),” includes R₁ and the POEGMA to which it is bound (i.e., R₁ and everything right of R₁) and will be referred herein to as “linker-POEGMA” as shown below in Formula (V):

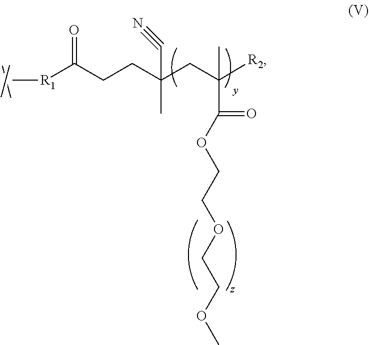

wherein:
[symbol] is the attachment to the nitrogen (N) directly bound to PAL in Formula (I);
R₁ is a linker;
R₂ is an end cap;
y is an integer from about 50 to about 500; and
z is an integer from 2 to about 10.

The portion of Formulas (I) and (V) to the right of and excluding linker R₁ will be referred to herein as the POEGMA, and is represented as shown below in Formula (VI):

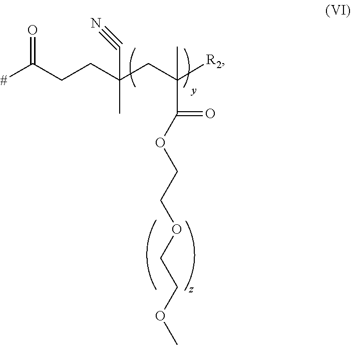

wherein:
R₁ is the attachment to R₁ in Formulas (I) and (V);
R₂ is an end cap;
y is an integer from about 50 to about 500; and
z is an integer from 2 to about 10.

In one embodiment of Formula (I), PAL is a wild-type PAL from a prokaryotic organism, including, but not limited to, PAL from Streptomyces maritimus, Nostoc punctiforme, Anabaena variabilis, Anacystis nidulans, Photorabdus luminescens TT01, and Streptomyces verticillatus. In one embodiment, PAL is a fragment, mutant, variant, or analog of a wild-type PAL enzyme that retains similar (e.g., at least 50%) or increased catalytic activity for phenylalanine. In one embodiment, PAL is a prokaryotic PAL from Anabaena variabilis (SEQ ID NO:1) or biologically active fragment, mutant, variant or analog thereof. In certain embodiments, PAL is an Anabaena variabilis PAL (AvPAL) variant. In one embodiment, PAL is an AvPAL variant wherein one or more amino acid residues have been substituted by another amino acid residue. In some embodiments, one or more cysteine residues of the AvPAL variant have been substituted by a serine residue. In some embodiments, the one or more cysteine residues of the AvPAL variant that have been substituted by one or more serine residues are selected from the group consisting of cysteine residues at positions 503 and 565. In one embodiment, each subunit of PAL has an amino acid sequence according to SEQ ID NO:2. In one embodiment, each subunit of PAL has an amino acid sequence according to SEQ ID NO:3. In one embodiment, each subunit of PAL has an amino acid sequence according to SEQ ID NO:4.

The chemical structure of linker R₁ in Formulas (I) and (V) is not particularly limited and may be any structure that covalently binds POEGMA to PAL. In one embodiment, R₁ is C₁₋₁₂ alkyl, C(O)—C₁₋₁₂ alkyl-NH, C(O)—(O—CH₂—CH₂) n, wherein n is 2 or 3, or a peptide cleavable spacer, such as Gly-Gly-Phe-Gly (SEQ ID NO:5). In one embodiment, R₁ is C(O)—C₁₋₁₂ alkyl-NH. In one embodiment, R₁ is C(O)—(CH₂)₅—NH.

In one embodiment, R₁ is CH₂, C(O)—C₁₋₁₂ alkyl-NH, C(O)—(O—CH₂—CH₂) n, wherein n is 2 or 3, or a peptide cleavable spacer, such as Gly-Gly-Phe-Gly (SEQ ID NO:5). In one embodiment, R₁ is C(O)—C₁₋₁₂ alkyl-NH. In one embodiment, R₁ is C(O)—(CH₂)₅—NH.

The identity of end cap R₂ in Formulas (I), (V), and (VI) is likewise not particularly limited and one of skill in the art will recognize any number of end caps that may be suitable for use in the conjugates disclosed herein. In one embodiment, R₂ is CN-substituted alkyl. In one embodiment, R₂ is isobutyronitrile, 4-cyanovaleric acid, 4-methoxy-2,4-dimethylvaleronitrile, or —OH (hydroxy). In one embodiment, R₂ is isobutyronitrile.

The variable x in Formula (I) refers to the number of linker-POEGMAs of Formula (V) that are conjugated to a single PAL enzyme.

In one embodiment, x is an integer from about 1 to about 32, about 2 to about 32, about 3 to about 32, about 4 to about 32, about 5 to about 32, about 6 to about 32, about 7 to about 32, about 8 to about 32, about 9 to about 32, about 10 to about 32, about 11 to about 32, about 12 to about 32, about 13 to about 32, about 14 to about 32, about 15 to about 32, about 16 to about 32, about 17 to about 32, about 18 to about 32, about 19 to about 32, about 20 to about 32, about 21 to about 32, about 22 to about 32, about 23 to about 32, about 24 to about 32, about 25 to about 32, about 26 to about 32, about 27 to about 32, about 28 to about 32, about 29 to about 32, or about 31 to about 32.

In one embodiment, x is an integer from about 1 to about 28, about 2 to about 28, about 3 to about 28, about 4 to about 28, about 5 to about 28, about 6 to about 28, about 7 to about 28, about 8 to about 28, about 9 to about 28, about 10 to about 28, about 11 to about 28, about 12 to about 28, about 13 to about 28, about 14 to about 28, about 15 to about 28, about 16 to about 28, about 17 to about 28, about 18 to about 28, about 19 to about 28, about 20 to about 28, about 21 to about 28, about 22 to about 28, about 23 to about 28, about 24 to about 28, about 25 to about 28, about 26 to about 28, or about 27 to about 28.

In one embodiment, x is an integer from about 1 to about 24, about 2 to about 24, about 3 to about 24, about 4 to about 24, about 5 to about 24, about 6 to about 24, about 7 to about 24, about 8 to about 24, about 9 to about 24, about 10 to about 24, about 11 to about 24, about 12 to about 24, about 13 to about 24, about 14 to about 24, about 15 to about 24, about 16 to about 24, about 17 to about 24, about 18 to about 24, about 19 to about 24, about 20 to about 24, about 21 to about 24, about 22 to about 24, or about 23 to about 24.

In one embodiment, x is an integer from about 5 to about 21, about 8 to about 21, about 10 to about 21, about 16 to about 21, or about 19 to about 21. In one embodiment, x is 19. In one embodiment, x is 20. In one embodiment x is 21.

The variable y in Formulas (I), (V), and (VI) refers to the number of poly(ethylene)methyl ether methacrylate monomers present in the POEGMA (i.e., the degree of polymerization). y generally depends on the degree of polymerization and can be adjusted by one skilled in the art. In one embodiment, y is an integer of about 50 to about 500. In one embodiment, y is about 150 to about 300. In one embodiment, y is about 175 to about 200.

In one embodiment, y is an integer of about 175 to about 250. In certain embodiments, y is about 200.

The variable z in Formulas (I), (V), and (VI) refers to the number of ethylene glycol moieties present in the side chain of the POEGMA. In one embodiment, z is an integer of 2 to about 10. In one embodiment, z is 2 to about 8. In one embodiment, z is 2 to about 5. In one embodiment, z is 2.

In one embodiment, z is an integer of 2 to about 9. In one embodiment, z is an integer of 2 to about 7. In one embodiment, z is an integer of 2 to about 6. In one embodiment, z is an integer of 2 to about 4. In one embodiment, z is 3. In certain embodiments, z is 2.

In one embodiment of Formulas (I), (V), and (VI), x is about 8 to 32, y is 150 to 300, and z is 2 to about 10, or x is about 8 to 32, y is 150 to 300, and z is 2 to about 8, or x is about 8 to 32, y is 150 to 300, and z is 2 to about 5, or x is about 8 to 32, y is 150 to 300, and z is 2.

In one embodiment of Formulas (I), (V), and (VI), x is about 8 to 28, y is 150 to 300, and z is 2 to about 10, or x is about 8 to 28, y is 150 to 300, and z is 2 to about 8, or x is about 8 to 28, y is 150 to 300, and z is 2 to about 5, or x is about 8 to 28, y is 150 to 300, and z is 2.

In one embodiment of Formulas (I), (V), and (VI), x is about 8 to 24, y is 150 to 300, and z is 2 to about 10, or x is about 8 to 24, y is 150 to 300, and z is 2 to about 8, or x is about 8 to 24, y is 150 to 300, and z is 2 to about 5, or x is about 8 to 24, y is 150 to 300, and z is 2.

In one embodiment of Formulas (I), (V), and (VI), x is about 8 to 21, y is 150 to 300, and z is 2 to about 10, or x is about 8 to 21, y is 150 to 300, and z is 2 to about 8, or x is about 8 to 21, y is 150 to 300, and z is 2 to about 5, or x is about 8 to 21, y is 150 to 300, and z is 2.

In one embodiment of Formulas (I), (V), and (VI), x is about 10 to 21, y is 150 to 300, and z is 2 to about 10, or x is about 10 to 21, y is 150 to 300, and z is 2 to about 8, or x is about 10 to 21, y is 150 to 300, and z is 2 to about 5, or x is about 10 to 21, y is 150 to 300, and z is 2.

In one embodiment of Formulas (I), (V), and (VI), x is about 16 to 21, y is 150 to 300, and z is 2 to about 10, or x is about 16 to 21, y is 150 to 300, and z is 2 to about 8, or x is about 16 to 21, y is 150 to 300, and z is 2 to about 5, or x is about 16 to 21, y is 150 to 300, and z is 2.

In one embodiment of Formulas (I), (V), and (VI), x is about 16 to 24, y is 150 to 300, and z is 2 to about 10, or x is about 16 to 21, y is 150 to 300, and z is 2 to about 8, or x is about 16 to 21, y is 150 to 300, and z is 2 to about 5, or x is about 16 to 21, y is 150 to 300, and z is 2.

In one embodiment of Formulas (I), (V), and (VI), x is about 16 to 28, y is 150 to 300, and z is 2 to about 10, or x is about 16 to 21, y is 150 to 300, and z is 2 to about 8, or x is about 16 to 21, y is 150 to 300, and z is 2 to about 5, or x is about 16 to 21, y is 150 to 300, and z is 2.

In one embodiment of Formulas (I), (V), and (VI), x is about 19 to 21, y is 150 to 300, and z is 2 to about 10, or x is about 19 to 21, y is 150 to 300, and z is 2 to about 8, or x is about 19 to 21, y is 150 to 300, and z is 2 to about 5, or x is about 19 to 21, y is 150 to 300, and z is 2.

In one embodiment of Formulas (I), (V), and (VI), x is about 8 to 21, y is 175 to 200, and z is 2 to about 10, or x is about 8 to 21, y is 175 to 200, and z is 2 to about 10, or x is about 8 to 21, y is 175 to 200, and z is 2 to about 10, or x is about 8 to 21, y is 175 to 200, and z is 2.

In one embodiment of Formulas (I), (V), and (VI), x is about 10 to 21, y is 175 to 200, and z is 2 to about 10, or x is about 10 to 21, y is 175 to 200, and z is 2 to about 10, or x is about 10 to 21, y is 175 to 200, and z is 2 to about 10, or x is about 19 to 21, y is 175 to 200, and z is 2.

In one embodiment of Formulas (I), (V), and (VI), x is about 16 to 21, y is 175 to 200, and z is 2 to about 10, or x is about 16 to 21, y is 175 to 200, and z is 2 to about 10, or x is about 16 to 21, y is 175 to 200, and z is 2 to about 10, or x is about 16 to 21, y is 175 to 200, and z is 2.

In one embodiment of Formulas (I), (V), and (VI), x is about 19 to 21, y is 175 to 200, and z is 2 to about 10, or x is about 19 to 21, y is 175 to 200, and z is 2 to about 10, or x is about 19 to 21, y is 175 to 200, and z is 2 to about 10, or x is about 19 to 21, y is 175 to 200, and z is 2.

In one embodiment of Formulas (I), (V), and (VI), x is 19, y is 50 to 500, and z is 2 to about 10; or x is 20, y is 50 to 500, and z is 2 to about 10; or x is 21, y is 50 to 500, and z is 2 to about 10. In one embodiment of Formulas (I), (V), and (VI), x is 19, y is 150 to 300, and z is 2 to about 10; or x is 20, y is 150 to 300, and z is 2 to about 10; or x is 21, y is 150 to 300, and z is 2 to about 10. In one embodiment of Formulas (I), (V), and (VI), x is 19, y is 175 to 200, and z is 2 to about 10; or x is 20, y is 175 to 200, and z is 2 to about 10; or x is 21, y is 175 to 200, and z is 2 to about 10.

In one embodiment of Formulas (I), (V), and (VI), x is 19, y is 50 to 500, and z is 2 to about 8; or x is 20, y is 50 to 500, and z is 2 to about 10; or x is 21, y is 50 to 500, and z is 2 to about 8. In one embodiment of Formulas (I), (V), and (VI), x is 19, y is 150 to 300, and z is 2 to about 8; or x is 20, y is 150 to 300, and z is 2 to about 10; or x is 21, y is 150 to 300, and z is 2 to about 8. In one embodiment of Formulas (I), (V), and (VI), x is 19, y is 175 to 200, and z is 2 to about 8; or x is 20, y is 175 to 200, and z is 2 to about 10; or x is 21, y is 175 to 200, and z is 2 to about 8.

In one embodiment of Formulas (I), (V), and (VI), x is 19, y is 50 to 500, and z is 2 to about 5; or x is 20, y is 50 to 500, and z is 2 to about 10; or x is 21, y is 50 to 500, and z is 2 to about 5. In one embodiment of Formulas (I), (V), and (VI), x is 19, y is 150 to 300, and z is 2 to about 5; or x is 20, y is 150 to 300, and z is 2 to about 10; or x is 21, y is 150 to 300, and z is 2 to about 5. In one embodiment of Formulas (I), (V), and (VI), x is 19, y is 175 to 200, and z is 2 to about 5; or x is 20, y is 175 to 200, and z is 2 to about 10; or x is 21, y is 175 to 200, and z is 2 to about 5.

In one embodiment of Formulas (I), (V), and (VI), x is 19, y is 50 to 500, and z is 2; or x is 20, y is 50 to 500, and z is 2; or x is 21, y is 50 to 500, and z is 2. In one embodiment of Formulas (I), (V), and (VI), x is 19, y is 150 to 300, and z is 2; or x is 20, y is 150 to 300, and z is 2; or x is 21, y is 150 to 300, and z is 2. In one embodiment of Formulas (I), (V), and (VI), x is 19, y is 175 to 200, and z is 2; or x is 20, y is 175 to 200, and z is 2; or x is 21, y is 175 to 200, and z is 2.

In one embodiment of Formulas (I), (V), and (VI), x is about 20, y is about 200 and z is 2.

In one embodiment, R₁ is C(O)—C₁₋₁₂ alkyl-NH. In one embodiment, the conjugate of Formula (I) is represented by Formula (I′):

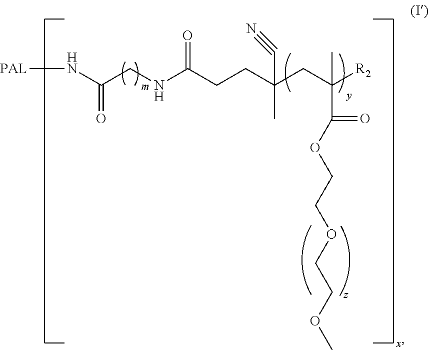

wherein:
PAL is a prokaryotic phenylalanine ammonia-lyase, which is a homotetrameric protein comprising four subunits;
R₂ is an end cap;
m is an integer from about 3 to about 12;
x is an integer from 1 to about 32;
y is an integer from about 50 to about 500; and
z is an integer from 2 to about 10.

In one embodiment, x is about 1 to about 28. In one embodiment, x is about 1 to about 24. In one embodiment, x is about 16 to about 32. In one embodiment, x is about 16 to about 28. In one embodiment, x is about 16 to about 24.

In one embodiment, R₁ is C(O)—C₁₋₁₂ alkyl-NH. In one embodiment, the conjugate of Formula (I) is represented by Formula (I′):

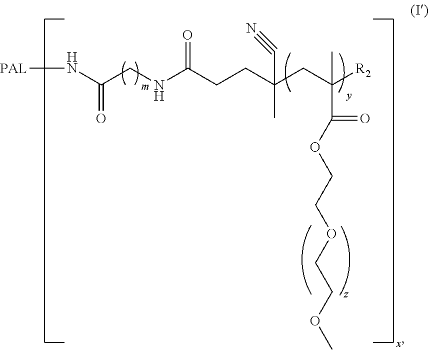

wherein:
PAL is a prokaryotic phenylalanine ammonia-lyase, which is a homotetrameric protein comprising four subunits;
R₂ is an end cap;
m is an integer from about 3 to about 12;
x is an integer from 1 to about 21;
y is an integer from about 50 to about 500; and
z is an integer from 2 to about 10.

In one embodiment, R₁ is C(O)—C₁₋₆ alkyl-NH. In one embodiment, the POEGMA-PAL conjugate of Formula (I) is represented by Formula (Ia):

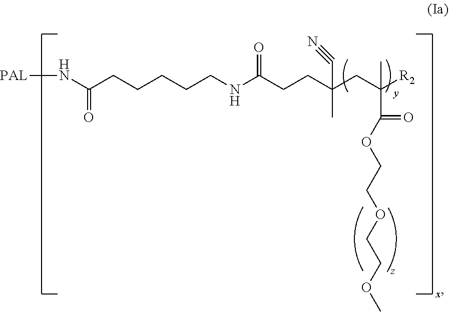

wherein:
PAL is a prokaryotic phenylalanine ammonia-lyase, which is a homotetrameric protein comprising four subunits;
R₂ is an end cap;
x is an integer from 1 to about 32;
y is an integer from about 50 to about 500; and
z is an integer from 2 to about 10.

In one embodiment, x is about 1 to about 28. In one embodiment, x is about 1 to about 24. In one embodiment, x is about 16 to about 32. In one embodiment, x is about 16 to about 28. In one embodiment, x is about 16 to about 24.

In one embodiment, R₁ is C(O)—C₁₋₆ alkyl-NH. In one embodiment, the POEGMA-PAL conjugate of Formula (I) is represented by Formula (Ia):

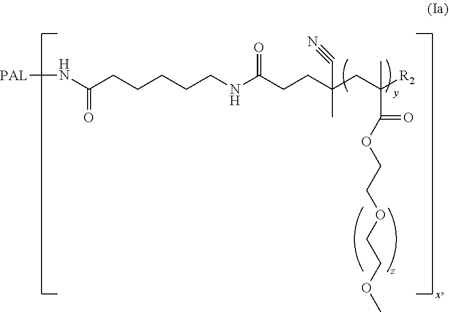

wherein:
PAL is a prokaryotic phenylalanine ammonia-lyase, which is a homotetrameric protein comprising four subunits;
R₂ is an end cap;
x is an integer from 1 to about 21;
y is an integer from about 50 to about 500; and
z is an integer from 2 to about 10.

In one embodiment, the POEGMA-PAL conjugate of Formula (I) is represented by Formula (Ia-1):

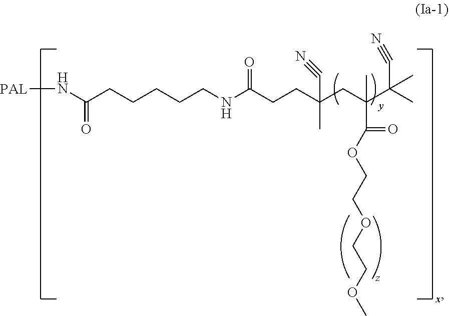

wherein:
PAL is a prokaryotic phenylalanine ammonia-lyase, which is a homotetrameric protein comprising four subunits;
x is an integer from 1 to about 32;
y is an integer from about 50 to about 500; and
z is an integer from 2 to about 10.

In one embodiment, x is about 1 to about 28. In one embodiment, x is about 1 to about 24. In one embodiment, x is about 16 to about 32. In one embodiment, x is about 16 to about 28. In one embodiment, x is about 16 to about 24.

In one embodiment, the POEGMA-PAL conjugate of Formula (I) is represented by Formula (Ia-1):

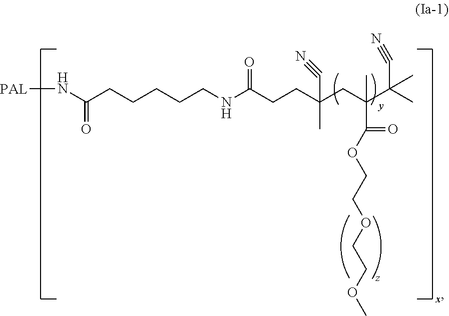

wherein:
PAL is a prokaryotic phenylalanine ammonia-lyase, which is a homotetrameric protein comprising four subunits;
x is an integer from 1 to about 21;
y is an integer from about 50 to about 500; and
z is an integer from 2 to about 10.

In one embodiment, the POEGMA-PAL conjugate of Formula (I) is represented by Formula (Ia-1-1):

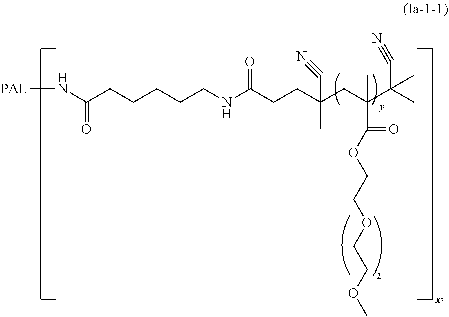

wherein:
PAL is a prokaryotic phenylalanine ammonia-lyase, which is a homotetrameric protein comprising four subunits;
x is an integer from 1 to about 32; and
y is an integer from about 50 to about 500.

In one embodiment, x is about 1 to about 28. In one embodiment, x is about 1 to about 24. In one embodiment, x is about 16 to about 32. In one embodiment, x is about 16 to about 28. In one embodiment, x is about 16 to about 24.

In one embodiment, the POEGMA-PAL conjugate of Formula (I) is represented by Formula (Ia-1-1):

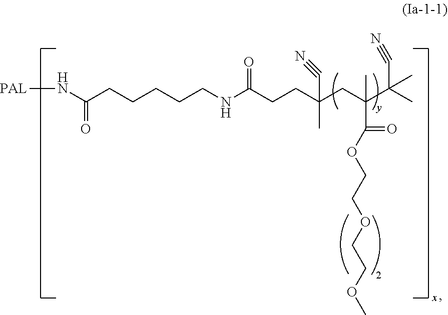

wherein:
PAL is a prokaryotic phenylalanine ammonia-lyase, which is a homotetrameric protein comprising four subunits;
x is an integer from 1 to about 21; and
y is an integer from about 50 to about 500.

In one embodiment of Formulas (I), (I′), (Ia), (Ia-1), (Ia-1-1), and/or any of (Ia-1-11)-(Ia-1-46) described in Table A, PAL is a wild-type PAL from a prokaryotic organism, including, but not limited to, PAL from Streptomyces maritimus, Nostoc punctiforme, Anabaena variabilis, Anacystis nidulans, Photorabdus luminescens TT01, and Streptomyces verticillatus. In one embodiment, PAL is a fragment, mutant, variant, or analog of a wild-type PAL enzyme that retains similar (e.g., at least 50%) or increased catalytic activity for phenylalanine. In one embodiment, PAL is a prokaryotic PAL from Anabaena variabilis (SEQ ID NO:1) or biologically active fragment, mutant, variant or analog thereof. In certain embodiments, PAL is an Anabaena variabilis PAL (AvPAL) variant. In one embodiment, PAL is an AvPAL variant wherein one or more amino acid residues have been substituted by another amino acid residue. In some embodiments, one or more cysteine residues of the AvPAL variant have been substituted by a serine residue. In some embodiments, the one or more cysteine residues of the AvPAL variant that have been substituted by one or more serine residues are selected from the group consisting of cysteine residues at positions 503 and 565. In one embodiment, each subunit of PAL has an amino acid sequence according to SEQ ID NO:2. In one embodiment, PAL has an amino acid sequence according to SEQ ID NO: 3. In one embodiment, each subunit of PAL has an amino acid sequence according to SEQ ID NO:4.

In one embodiment of Formulas (I′) and (Ia), R₂ is isobutyronitrile, 4-cyanovaleric acid, 4-methoxy-2,4-dimethylvaleronitrile, or hydroxy (—OH). In one embodiment, R₂ is isobutyronitrile.

x, y, and z may be as defined above for Formulas (I), (V), and (VI). In one embodiment of Formulas (I′), (Ia), (Ia-1), and (Ia-1-1), x is an integer from about 5 to about 32, about 8 to about 32, about 10 to about 32, about 16 to about 32, or about 19 to about 32, about 24 to about 32, or about 28 to about 32. In one embodiment of Formulas (I′), (Ia), (Ia-1), and (Ia-1-1), x is an integer from about 5 to about 28, about 8 to about 28, about 10 to about 28, about 16 to about 28, about 19 to about 28, or about 24 to about 28. In one embodiment of Formulas (I′), (Ia), (Ia-1), and (Ia-1-1), x is an integer from about 5 to about 24, about 8 to about 24, about 10 to about 24, about 16 to about 24, or about 19 to about 24. In one embodiment, y is an integer from about 50 to about 500, such as about 150 to about 300 or about 175 to about 200. In one embodiment, z is an integer from 2 to about 10, such as 2 to about 8 or 2 to about 5. In one embodiment, z is 2. Any combination of x, y, and z is encompassed by the present disclosure.

x, y, and z may be as defined above for Formulas (I), (V), and (VI). In one embodiment of Formulas (I′), (Ia), (Ia-1), and (Ia-1-1), x is an integer from about 5 to about 21, about 8 to about 21, about 10 to about 21, about 16 to about 21, or about 19 to about 21. In one embodiment, x is 19. In one embodiment, x is 20. In one embodiment x is 21. In one embodiment, y is an integer from about 50 to about 500, such as about 150 to about 300 or about 175 to about 200. In one embodiment, z is an integer from 2 to about 10, such as 2 to about 8 or 2 to about 5. In one embodiment, z is 2. Any combination of x, y, and z is encompassed by the present disclosure.

In one embodiment of Formulas (I′), (Ia), (Ia-1), and (Ia-1-1), 5-10 of the lysine residues of PAL are each conjugated to a linker-POEGMA. In one embodiment of Formulas (I′), (Ia), (Ia-1), and (Ia-1-1), at least 10 of the lysine residues of PAL are each conjugated to a linker-POEGMA. In one embodiment of Formulas (I′), (Ia), (Ia-1), and (Ia-1-1), 10-16 lysine residues of PAL are each conjugated to a linker-POEGMA. In one embodiment of Formulas (I′), (Ia), (Ia-1), and (Ia-1-1), at least 16 of the lysine residues of PAL are each conjugated to a linker-POEGMA. In one embodiment, 16-21 of the lysine residues of PAL are each conjugated to a linker-POEGMA. In one embodiment, at least 19 of the lysine residues of PAL are each conjugated to a linker-POEGMA. In one embodiment, 19-21 of the lysine residues of PAL are each conjugated to a linker-POEGMA. In one embodiment, at least 5 lysine residues on each subunit of PAL are each conjugated to a linker-POEGMA. In one embodiment, 5 or 6 lysine residues on each subunit of PAL are each conjugated to a linker-POEGMA.

In one embodiment, 16-32 of the lysine residues of PAL are each conjugated to a linker-POEGMA. In one embodiment, 16-28 of the lysine residues of PAL are each conjugated to a linker-POEGMA. In one embodiment, 16-24 of the lysine residues of PAL are each conjugated to a linker-POEGMA. In one embodiment, 18-22 of the lysine residues of PAL are each conjugated to a linker-POEGMA. In one embodiment, at least 20 of the lysine residues of PAL are each conjugated to a linker-POEGMA. In one embodiment, at least 21 of the lysine residues of PAL are each conjugated to a linker-POEGMA. In one embodiment, at least 22 of the lysine residues of PAL are each conjugated to a linker-POEGMA. In one embodiment, at least 23 of the lysine residues of PAL are each conjugated to a linker-POEGMA. In one embodiment, at least 24 of the lysine residues of PAL are each conjugated to a linker-POEGMA. In one embodiment, at least 6 lysine residues on each subunit of PAL are each conjugated to a linker-POEGMA. In one embodiment, 6 or 7 lysine residues on each subunit of PAL are each conjugated to a linker-POEGMA. In one embodiment, at least 7 lysine residues on each subunit of PAL are each conjugated to a linker-POEGMA. In one embodiment, 7 or 8 lysine residues on each subunit of PAL are each conjugated to a linker-POEGMA.

In some embodiments of Formulas (I), (I′), (Ia), (Ia-1), and (Ia-1-1), the lysine residues on each subunit of PAL that are conjugated to a linker-POEGMA comprise independently (e.g., may be different for each subunit) one or more (e.g., 1-9) of K189, K195, K522, K413, K145, K32, K115, K335, and K109, although a person of skill in the art will understand that in some embodiments, other lysine residues of PAL may also be conjugated to a linker-POEGMA. In some embodiments of Formulas (I), (I′), (Ia), (Ia-1), and (Ia-1-1), the lysine residues on each subunit of PAL that are conjugated to a linker-POEGMA comprise independently (e.g., may be different for each subunit) two or more (e.g., 2-9) of K189, K195, K522, K413, K145, K32, K115, K335, and K109, although a person of skill in the art will understand that in some embodiments, other lysine residues of PAL may also be conjugated to a linker-POEGMA. In some embodiments of Formulas (I), (I′), (Ia), (Ia-1), and (Ia-1-1), the lysine residues on each subunit of PAL that are conjugated to a linker-POEGMA comprise independently (e.g., may be different for each subunit) three or more (e.g., 3-9) of K189, K195, K522, K413, K145, K32, K115, K335, and K109, although a person of skill in the art will understand that in some embodiments, other lysine residues of PAL may also be conjugated to a linker-POEGMA. In some embodiments of Formulas (I), (I′), (Ia), (Ia-1), and (Ia-1-1), the lysine residues on each subunit of PAL that are conjugated to a linker-POEGMA comprise independently (e.g., may be different for each subunit) four or more (e.g., 4-9) of K189, K195, K522, K413, K145, K32, K115, K335, and K109, although a person of skill in the art will understand that in some embodiments, other lysine residues of PAL may also be conjugated to a linker-POEGMA.

In some embodiments of Formulas (I), (I′), (Ia), (Ia-1), and (Ia-1-1), the lysine residues on each subunit of PAL that are conjugated to a linker-POEGMA comprise independently (e.g., may be different for each subunit) five or more (e.g., 5-9) of K189, K195, K522, K413, K145, K32, K115, K335, and K109, although a person of skill in the art will understand that in some embodiments, other lysine residues of PAL may also be conjugated to a linker-POEGMA. In some embodiments of Formulas (I), (I′), (Ia), (Ia-1), and (Ia-1-1), the lysine residues on each subunit of PAL that are conjugated to a linker-POEGMA comprise independently (e.g., may be different for each subunit) six or more (e.g., 6-9) of K189, K195, K522, K413, K145, K32, K115, K335, and K109, although a person of skill in the art will understand that in some embodiments, other lysine residues of PAL may also be conjugated to a linker-POEGMA. In some embodiments of Formulas (I), (I′), (Ia), (Ia-1), and (Ia-1-1), the lysine residues on each subunit of PAL that are conjugated to a linker-POEGMA comprise independently (e.g., may be different for each subunit) seven or more (e.g., 7 or 8) of K189, K195, K522, K413, K145, K32, K115, K335, and K109, although a person of skill in the art will understand that in some embodiments, other lysine residues of PAL may also be conjugated to a linker-POEGMA.

In some embodiments, each of K189, K195 and K522 in at least one subunit of PAL (e.g., 1-4 subunits) are conjugated to a linker-POEGMA. In some embodiments, each of K189, K195 and K522 in at least two subunits of PAL (e.g., 2-4 subunits) are conjugated to a linker-POEGMA. In some embodiments, each of K189, K195 and K522 in at least three subunits of PAL (e.g., 3 or 4 subunits) are conjugated to a linker-POEGMA. In some embodiments, each of K189, K195 and K522 in each subunit of PAL are conjugated to a linker-POEGMA.

In some embodiments, each of K189, K195, K522, K413, and K145 in at least one subunit of PAL (e.g., 1-4 subunits) are conjugated to a linker-POEGMA. In some embodiments, each of K189, K195, K522, K413, and K145 in at least two subunits of PAL (e.g., 2-4 subunits) are conjugated to a linker-POEGMA. In some embodiments, each of K189, K195, K522, K413, and K145 in at least three subunits of PAL (e.g., 3 or 4 subunits) are conjugated to a linker-POEGMA. In some embodiments, each of K189, K195, K522, K413, and K145 in each subunit of PAL are conjugated to a linker-POEGMA.

In one embodiment, the POEGMA-PAL conjugate of Formula (I) is represented by any one of Formulas (Ia-1-11)-Formulas (Ia-1-46) as shown in Table A:

TABLE A
Exemplary POEGMA-PAL Conjugates

| | |
|---|---|
| Ia-1-11 | 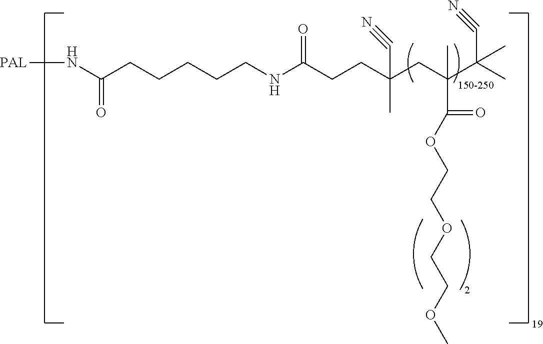 |
| Ia-1-12 | 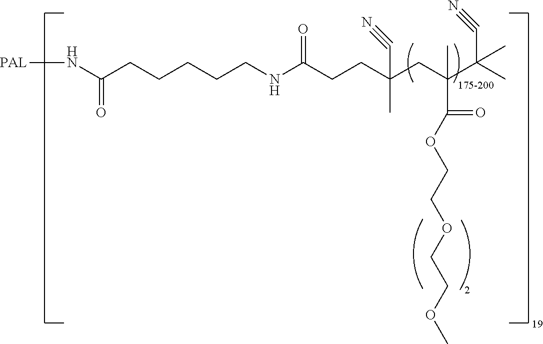 |
| Ia-1-13 | 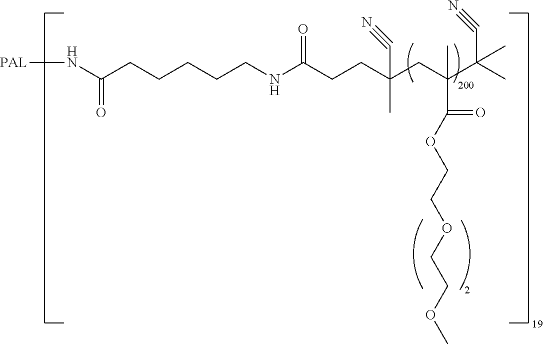 |
| Ia-1-14 | 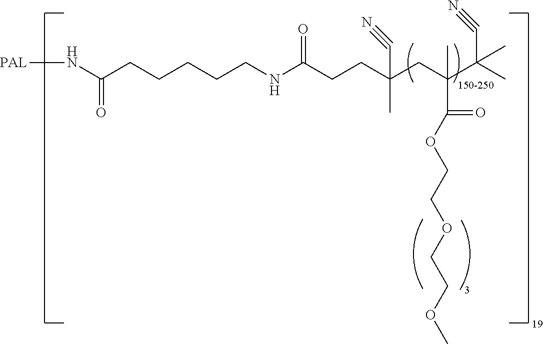 |
| Ia-1-15 | 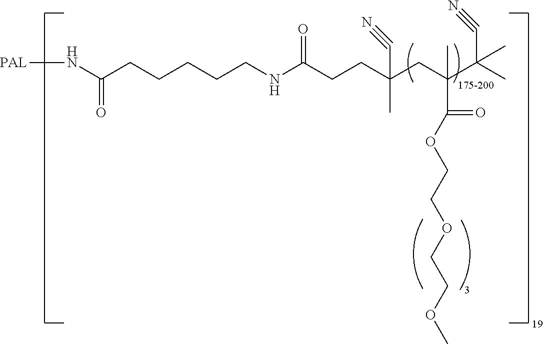 |
| Ia-1-16 | 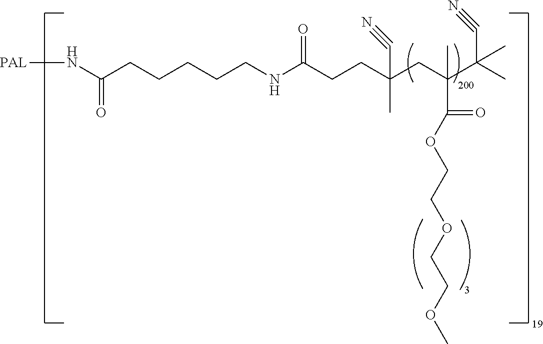 |
| Ia-1-17 | 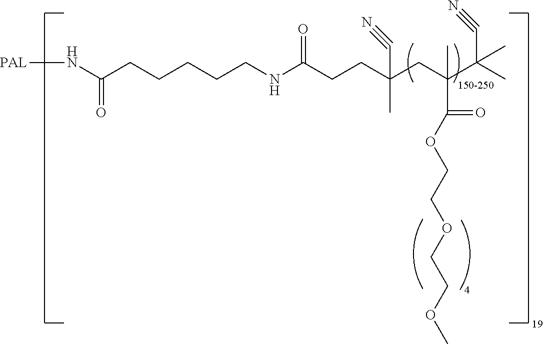 |
| Ia-1-18 | 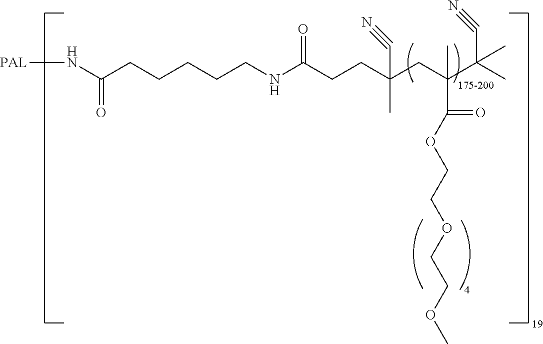 |
| Ia-1-19 | 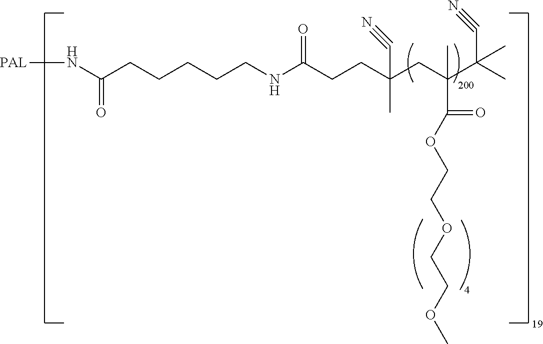 |
| Ia-1-20 | 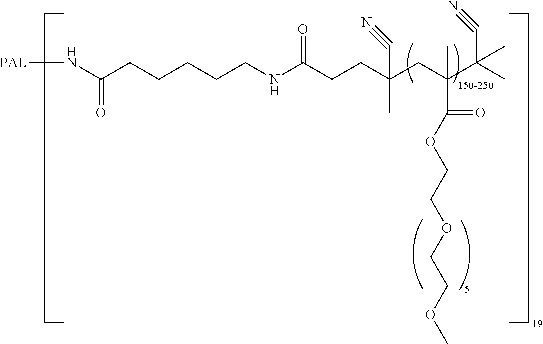 |
| Ia-1-21 | 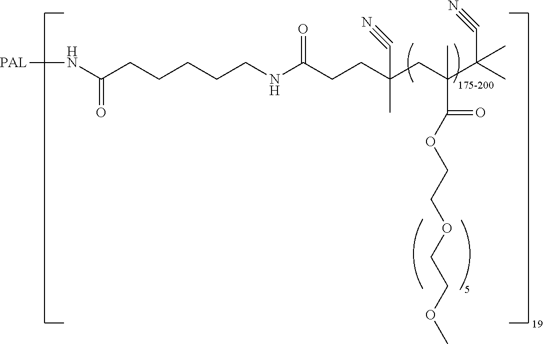 |
| Ia-1-22 | 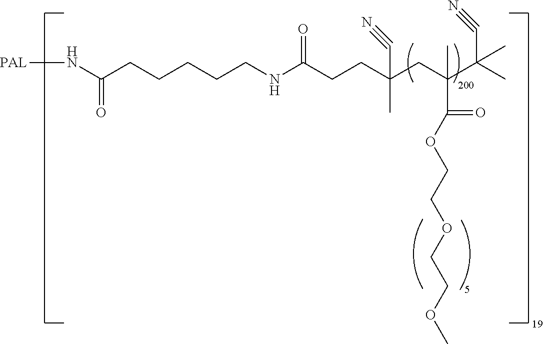 |
| Ia-1-23 | 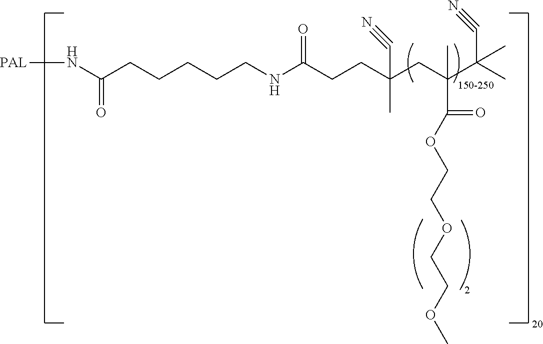 |
| Ia-1-24 | 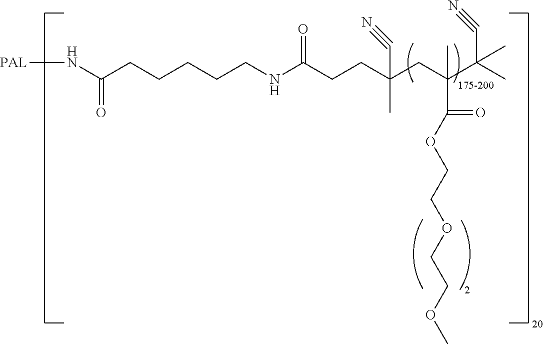 |
| Ia-1-25 | 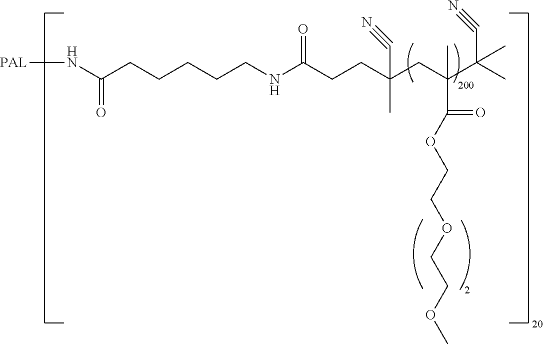 |
| Ia-1-26 | 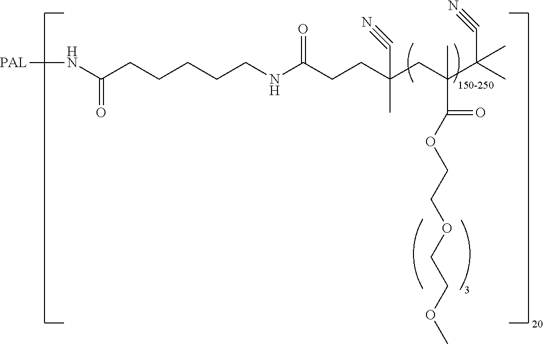 |
| Ia-1-27 | 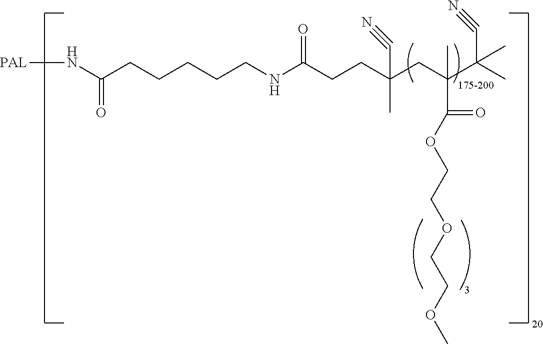 |
| Ia-1-28 | 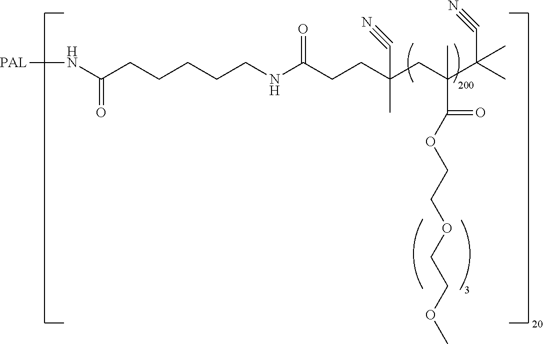 |
| Ia-1-29 | 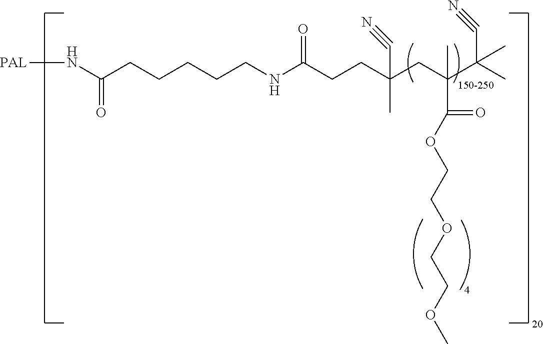 |
| Ia-1-30 | 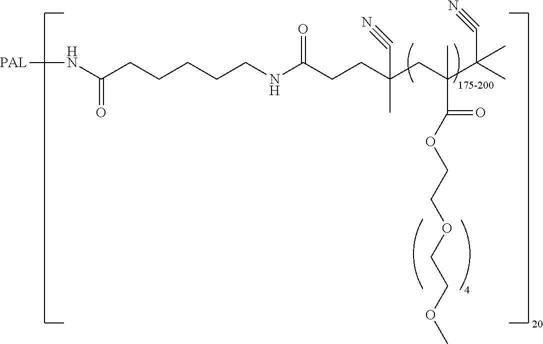 |
| Ia-1-31 | 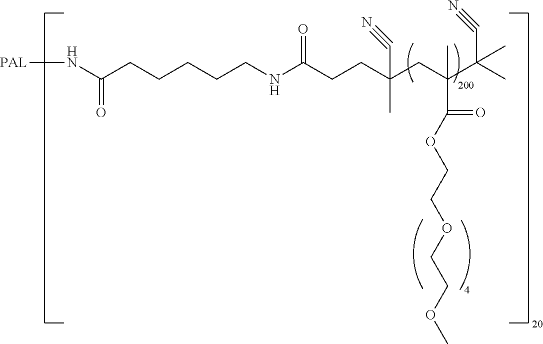 |
| Ia-1-32 | 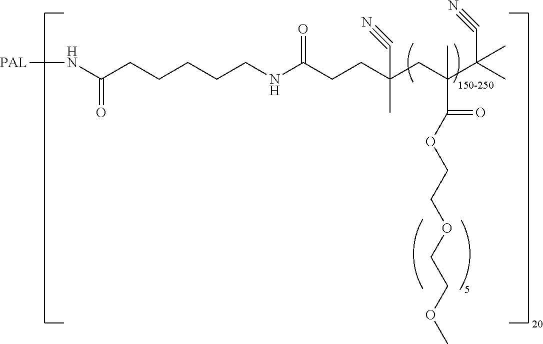 |
| Ia-1-33 | 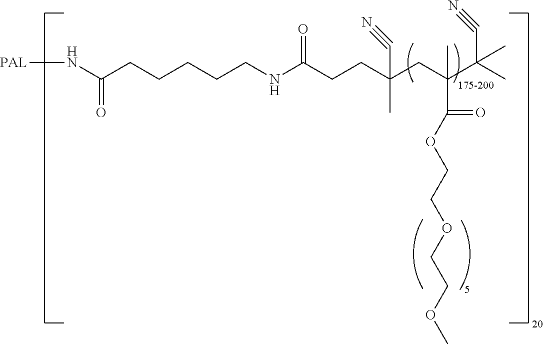 |
| Ia-1-34 | 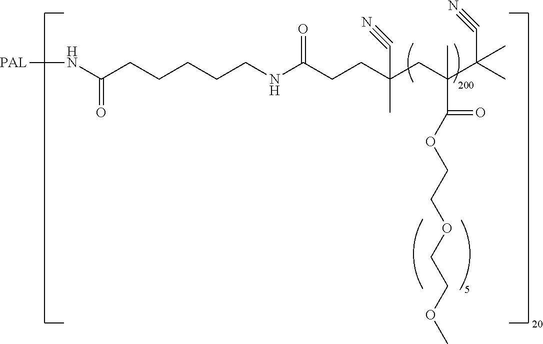 |
| Ia-1-35 | 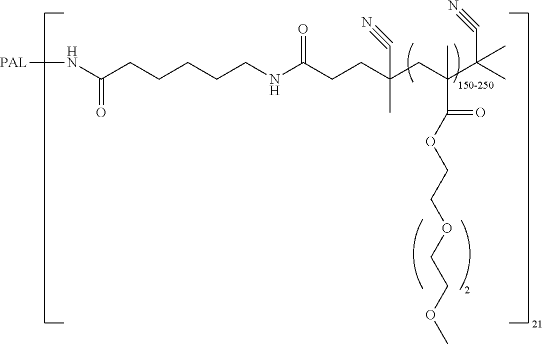 |
| Ia-1-36 | 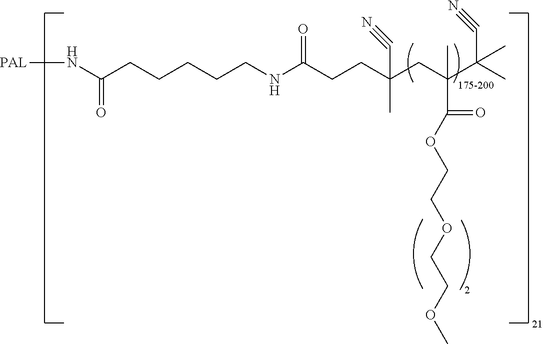 |
| Ia-1-37 | 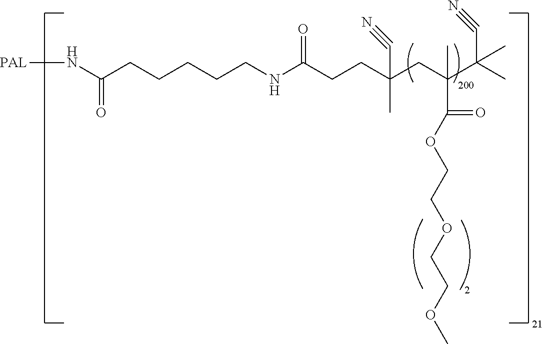 |
| Ia-1-38 | 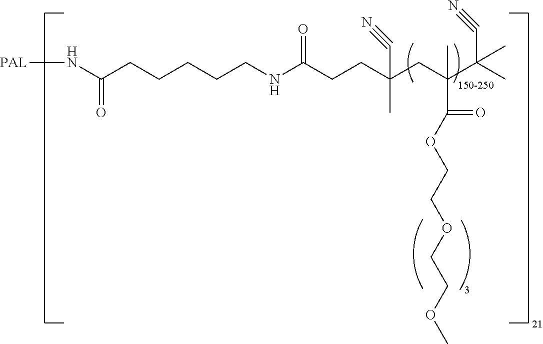 |
| Ia-1-39 | 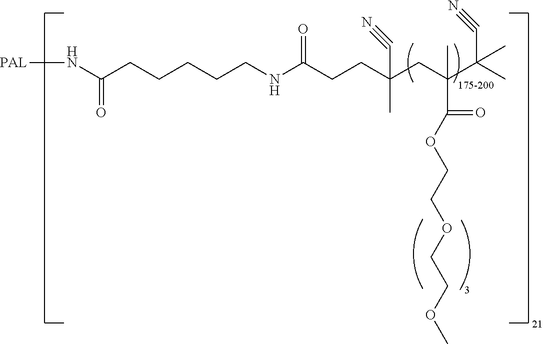 |
| Ia-1-40 | 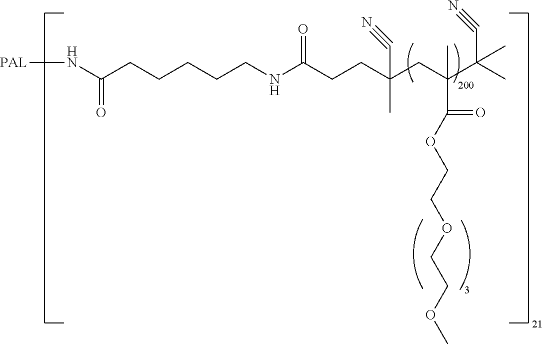 |
| Ia-1-41 | 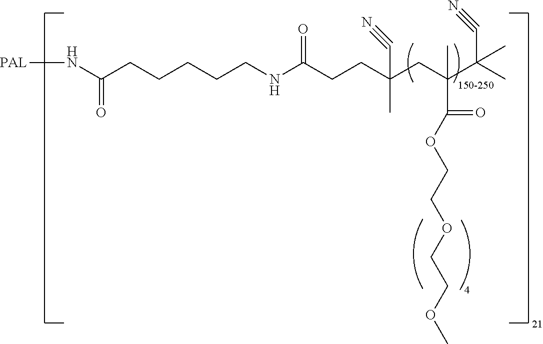 |
| Ia-1-42 | 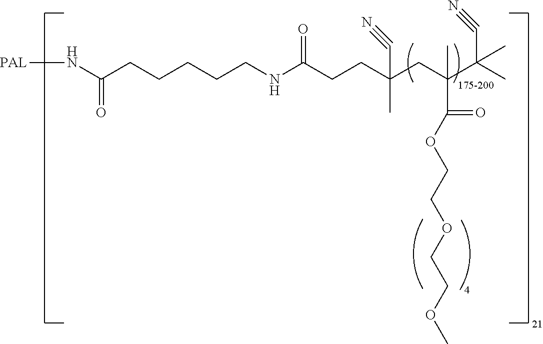 |
| Ia-1-43 | 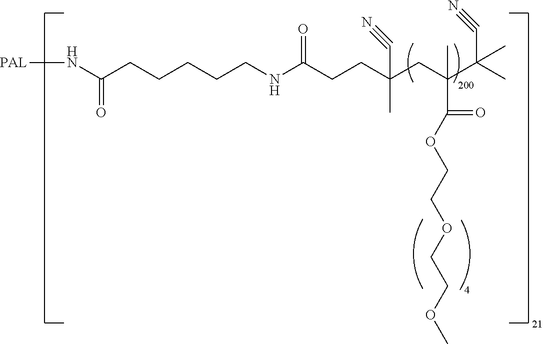 |
| Ia-1-44 | 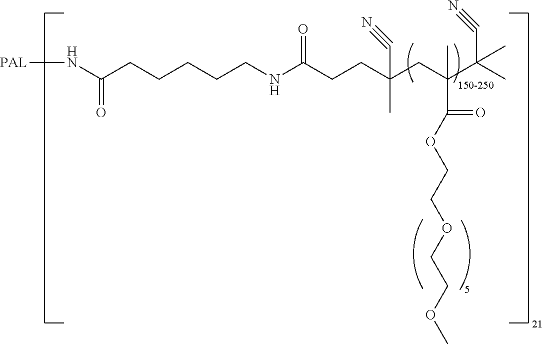 |
| Ia-1-45 | 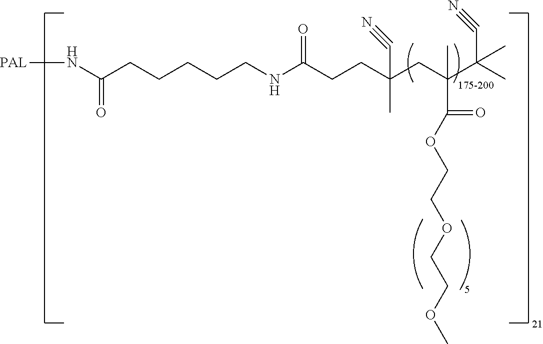 |
| Ia-1-46 | 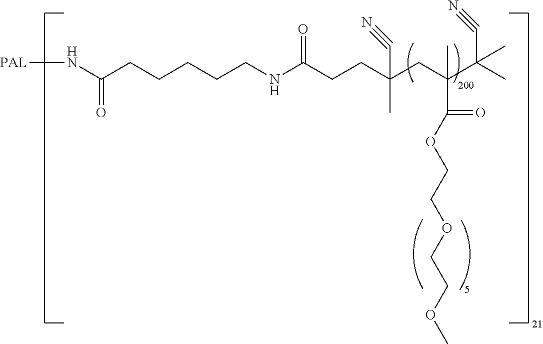 |

### 5.2 Methods of Making POEGMA-PAL Conjugates

A POEGMA-PAL conjugate, as described by any of the embodiments disclosed herein, may be prepared by preparing a POEGMA polymer and subsequently conjugating the POEGMA polymer to PAL. Therefore, in another aspect, the present disclosure provides a method of making a POEGMA-PAL conjugate of Formula (I), wherein the method comprises:
a. providing a pre-conjugation polymer of Formula (IV):

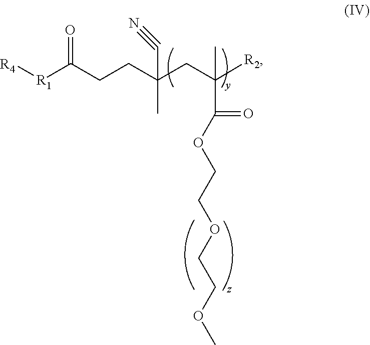

wherein:
   y is an integer from about 50 to about 500;
   z is an integer from 2 to about 10;
   R₁ is a linker;
   R₂ is an end cap; and
   R₄ is a conjugation moiety; and
b. contacting the pre-conjugation polymer of Formula (IV) with PAL under conditions effective to conjugate the pre-conjugation polymer of Formula (IV) to PAL, thereby generating a POEGMA-PAL conjugate of Formula (I):

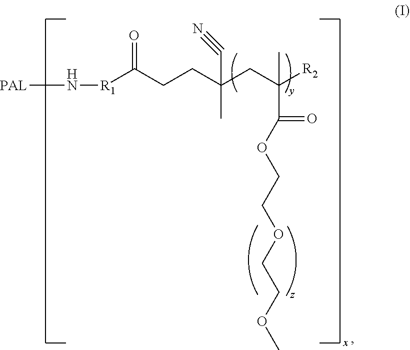

wherein PAL is a prokaryotic phenylalanine ammonia-lyase, which is a homotetrameric protein comprising four subunits.

In one embodiment, PAL is a wild-type PAL from a prokaryotic organism, including, but not limited to, PAL from Streptomyces maritimus, Nostoc punctiforme, Anabaena variabilis, Anacystis nidulans, Photorabdus luminescens TT01, and Streptomyces verticillatus. In one embodiment, PAL is a fragment, mutant, variant, or analog of a wild-type PAL enzyme that retains similar (e.g., at least 50%) or increased catalytic activity for phenylalanine. In one embodiment, PAL is a prokaryotic PAL from Anabaena variabilis (SEQ ID NO:1) or biologically active fragment, mutant, variant or analog thereof. In certain embodiments, PAL is an Anabaena variabilis PAL (AvPAL) variant. In one embodiment, PAL is an AvPAL variant wherein one or more amino acid residues have been substituted by another amino acid residue. In some embodiments, one or more cysteine residues of the AvPAL variant have been substituted by a serine residue. In some embodiments, the one or more cysteine residues of the AvPAL variant that have been substituted by one or more serine residues are selected from the group consisting of cysteine residues at positions 503 and 565. In one embodiment, each subunit of PAL has an amino acid sequence according to SEQ ID NO:2. In one embodiment, PAL has an amino acid sequence according to SEQ ID NO: 3. In one embodiment, each subunit of PAL has an amino acid sequence according to SEQ ID NO:4.

The pre-conjugation polymer of Formula (IV) may be prepared by any method known in the art, such as chain-growth polymerization. In one embodiment, a pre-conjugation polymer of Formula (IV) is prepared using radical addition-fragmentation transfer (RAFT) polymerization.

In one embodiment, a POEGMA-PAL conjugate of Formula (I) may be prepared by a method comprising:
a. contacting polyethylene glycol methacrylate with a compound of Formula (II) under conditions effective to generate a polymer of Formula (III), as shown in SCHEME I:

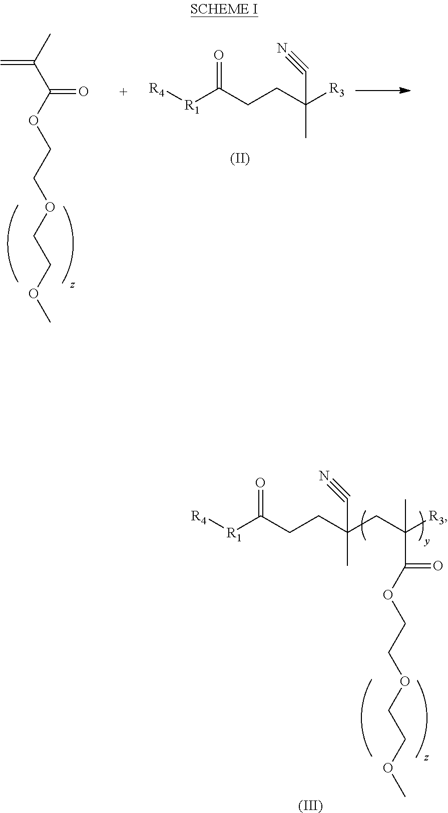

wherein:
   y is an integer from about 50 to about 500;
   z is an integer from 2 to about 10;
   R₁ is a linker;
   R₃ is a RAFT chain transfer agent; and
   R₄ is a conjugation moiety;
b. contacting the polymer of Formula (III) with an end group modification agent to generate a pre-conjugation polymer of Formula (IV) as shown in SCHEME II:

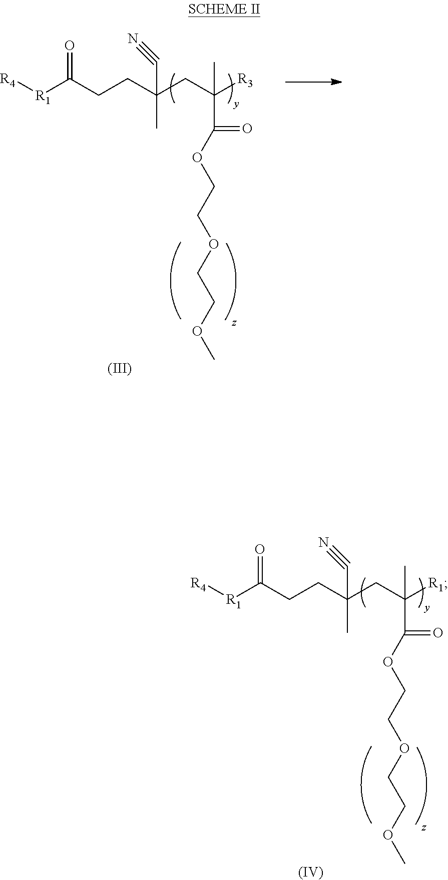

wherein R₂ is an end cap; and
c. contacting the pre-conjugation polymer of Formula (IV) with PAL under conjugation conditions effective to create a bond between R₁ and one or more lysine residue side chains (e.g., 1-21 lysine residue side chains) of the PAL, as shown in SCHEME III:

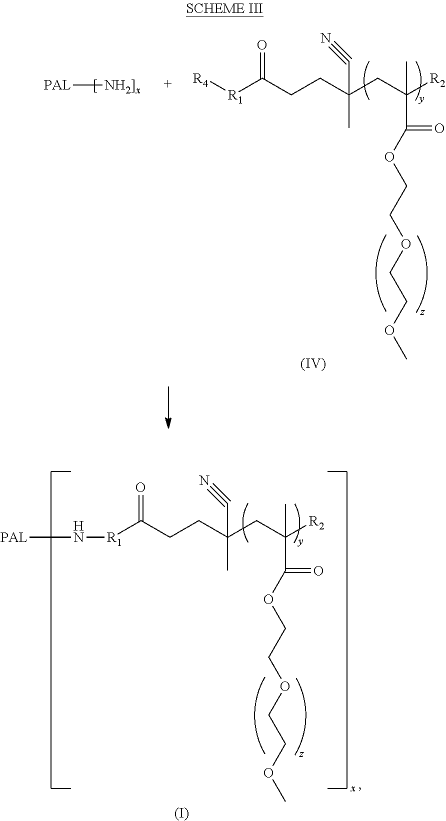

wherein PAL is the prokaryotic phenylalanine ammonia-lyase and x is an integer from 1 to about 21.

In SCHEMES I, II, and III, linker R₁ may be CH₂, C(O)—C₁₋₁₂ alkyl-NH, C(O)—(O—CH₂—CH₂)ₙ, wherein n is 2 or 3, or Gly-Gly-Phe-Gly (SEQ ID NO:5).

In one embodiment, R₁ is C(O)—(CH₂)₅—NH. In one embodiment, the above methods may be used to generate a POEGMA-PAL conjugate of Formula (Ia):

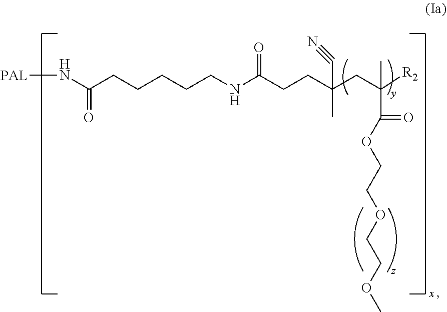

wherein:
PAL is a prokaryotic phenylalanine ammonia-lyase;
R₂ is an end cap;
x is an integer from 1 to about 21;
y is an integer from about 50 to about 500; and
z is an integer from 2 to about 10.

In one embodiment, end cap R₂ is isobutyronitrile, 4-cyanovaleric acid, 4-methoxy-2,4-dimethylvaleronitrile, or a hydroxy group (—OH). In one embodiment, R₂ is isobutyronitrile. In one embodiment, the above methods may be used to generate a POEGMA-PAL conjugate of Formula (Ia-1):

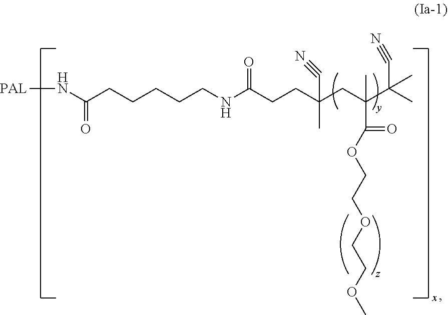

wherein:
PAL is a prokaryotic phenylalanine ammonia-lyase;
R₂ is an end cap;
x is an integer from 1 to about 21;
y is an integer from about 50 to about 500; and
z is an integer from 2 to about 10.

R₃ in Formulas (II) and (III) of SCHEMES I and II may be any RAFT chain transfer agent. One of ordinary skill in the art will be familiar with a variety of RAFT chain transfer agents that can be used, as well as the conditions effective to polymerize using such agents.

In one embodiment, the RAFT chain transfer agent is a dithiocarbamate, a trithiocarbonate, a xanthate, or a dithioester. In one embodiment, the RAFT chain transfer agent R₃ is a trithiocarbonate. In one embodiment, the RAFT chain transfer agent R₃ is

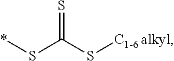

wherein the asterisk (*) represents attachment to the rest of the compound or polymer. In one embodiment, the RAFT chain transfer agent R₃ is

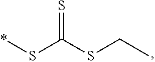 [SMILES: *SC(=S)SCC]

wherein the asterisk (*) represents attachment to the rest of the compound or polymer. In one embodiment, the conditions effective to generate a polymer of Formula (III) may comprise contacting the oligoethylene glycol methacrylate with the compound of Formula (II) in the presence of DMSO and a light source. In one embodiment, the light source is blue LED light.

By contacting the polymer of Formula (III) with an end group modification agent as shown in SCHEME II, R₃ may be removed from the polymer of Formula (III) to generate a pre-conjugation polymer of Formula (IV) with end cap R₂. The identity of the end group modification agent is not particularly limited and one of ordinary skill in the art will recognize suitable methods and reagents for capping the polymer suitably.

In one embodiment, the polymer of Formula (III) may be contacted with 4′-azobis(4-cyanovaleric acid) to remove R₃ and incorporate 4-cyanovaleric acid as end cap R₂. In one embodiment, the polymer of Formula (III) may be contacted with 2,2′-azobis(4-methoxy-2,4-dimethylvaleronitrile) to remove R₃ and incorporate 4-methoxy-2,4-dimethylvaleronitrile as end cap R₂. In one embodiment the polymer of Formula (III) may be contacted with UV light to remove R₃ and incorporate a hydroxy group (—OH) as end cap R₂. In one embodiment, the polymer of Formula (III) is contacted with azobisisobutyronitrile (AIBN), optionally in the presences of DMSO, to generate a pre-conjugation polymer of Formula (IV-1) having an isobutyronitrile end cap, as shown below in SCHEME II-1:

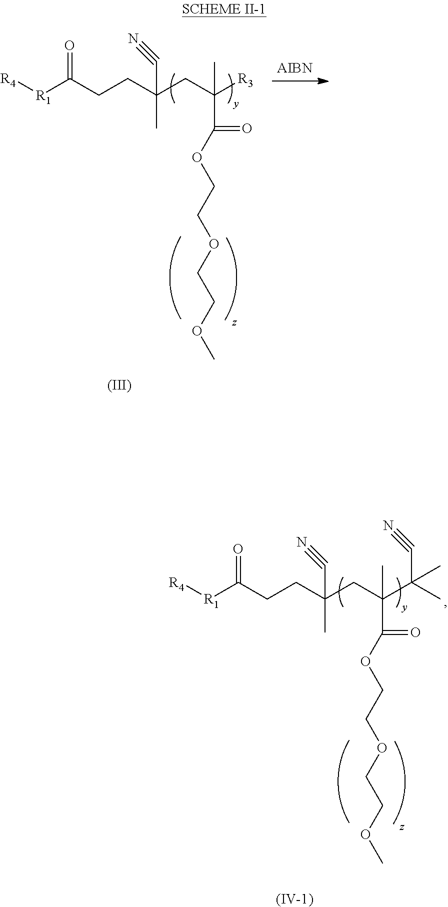

wherein:
y is an integer from about 50 to about 500;
z is an integer from 2 to about 10;
R₁ is a linker;
R₃ is a RAFT chain transfer agent; and
R₄ is a conjugation moiety.

In any of the Formulas depicted in SCHEMES I, II, and III, x, y and z may be as defined above for Formulas (I), (V), and (VI). In some embodiments, x may be an integer of 1 to about 21, about 8 to about 21, or about 16 to about 21; y may be an integer of about 50 to about 500, about 150 to about 300, or about 175 to about 200; and z may be an integer of 2 to about 10, 2 to about 8, or 2 to about 5. In one embodiment, zis 2. Any combination of x, y, and z are encompassed by the present disclosure.

Conjugation moiety R₄ in the compound of Formula (II), polymer of Formula (III), and pre-conjugation polymer of Formula (IV) is not particularly limited and may be any reactive moiety that can react with a lysine side chain of PAL to generate a bond between linker R₁ and the lysine side chain. Examples of non-limiting conjugation chemistries that may be used to conjugate the linker-POEGMA to PAL include tetra-fluorophenyl esters (TFP), penta-fluorophenyl esters (PFP), para-nitrophenol carbonates (p-NPC), and N-hydroxysuccinimide esters (NHS). For example, R₄ may be selected from the group consisting of

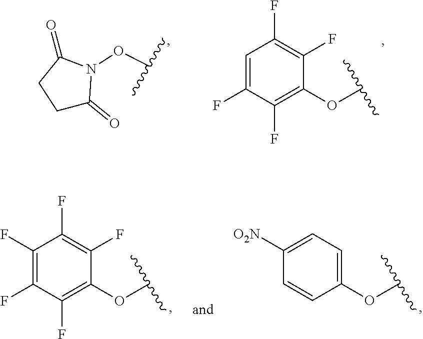

wherein [symbol] represents attachment to R₁.

One of skill in the art will be familiar with conditions effective for conjugating a pre-conjugation polymer of Formula (IV) having a particular conjugation moiety R₄, to PAL and will be able to perform such a reaction without undue experimentation. Further described below are exemplary, but not limiting, examples of various conjugation techniques that may be used according to the methods disclosed herein. Additionally, specific suitable reaction conditions are also discussed in the EXAMPLES.

In one embodiment, R₄ is

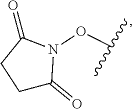

wherein [symbol] represents attachment to R₁ and a POEGMA-PAL conjugate of Formula (I) may be prepared by a method comprising contacting polyethylene glycol methacrylate with a compound of Formula (IIa) to prepare a polymer of Formula (IIIa), as shown in SCHEME Ia below:

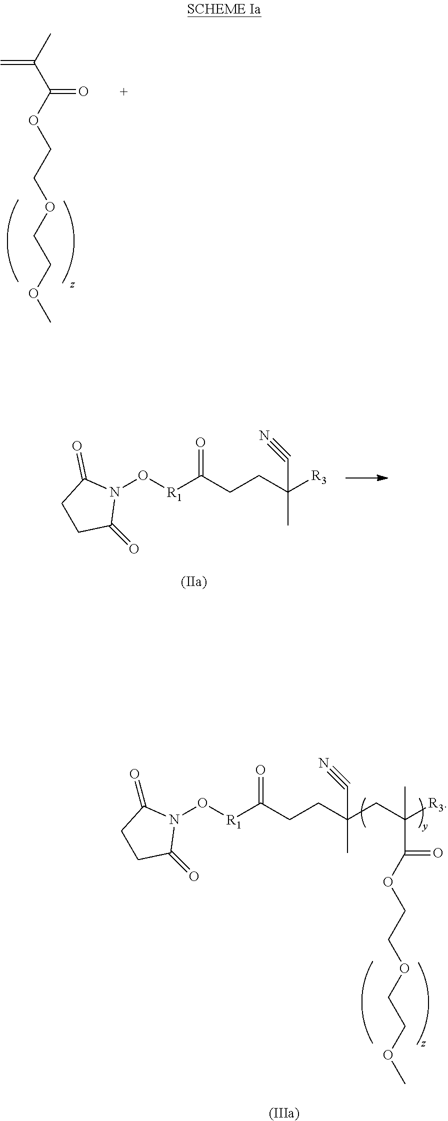

The polymer of Formula (IIIa) may then be contacted with an end group modification agent to remove R₃ to generate a pre-conjugation polymer of Formula (IVa) having end cap R₂, as shown in SCHEME IIa below:

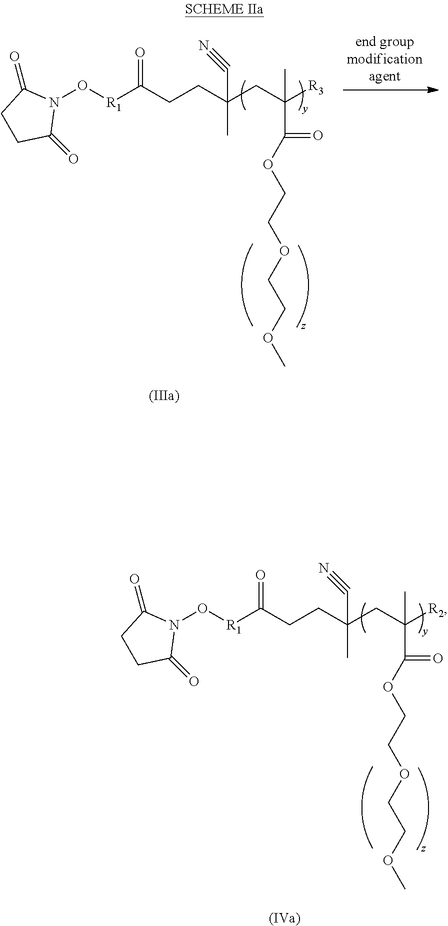

The pre-conjugation polymer of Formula (IVa) may then be contacted with PAL under conditions effective to conjugate at least one pre-conjugation polymer of Formula (IVa) to at least one lysine side chain of PAL, thereby generating a POEGMA-PAL conjugate of Formula (I), as shown in SCHEME IIIa below:

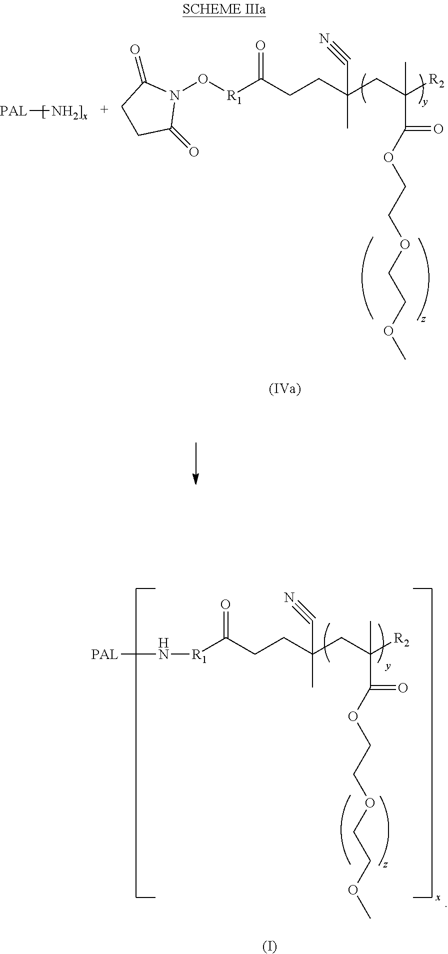

In each of SCHEMES Ia, IIa, and IIIa above:
PAL is a prokaryotic phenylalanine ammonia-lyase;
R₁ is a linker;
x is an integer from 1 to about 21;
y is an integer from about 50 to about 500; and
z is an integer from 2 to about 10.

In one embodiment, PAL is a wild-type PAL from a prokaryotic organism, including, but not limited to, PAL from Streptomyces maritimus, Nostoc punctiforme, Anabaena variabilis, Anacystis nidulans, Photorabdus luminescens TT01, and Streptomyces verticillatus. In one embodiment, PAL is a fragment, mutant, variant, or analog of a wild-type PAL enzyme that retains similar (e.g., at least 50%) or increased catalytic activity for phenylalanine. In one embodiment, PAL is a prokaryotic PAL from Anabaena variabilis (SEQ ID NO:1) or biologically active fragment, mutant, variant or analog thereof. In certain embodiments, PAL is an Anabaena variabilis PAL (AvPAL) variant. In one embodiment, PAL is an AvPAL variant wherein one or more amino acid residues have been substituted by another amino acid residue. In some embodiments, one or more cysteine residues of the AvPAL variant have been substituted by a serine residue. In some embodiments, the one or more cysteine residues of the AvPAL variant that have been substituted by one or more serine residues are selected from the group consisting of cysteine residues at positions 503 and 565. In one embodiment, each subunit of PAL has an amino acid sequence according to SEQ ID NO:2. In one embodiment, PAL has an amino acid sequence according to SEQ ID NO: 3. In one embodiment, each subunit of PAL has an amino acid sequence according to SEQ ID NO:4.

In certain embodiments, pre-conjugation polymer of Formula (IVa) is contacted with PAL under conditions effective to conjugate two or more pre-conjugation polymers of Formula (IVa) individually to two or more, e.g., 2-21, lysine side chains of PAL. In one embodiment, pre-conjugation polymer of Formula (IVa) is contacted with PAL under conditions effective to conjugate a pre-conjugation polymer of Formula (IVa) each individually to about 5 to about 21, about 8 to about 21, about 10 to about 21, about 16 to about 21, or about 19 to about 21 lysine side chains of PAL. In certain embodiments, pre-conjugation polymer of Formula (IVa) is contacted with PAL under conditions effective to conjugate a pre-conjugation polymer of Formula (IVa) each individually to about 19 to about 21 lysine side chains of PAL.

In certain embodiments, pre-conjugation polymer of Formula (IVa) is contacted with PAL under conditions effective to conjugate one or more, e.g., 1-21, pre-conjugation polymers of Formula (IVa) individually to one or more lysine side chains of each subunit of PAL. In certain embodiments, pre-conjugation polymer of Formula (IVa) is contacted with PAL under conditions effective to conjugate two or more, e.g., 2-21, pre-conjugation polymers of Formula (IVa) individually to two or more lysine side chains of each subunit of PAL. In certain embodiments, pre-conjugation polymer of Formula (IVa) is contacted with PAL under conditions effective to conjugate three or more, e.g., 3-21, pre-conjugation polymers of Formula (IVa) individually to three or more lysine side chains of each subunit of PAL. In certain embodiments, pre-conjugation polymer of Formula (IVa) is contacted with PAL under conditions effective to conjugate four or more, e.g., 4-21, pre-conjugation polymers of Formula (IVa) individually to four or more lysine side chains of each subunit of PAL.

In one embodiment, pre-conjugation polymer of Formula (IVa) is contacted with PAL under conditions effective to conjugate one or more, e.g., 1-5 or 1-6, linker-POEGMAs to one or more lysines on each subunit of PAL, wherein the one or more lysines on each subunit of PAL comprise independently (e.g., may be different for each subunit) one or more, e.g., 1-9, of K189, K195, K522, K413, K145, K32, K115, K335, and K109, although in some embodiments, one of ordinary skill in the art will understand that other lysine residues of PAL may also be conjugated to a linker-POEGMA. In one embodiment, pre-conjugation polymer of Formula (IVa) is contacted with PAL under conditions effective to conjugate two or more, e.g., 2-5 or 2-6, linker-POEGMAs each individually to two or more lysines on each subunit of PAL, wherein the two or more lysines on each subunit of PAL comprise independently (e.g., may be different for each subunit) two or more, e.g., 2-9, of K189, K195, K522, K413, K145, K32, K115, K335, and K109, although in some embodiments, one of ordinary skill in the art will understand that other lysine residues of PAL may also be conjugated to a linker-POEGMA. In one embodiment, a pre-conjugation polymer of Formula (IVa) is contacted with PAL under conditions effective to conjugate three or more, e.g., 3-5 or 3-6, linker-POEGMAs each individually to three or more lysines on each subunit of PAL, wherein the three or more lysines on each subunit of PAL comprise independently (e.g., may be different for each subunit) three or more, e.g., 3-9, of K189, K195, K522, K413, K145, K32, K115, K335, and K109, although in some embodiments, one of ordinary skill in the art will understand that other lysine residues of PAL may also be conjugated to a linker-POEGMA. In one embodiment, pre-conjugation polymer of Formula (IVa) is contacted with PAL under conditions effective to conjugate four or more, e.g., 4-5 or 4-6, linker-POEGMAs to four or more lysines on each subunit of PAL, wherein the four or more lysines on each subunit of PAL comprise independently (e.g., may be different for each subunit) four or more, e.g., 4-9, of K189, K195, K522, K413, K145, K32, K115, K335, and K109, although in some embodiments, one of ordinary skill in the art will understand that other lysine residues of PAL may also be conjugated to a linker-POEGMA. In one embodiment, pre-conjugation polymer of Formula (IVa) is contacted with PAL under conditions effective to conjugate five or more, e.g., 5 or 6, linker-POEGMAs each individually to five or more lysines on each subunit of PAL, wherein the five or more lysines on each subunit of PAL comprise independently (e.g., may be different for each subunit) four or more, e.g., 5-9, of K189, K195, K522, K413, K145, K32, K115, K335, and K109, although in some embodiments, one of ordinary skill in the art will understand that other lysine residues of PAL may also be conjugated to a linker-POEGMA.

In some embodiments, the conditions are effective to conjugate each of K189, K195 and K522 to a linker-POEGMA in at least one, e.g., 1-4, subunit of PAL. In some embodiments, the conditions are effective to conjugate each of K189, K195 and K522 to a linker-POEGMA in at least two subunits, e.g., 2-4, of PAL. In some embodiments, the conditions are effective to conjugate each of K189, K195 and K522 to a linker-POEGMA in at least three subunits, e.g., 3 or 4, of PAL. In some embodiments, the conditions are effective to conjugate each of K189, K195 and K522 to a linker-POEGMA in each subunit of PAL.

In some embodiments, pre-conjugation polymer of Formula (IVa) is contacted with PAL under conditions are effective to conjugate each of K189, K195, K522, K413, and K145 to a linker-POEGMA in at least one, e.g., 1-4, subunit of PAL. In some embodiments, the conditions are effective to conjugate each of K189, K195, K522, K413, and K145 to a linker-POEGMA in at least two, e.g., 2-4, subunits of PAL. In some embodiments, the conditions are effective to conjugate each of K189, K195, K522, K413, and K145 to a linker-POEGMA in at least three, e.g., 3 or 4, subunits of PAL. In some embodiments, the conditions are effective to conjugate each of K189, K195, K522, K413, and K145 to a linker-POEGMA in each subunit of PAL.

Conditions effective for conjugating the pre-conjugation polymer of Formula (IVa) containing an NHS conjugation moiety are well known to one of ordinary skill in the art. For example, a POEGMA-PAL conjugate of Formula (I) may be prepared by contacting PAL with a pre-conjugation polymer of Formula (IVa) in a solvent (e.g., an aqueous buffer) at a pH of about 7 to about 9.

In one embodiment, R₄ is

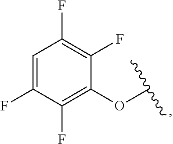

wherein [symbol] represents attachment to R₁ and a POEGMA-PAL conjugate of Formula (I) may be prepared by a method comprising contacting polyethylene glycol methacrylate with a compound of Formula (IIb) to prepare a polymer of Formula (IIIb), as shown in SCHEME Ib below:

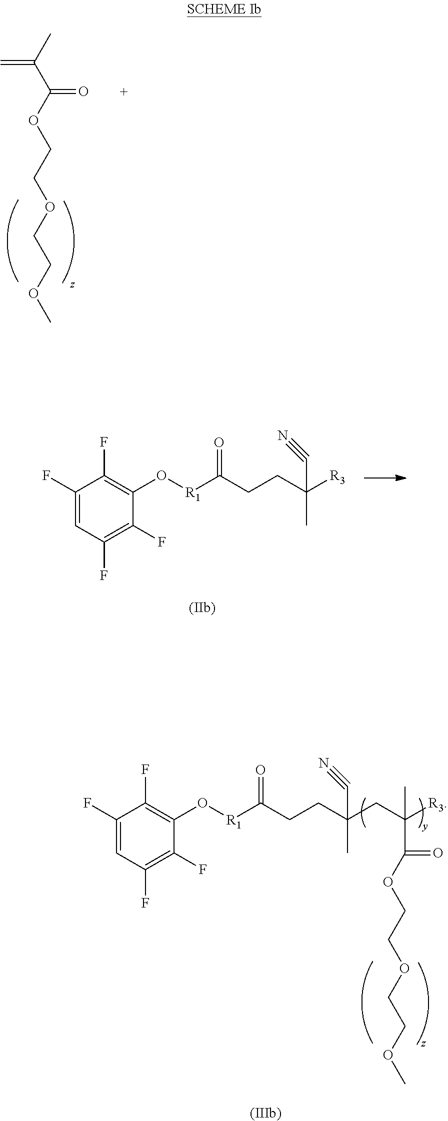

The polymer of formula (IIIb) may then be contacted with an end group modification agent to remove R₃ to generate a pre-conjugation polymer of Formula (IVb) having end cap R₂, as shown in SCHEME IIb below:

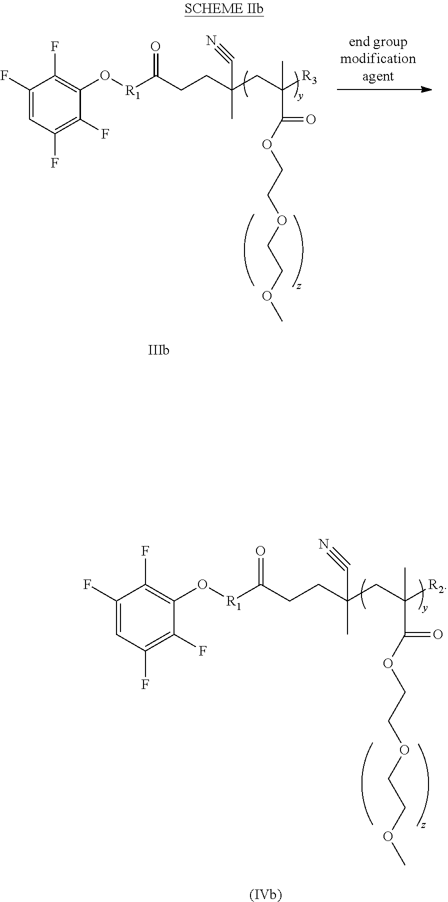

The pre-conjugation polymer of Formula (IVb) may then be contacted with PAL under conditions effective to conjugate the polymer of Formula (IVb) to PAL, thereby generating a POEGMA-PAL conjugate of Formula (I), as shown in SCHEME IIIb below:

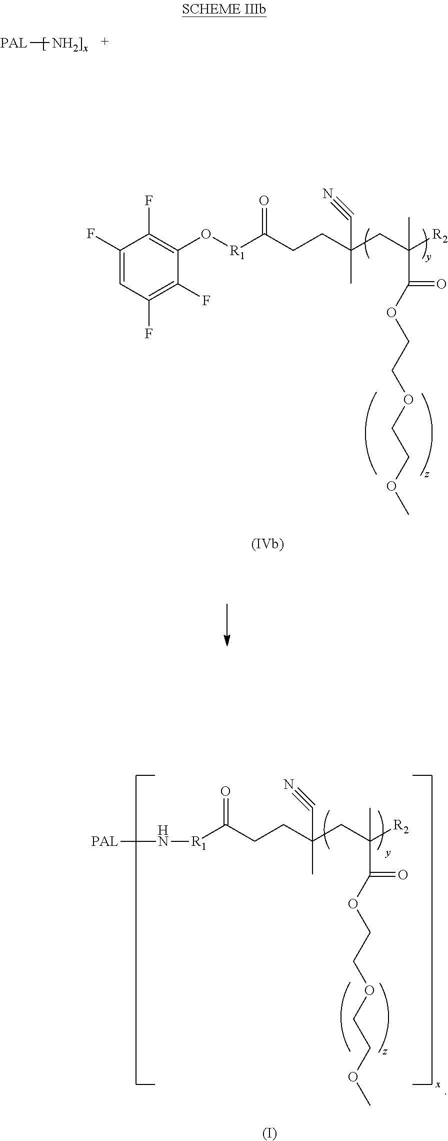

In each of SCHEMES Ib, IIb, and IIIb:
PAL is a prokaryotic phenylalanine ammonia-lyase;
R₁ is a linker;
x is an integer from 1 to about 21;
y is an integer from about 50 to about 500; and
z is an integer from 2 to about 10.

In one embodiment, PAL is a wild-type PAL from a prokaryotic organism, including, but not limited to, PAL from Streptomyces maritimus, Nostoc punctiforme, Anabaena variabilis, Anacystis nidulans, Photorabdus luminescens TT01, and Streptomyces verticillatus. In one embodiment, PAL is a fragment, mutant, variant, or analog of a wild-type PAL enzyme that retains similar (e.g., at least 50%) or increased catalytic activity for phenylalanine. In one embodiment, PAL is a prokaryotic PAL from Anabaena variabilis (SEQ ID NO:1) or biologically active fragment, mutant, variant or analog thereof. In certain embodiments, PAL is an Anabaena variabilis PAL (AvPAL) variant. In one embodiment, PAL is an AvPAL variant wherein one or more amino acid residues have been substituted by another amino acid residue. In some embodiments, one or more cysteine residues of the AvPAL variant have been substituted by a serine residue. In some embodiments, the one or more cysteine residues of the AvPAL variant that have been substituted by one or more serine residues are selected from the group consisting of cysteine residues at positions 503 and 565. In one embodiment, each subunit of PAL has an amino acid sequence according to SEQ ID NO:2. In one embodiment, PAL has an amino acid sequence according to SEQ ID NO: 3. In one embodiment, each subunit of PAL has an amino acid sequence according to SEQ ID NO:4.

In certain embodiments, pre-conjugation polymer of Formula (IVb) is contacted with PAL under conditions effective to conjugate two or more pre-conjugation polymers of Formula (IVb) individually to two or more, e.g., 2-21, lysine side chains of PAL. In one embodiment, pre-conjugation polymer of Formula (IVb) is contacted with PAL under conditions effective to conjugate a pre-conjugation polymer of Formula (IVb) each individually to about 5 to about 21, about 8 to about 21, about 10 to about 21, about 16 to about 21, or about 19 to about 21 lysine side chains of PAL. In one embodiment, pre-conjugation polymer of Formula (IVb) is contacted with PAL under conditions effective to conjugate a pre-conjugation polymer of Formula (IVb) each individually to about 19 to about 21 lysine side chains of PAL.

In certain embodiments, pre-conjugation polymer of Formula (IVb) is contacted with PAL under conditions effective to conjugate one or more, e.g., 1-21, pre-conjugation polymers of Formula (IVb) individually to one or more lysine side chains of each subunit of PAL. In certain embodiments, pre-conjugation polymer of Formula (IVb) is contacted with PAL under conditions effective to conjugate two or more, e.g., 2-21, pre-conjugation polymers of Formula (IVb) individually to two or more lysine side chains of each subunit of PAL. In certain embodiments, pre-conjugation polymer of Formula (IVb) is contacted with PAL under conditions effective to conjugate three or more, e.g., 3-21, pre-conjugation polymers of Formula (IVb) individually to three or more lysine side chains of each subunit of PAL. In certain embodiments, pre-conjugation polymer of Formula (IVb) is contacted with PAL under conditions effective to conjugate four or more, e.g., 4-21, pre-conjugation polymers of Formula (IVb) individually to four or more lysine side chains of each subunit of PAL.

In one embodiment, pre-conjugation polymer of Formula (IVb) is contacted with PAL under conditions effective to conjugate one or more, e.g., 1-5 or 1-6, linker-POEGMAs to one or more lysines on each subunit of PAL, wherein the one or more lysines on each subunit of PAL comprise independently (e.g., may be different for each subunit) one or more, e.g., 1-9, of K189, K195, K522, K413, K145, K32, K115, K335, and K109, although in some embodiments, one of ordinary skill in the art will understand that other lysine residues of PAL may also be conjugated to a linker-POEGMA. In one embodiment, pre-conjugation polymer of Formula (IVb) is contacted with PAL under conditions effective to conjugate two or more, e.g., 2-5 or 2-6, linker-POEGMAs to two one or more lysines on each subunit of PAL, wherein the two or more lysines on each subunit of PAL comprise independently (e.g., may be different for each subunit) two or more, e.g., 2-9, of K189, K195, K522, K413, K145, K32, K115, K335, and K109, although in some embodiments, one of ordinary skill in the art will understand that other lysine residues of PAL may also be conjugated to a linker-POEGMA. In one embodiment, a pre-conjugation polymer of Formula (IVb) is contacted with PAL under conditions effective to conjugate three or more, e.g., 3-5 or 3-6, linker-POEGMAs each individually to three or more lysines on each subunit of PAL, wherein the three or more lysines on each subunit of PAL comprise independently (e.g., may be different for each subunit) three or more, e.g., 3-9, of K189, K195, K522, K413, K145, K32, K115, K335, and K109, although in some embodiments, one of ordinary skill in the art will understand that other lysine residues of PAL may also be conjugated to a linker-POEGMA. In one embodiment, pre-conjugation polymer of Formula (IVb) is contacted with PAL under conditions effective to conjugate four or more, e.g., 4-5 or 4-6, linker-POEGMAs each individually to four or more lysines on each subunit of PAL, wherein the four or more lysines on each subunit of PAL comprise independently (e.g., may be different for each subunit) four or more, e.g., 4-9, of K189, K195, K522, K413, K145, K32, K115, K335, and K109, although in some embodiments, one of ordinary skill in the art will understand that other lysine residues of PAL may also be conjugated to a linker-POEGMA. In one embodiment, pre-conjugation polymer of Formula (IVb) is contacted with PAL under conditions effective to conjugate five or more, e.g., 5 or 6, linker-POEGMAs each individually to five or more lysines on each subunit of PAL, wherein the five or more lysines on each subunit of PAL comprise independently (e.g., may be different for each subunit) four or more, e.g., 5-9, of K189, K195, K522, K413, K145, K32, K115, K335, and K109, although in some embodiments, one of ordinary skill in the art will understand that other lysine residues of PAL may also be conjugated to a linker-POEGMA.

In some embodiments, the conditions are effective to conjugate each of K189, K195 and K522 to a linker-POEGMA in at least one, e.g., 1-4, subunit of PAL. In some embodiments, the conditions are effective to conjugate each of K189, K195 and K522 to a linker-POEGMA in at least two subunits, e.g., 2-4, of PAL. In some embodiments, the conditions are effective to conjugate each of K189, K195 and K522 to a linker-POEGMA in at least three subunits, e.g., 3 or 4, of PAL. In some embodiments, the conditions are effective to conjugate each of K189, K195 and K522 to a linker-POEGMA in each subunit of PAL.

In some embodiments, pre-conjugation polymer of Formula (IVb) is contacted with PAL under conditions are effective to conjugate each of K189, K195, K522, K413, and K145 to a linker-POEGMA in at least one, e.g., 1-4, subunit of PAL. In some embodiments, the conditions are effective to conjugate each of K189, K195, K522, K413, and K145 to a linker-POEGMA in at least two, e.g., 2-4, subunits of PAL. In some embodiments, the conditions are effective to conjugate each of K189, K195, K522, K413, and K145 to a linker-POEGMA in at least three, e.g., 3 or 4, subunits of PAL. In some embodiments, the conditions are effective to conjugate each of K189, K195, K522, K413, and K145 to a linker-POEGMA in each subunit of PAL.

Conditions effective for conjugating the pre-conjugation polymer of Formula (IVb) containing a tetrafluorophenyl ester conjugation moiety are well known to one of ordinary skill in the art. For example, a POEGMA-PAL conjugate of Formula (I) may be prepared by contacting PAL with a pre-conjugation polymer of Formula (IVb) in a solvent (e.g., an aqueous buffer) at a pH of about 7 to about 9.

In one embodiment, R₄ is

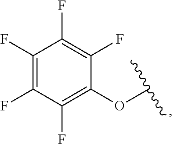

wherein [symbol] represents attachment to R₁ and a POEGMA-PAL conjugate of Formula (I) may be prepared by a method comprising contacting polyethylene glycol methacrylate with a compound of Formula (IIc) to prepare a polymer of Formula (IIIc), as shown in SCHEME Ic below:

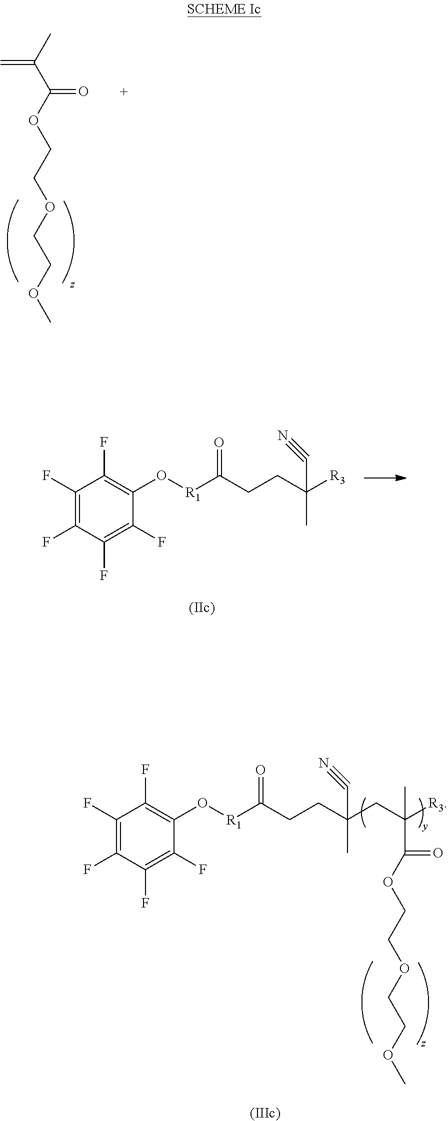

The polymer of formula (IIIc) may then be contacted with an end group modification agent to remove R₃ to generate a pre-conjugation polymer of Formula (IVc) having end cap R₂, as shown in SCHEME IIc below:

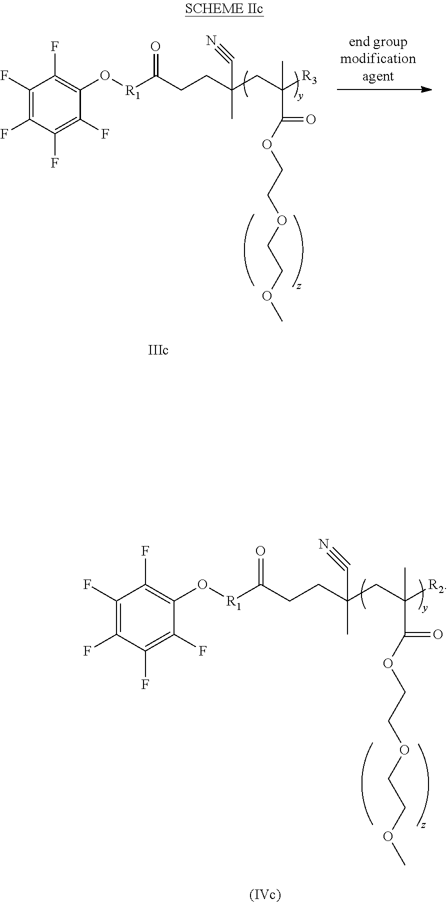

The pre-conjugation polymer of Formula (IVc) may then be contacted with PAL under conditions effective to conjugate the polymer of Formula (IVc) to PAL, thereby generating a POEGMA-PAL conjugate of Formula (I), as shown in as shown in SCHEME IIIc below:

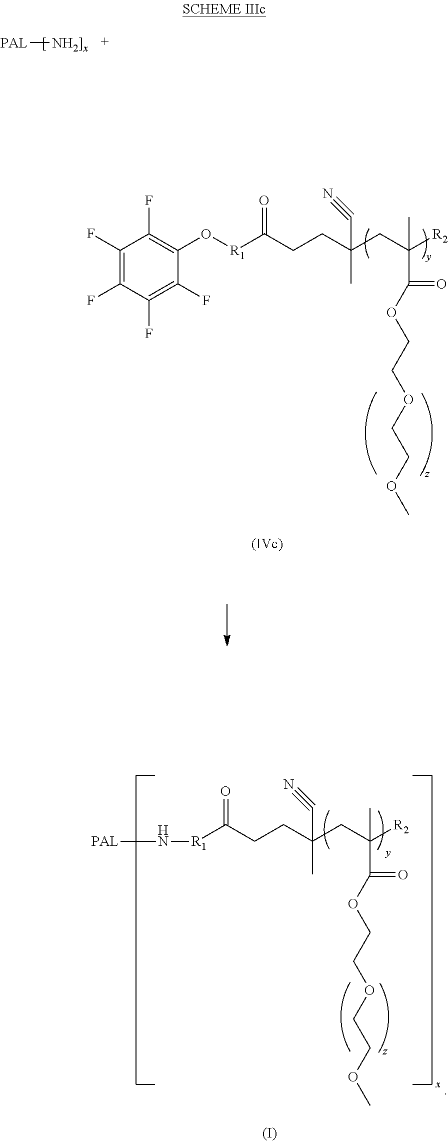

In each of SCHEMES Ic, IIc, and IIIc:
PAL is a prokaryotic phenylalanine ammonia-lyase;
R₁ is a linker;
x is an integer from 1 to about 21;
y is an integer from about 50 to about 500; and
z is an integer from 2 to about 10.

In one embodiment, PAL is a wild-type PAL from a prokaryotic organism, including, but not limited to, PAL from Streptomyces maritimus, Nostoc punctiforme, Anabaena variabilis, Anacystis nidulans, Photorabdus luminescens TT01, and Streptomyces verticillatus. In one embodiment, PAL is a fragment, mutant, variant, or analog of a wild-type PAL enzyme that retains similar (e.g., at least 50%) or increased catalytic activity for phenylalanine. In one embodiment, PAL is a prokaryotic PAL from Anabaena variabilis (SEQ ID NO:1) or biologically active fragment, mutant, variant or analog thereof. In certain embodiments, PAL is an Anabaena variabilis PAL (AvPAL) variant. In one embodiment, PAL is an AvPAL variant wherein one or more amino acid residues have been substituted by another amino acid residue. In some embodiments, one or more cysteine residues of the AvPAL variant have been substituted by a serine residue. In some embodiments, the one or more cysteine residues of the AvPAL variant that have been substituted by one or more serine residues are selected from the group consisting of cysteine residues at positions 503 and 565. In one embodiment, each subunit of PAL has an amino acid sequence according to SEQ ID NO:2. In one embodiment, PAL has an amino acid sequence according to SEQ ID NO: 3. In one embodiment, each subunit of PAL has an amino acid sequence according to SEQ ID NO:4.

In certain embodiments, pre-conjugation polymer of Formula (IVc) is contacted with PAL under conditions effective to conjugate two or more pre-conjugation polymers of Formula (IVc) individually to two or more, e.g., 2-21, lysine side chains of PAL. In one embodiment, pre-conjugation polymer of Formula (IVc) is contacted with PAL under conditions effective to conjugate a pre-conjugation polymer of Formula (IVc) each individually to about 5 to about 21, about 8 to about 21, about 10 to about 21, about 16 to about 21, or about 19 to about 21 lysine side chains of PAL. In one embodiment, pre-conjugation polymer of Formula (IVc) is contacted with PAL under conditions effective to conjugate a pre-conjugation polymer of Formula (IVc) each individually to about 19 to about 21 lysine side chains of PAL.

In certain embodiments, pre-conjugation polymer of Formula (IVc) is contacted with PAL under conditions effective to conjugate one or more, e.g., 1-21, pre-conjugation polymers of Formula (IVc) individually to one or more lysine side chains of each subunit of PAL. In certain embodiments, pre-conjugation polymer of Formula (IVc) is contacted with PAL under conditions effective to conjugate two or more, e.g., 2-21, pre-conjugation polymers of Formula (IVc) individually to two or more lysine side chains of each subunit of PAL. In certain embodiments, pre-conjugation polymer of Formula (IVc) is contacted with PAL under conditions effective to conjugate three or more, e.g., 3-21, pre-conjugation polymers of Formula (IVc) individually to three or more lysine side chains of each subunit of PAL. In certain embodiments, pre-conjugation polymer of Formula (IVc) is contacted with PAL under conditions effective to conjugate four or more, e.g., 4-21, pre-conjugation polymers of Formula (IVc) individually to four or more lysine side chains of each subunit of PAL.

In one embodiment, pre-conjugation polymer of Formula (IVc) is contacted with PAL under conditions effective to conjugate one or more, e.g., 1-5 or 1-6, linker-POEGMAs to one or more lysines on each subunit of PAL, wherein the one or more lysines on each subunit of PAL comprise independently (e.g., may be different for each subunit) one or more, e.g., 1-9, of K189, K195, K522, K413, K145, K32, K115, K335, and K109, although in some embodiments, one of ordinary skill in the art will understand that other lysine residues of PAL may also be conjugated to a linker-POEGMA. In one embodiment, pre-conjugation polymer of Formula (IVc) is contacted with PAL under conditions effective to conjugate two or more, e.g., 2-5 or 2-6, linker-POEGMAs each individually to two or more lysines on each subunit of PAL, wherein the two or more lysines on each subunit of PAL comprise independently (e.g., may be different for each subunit) two or more, e.g., 2-9, of K189, K195, K522, K413, K145, K32, K115, K335, and K109, although in some embodiments, one of ordinary skill in the art will understand that other lysine residues of PAL may also be conjugated to a linker-POEGMA. In one embodiment, a pre-conjugation polymer of Formula (IVc) is contacted with PAL under conditions effective to conjugate three or more, e.g., 3-5 or 3-6, linker-POEGMAs each individually to or three or more lysines on each subunit of PAL, wherein the three or more lysines on each subunit of PAL comprise independently (e.g., may be different for each subunit) three or more, e.g., 3-9, of K189, K195, K522, K413, K145, K32, K115, K335, and K109, although in some embodiments, one of ordinary skill in the art will understand that other lysine residues of PAL may also be conjugated to a linker-POEGMA. In one embodiment, pre-conjugation polymer of Formula (IVc) is contacted with PAL under conditions effective to conjugate four or more, e.g., 4-5 or 4-6, linker-POEGMAs each individually to four or more lysines on each subunit of PAL, wherein the four or more lysines on each subunit of PAL comprise independently (e.g., may be different for each subunit) four or more, e.g., 4-9, of K189, K195, K522, K413, K145, K32, K115, K335, and K109, although in some embodiments, one of ordinary skill in the art will understand that other lysine residues of PAL may also be conjugated to a linker-POEGMA. In one embodiment, pre-conjugation polymer of Formula (IVc) is contacted with PAL under conditions effective to conjugate five or more, e.g., 5 or 6, linker-POEGMAs each individually to five or more lysines on each subunit of PAL, wherein the five or more lysines on each subunit of PAL comprise independently (e.g., may be different for each subunit) five or more, e.g., 5-9, of K189, K195, K522, K413, K145, K32, K115, K335, and K109, although in some embodiments, one of ordinary skill in the art will understand that other lysine residues of PAL may also be conjugated to a linker-POEGMA.

In some embodiments, the conditions are effective to conjugate each of K189, K195 and K522 to a linker-POEGMA in at least one, e.g., 1-4, subunit of PAL. In some embodiments, the conditions are effective to conjugate each of K189, K195 and K522 to a linker-POEGMA in at least two subunits, e.g., 2-4, of PAL. In some embodiments, the conditions are effective to conjugate each of K189, K195 and K522 to a linker-POEGMA in at least three subunits, e.g., 3 or 4, of PAL. In some embodiments, the conditions are effective to conjugate each of K189, K195 and K522 to a linker-POEGMA in each subunit of PAL.

In some embodiments, pre-conjugation polymer of Formula (IVc) is contacted with PAL under conditions are effective to conjugate each of K189, K195, K522, K413, and K145 to a linker-POEGMA in at least one, e.g., 1-4, subunit of PAL. In some embodiments, the conditions are effective to conjugate each of K189, K195, K522, K413, and K145 to a linker-POEGMA in at least two, e.g., 2-4, subunits of PAL. In some embodiments, the conditions are effective to conjugate each of K189, K195, K522, K413, and K145 to a linker-POEGMA in at least three, e.g., 3 or 4, subunits of PAL. In some embodiments, the conditions are effective to conjugate each of K189, K195, K522, K413, and K145 to a linker-POEGMA in each subunit of PAL.

Conditions effective for conjugating the pre-conjugation polymer of Formula (IVc) containing a pentafluorophenyl ester conjugation moiety are well known to one of ordinary skill in the art. For example, a POEGMA-PAL conjugate of Formula (I) may be prepared by contacting PAL with a pre-conjugation polymer of Formula (IVc) in a solvent (e.g., an aqueous buffer) at a pH of about 7 to about 9.

In one embodiment, R₄ is

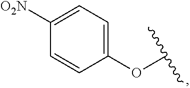

wherein [symbol] represents attachment to R₁ and a POEGMA-PAL conjugate of Formula (I) may be prepared by a method comprising contacting polyethylene glycol methacrylate with a compound of Formula (IId) to prepare a polymer of Formula (IIId), as shown in SCHEME Id below:

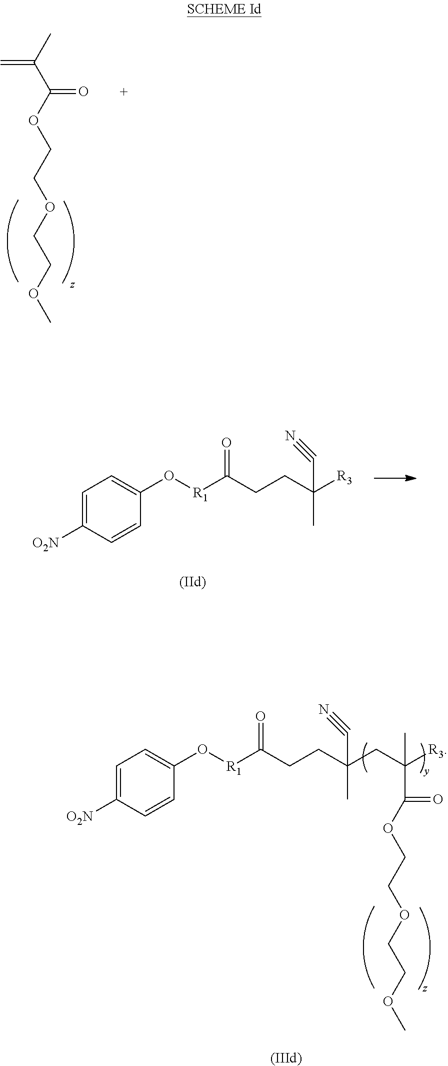

The polymer of formula (IIId) may then be contacted with an end group modification agent to remove R₃ to generate a pre-conjugation polymer of Formula (IVd) having end cap R₂, as shown in SCHEME IId below:

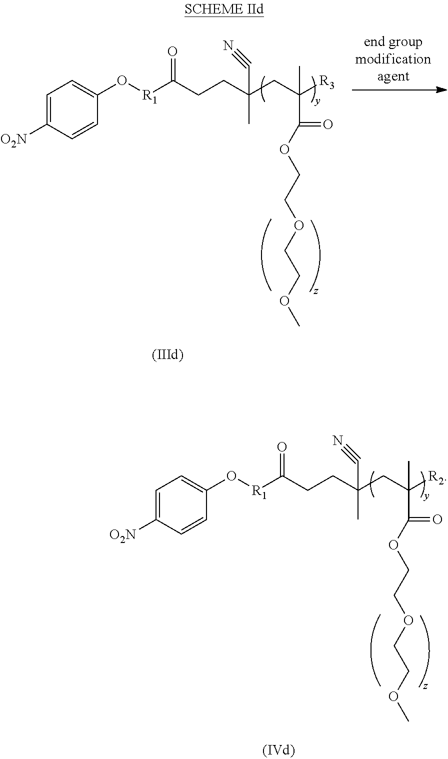

The pre-conjugation polymer of Formula (IVd) may then be contacted with PAL under conditions effective to conjugate the pre-conjugation polymer of Formula (IVd) to PAL, thereby generating a POEGMA-PAL conjugate of Formula (I), as shown in SCHEME IIId below:

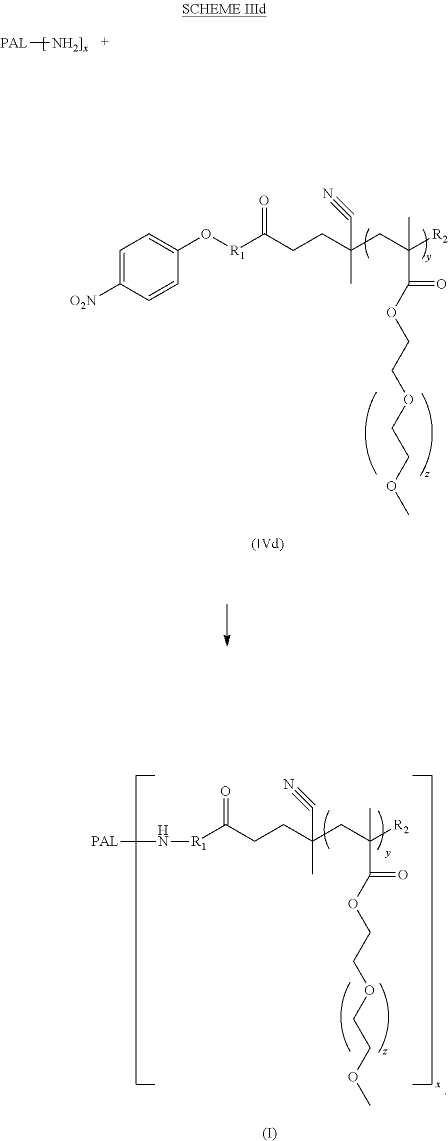

In each of SCHEMES Id, IId, and IIId:
PAL is a prokaryotic phenylalanine ammonia-lyase;
R₁ is a linker;
x is an integer from 1 to about 21;
y is an integer from about 50 to about 500; and
z is an integer from 2 to about 10.

In one embodiment, PAL is a wild-type PAL from a prokaryotic organism, including, but not limited to, PAL from Streptomyces maritimus, Nostoc punctiforme, Anabaena variabilis, Anacystis nidulans, Photorabdus luminescens TT01, and Streptomyces verticillatus. In one embodiment, PAL is a fragment, mutant, variant, or analog of a wild-type PAL enzyme that retains similar (e.g., at least 50%) or increased catalytic activity for phenylalanine. In one embodiment, PAL is a prokaryotic PAL from Anabaena variabilis (SEQ ID NO:1) or biologically active fragment, mutant, variant or analog thereof. In certain embodiments, PAL is an Anabaena variabilis PAL (AvPAL) variant. In one embodiment, PAL is an AvPAL variant wherein one or more amino acid residues have been substituted by another amino acid residue. In some embodiments, one or more cysteine residues of the AvPAL variant have been substituted by a serine residue. In some embodiments, the one or more cysteine residues of the AvPAL variant that have been substituted by one or more serine residues are selected from the group consisting of cysteine residues at positions 503 and 565. In one embodiment, each subunit of PAL has an amino acid sequence according to SEQ ID NO:2. In one embodiment, PAL has an amino acid sequence according to SEQ ID NO: 3. In one embodiment, each subunit of PAL has an amino acid sequence according to SEQ ID NO:4.

In certain embodiments, pre-conjugation polymer of Formula (IVd) is contacted with PAL under conditions effective to conjugate two or more pre-conjugation polymers of Formula (IVd) individually to two or more, e.g., 2-21, lysine side chains of PAL. In one embodiment, pre-conjugation polymer of Formula (IVd) is contacted with PAL under conditions effective to conjugate a pre-conjugation polymer of Formula (IVd) each individually to about 5 to about 21, about 8 to about 21, about 10 to about 21, about 16 to about 21, or about 19 to about 21 lysine side chains of PAL. In one embodiment, pre-conjugation polymer of Formula (IVd) is contacted with PAL under conditions effective to conjugate a pre-conjugation polymer of Formula (IVd) each individually to about 19 to about 21 lysine side chains of PAL.

In certain embodiments, pre-conjugation polymer of Formula (IVd) is contacted with PAL under conditions effective to conjugate one or more, e.g., 1-21 or 1-8, pre-conjugation polymers of Formula (IVd) individually to one or more lysine side chains of each subunit of PAL. In certain embodiments, pre-conjugation polymer of Formula (IVd) is contacted with PAL under conditions effective to conjugate two or more, e.g., 2-21 or 2-8, pre-conjugation polymers of Formula (IVd) individually to two or more lysine side chains of each subunit of PAL. In certain embodiments, pre-conjugation polymer of Formula (IVd) is contacted with PAL under conditions effective to conjugate three or more, e.g., 3-21 or 3-8, pre-conjugation polymers of Formula (IVd) individually to three or more lysine side chains of each subunit of PAL. In certain embodiments, pre-conjugation polymer of Formula (IVd) is contacted with PAL under conditions effective to conjugate four or more, e.g., 4-21 or 4-8, pre-conjugation polymers of Formula (IVd) individually to four or more lysine side chains of each subunit of PAL.

In one embodiment, pre-conjugation polymer of Formula (IVd) is contacted with PAL under conditions effective to conjugate one or more, e.g., 1-5 or 1-6, linker-POEGMAs to one or more lysines on each subunit of PAL, wherein the one or more lysines on each subunit of PAL comprise independently (e.g., may be different for each subunit) one or more, e.g., 1-9, of K189, K195, K522, K413, K145, K32, K115, K335, and K109, although in some embodiments, one of ordinary skill in the art will understand that other lysine residues of PAL may also be conjugated to a linker-POEGMA. In one embodiment, pre-conjugation polymer of Formula (IVd) is contacted with PAL under conditions effective to conjugate two or more, e.g., 2-5 or 2-6, linker-POEGMAs each individually to two or more lysines on each subunit of PAL, wherein the two or more lysines on each subunit of PAL comprise independently (e.g., may be different for each subunit) two or more, e.g., 2-9, of K189, K195, K522, K413, K145, K32, K115, K335, and K109, although in some embodiments, one of ordinary skill in the art will understand that other lysine residues of PAL may also be conjugated to a linker-POEGMA. In one embodiment, a pre-conjugation polymer of Formula (IVd) is contacted with PAL under conditions effective to conjugate three or more, e.g., 3-5 or 3-6, linker-POEGMAs each individually to three or more lysines on each subunit of PAL, wherein the three or more lysines on each subunit of PAL comprise independently (e.g., may be different for each subunit) three or more, e.g., 3-9, of K189, K195, K522, K413, K145, K32, K115, K335, and K109, although in some embodiments, one of ordinary skill in the art will understand that other lysine residues of PAL may also be conjugated to a linker-POEGMA. In one embodiment, pre-conjugation polymer of Formula (IVd) is contacted with PAL under conditions effective to conjugate four or more, e.g., 4-5 or 4-6, linker-POEGMAs each individually to four or more lysines on each subunit of PAL, wherein the four or more lysines on each subunit of PAL comprise independently (e.g., may be different for each subunit) four or more, e.g., 4-9, of K189, K195, K522, K413, K145, K32, K115, K335, and K109, although in some embodiments, one of ordinary skill in the art will understand that other lysine residues of PAL may also be conjugated to a linker-POEGMA. In one embodiment, pre-conjugation polymer of Formula (IVd) is contacted with PAL under conditions effective to conjugate five or more, e.g., 5 or 6, linker-POEGMAs each individually to five or more lysines on each subunit of PAL, wherein the five or more lysines on each subunit of PAL comprise independently (e.g., may be different for each subunit) four or more, e.g., 5-9, of K189, K195, K522, K413, K145, K32, K115, K335, and K109, although in some embodiments, one of ordinary skill in the art will understand that other lysine residues of PAL may also be conjugated to a linker-POEGMA.

In some embodiments, the conditions are effective to conjugate each of K189, K195 and K522 to a linker-POEGMA in at least one, e.g., 1-4, subunit of PAL. In some embodiments, the conditions are effective to conjugate each of K189, K195 and K522 to a linker-POEGMA in at least two subunits, e.g., 2-4, of PAL. In some embodiments, the conditions are effective to conjugate each of K189, K195 and K522 to a linker-POEGMA in at least three subunits, e.g., 3 or 4, of PAL. In some embodiments, the conditions are effective to conjugate each of K189, K195 and K522 to a linker-POEGMA in each subunit of PAL.

In some embodiments, pre-conjugation polymer of Formula (IVd) is contacted with PAL under conditions are effective to conjugate each of K189, K195, K522, K413, and K145 to a linker-POEGMA in at least one, e.g., 1-4, subunit of PAL. In some embodiments, the conditions are effective to conjugate each of K189, K195, K522, K413, and K145 to a linker-POEGMA in at least two, e.g., 2-4, subunits of PAL. In some embodiments, the conditions are effective to conjugate each of K189, K195, K522, K413, and K145 to a linker-POEGMA in at least three, e.g., 3 or 4, subunits of PAL. In some embodiments, the conditions are effective to conjugate each of K189, K195, K522, K413, and K145 to a linker-POEGMA in each subunit of PAL.

In any of the Formulas depicted in SCHEMES Ia, Ib, Ic, Id, IIa, IIb, IIc, IId, IIIa, IIIb, IIIc, and IIId, x, y and z may be as defined above for Formulas (I), (V), and (VI). Any combination of x, y, and z are encompassed by the present disclosure.

Conditions effective for conjugating the pre-conjugation polymer of Formula (IVd) containing a para-nitrophenyl carbonate conjugation moiety are well known to one of ordinary skill in the art. For example, a POEGMA-PAL conjugate of Formula (I) may be prepared by contacting PAL with a pre-conjugation polymer of Formula (IVd) in a solvent (e.g., an aqueous buffer) at a pH of about 7 to about 9.

### 5.3 Compositions of POEGMA-PAL Conjugates

As will be appreciated by one skilled in the art, a POEGMA-PAL conjugate as disclosed herein (e.g., in Section 5.1) is provided as a composition comprising a plurality of POEGMA-PAL conjugates, such as a POEGMA-PAL conjugate of any one of Formulas (I), (I′), (Ia), (Ia-1), and (Ia-1-1), and any of Formulas (Ia-1-11)-(Ia-1-46). In such a composition, x (the number of linker-POEGMAs conjugated to each PAL enzyme), y (the number of poly(ethylene)methyl ether methacrylate monomers present in each linker-POEGMA), and z (the number of ethylene glycol moieties present in each side chain of each linker-POEGMA) may vary from conjugate to conjugate. Further, within a single conjugate, y and z may vary, or in some embodiments, only y varies. As such, a composition comprising a plurality of POEGMA-PAL conjugates may be described by an average number of linker-POEGMAs conjugated to each PAL enzyme (xₐᵥₑ), an average number of poly(ethylene)methyl ether methacrylate monomers present in each linker-POEGMA (yₐᵥₑ), and an average number of ethylene glycol moieties present in each side chain of each linker-POEGMA (zₐᵥₑ). A composition comprising a plurality of POEGMA-PAL conjugates, as described herein (e.g., of Formula (I), (I′), (Ia), (Ia-1), or (Ia-1-1)), may be characterized by an xₐᵥₑ of about 1 to about 21, about 8 to about 21, about 16 to about 21, or about 19 to about 21. In one embodiment, xₐᵥₑ is about 19. In one embodiment, xₐᵥₑ is about 20. In one embodiment, xₐᵥₑ is about 21. A composition comprising a plurality of POEGMA-PAL conjugates may exhibit a yₐᵥₑ of about 50 to about 500, about 150 to about 300, or about 175 to about 200. A composition comprising a plurality of POEGMA-PAL conjugates may exhibit a zₐᵥₑ of about 2 to about 10, about 2 to about 8, or about 2 to about 5. In one embodiment, a composition comprising a plurality of POEGMA-PAL conjugates exhibits a zₐᵥₑ of about 2. In some embodiments, the standard deviation of zₐᵥₑ is 0 (e.g., zis 2 in all POEGMA-PAL conjugates of the composition).

In one embodiment, a composition comprising a plurality of POEGMA-PAL conjugates, as described herein (e.g., of Formula (I), (I′), (Ia), (Ia-1), or (Ia-1-1)), may be characterized by an xₐᵥₑ of about 1 to about 32, about 2 to about 32, about 3 to about 32, about 4 to about 32, about 5 to about 32, about 6 to about 32, about 7 to about 32, about 8 to about 32, about 9 to about 32, about 10 to about 32, about 11 to about 32, about 12 to about 32, about 13 to about 32, about 14 to about 32, about 15 to about 32, about 16 to about 32, about 17 to about 32, about 18 to about 32, about 19 to about 32, about 20 to about 32, about 21 to about 32, about 22 to about 32, about 23 to about 32, about 25 to about 32, about 26 to about 32, about 27 to about 32, about 28 to about 32, about 29 to about 32, about 20 to about 32, or about 31 to about 32.

In one embodiment, a composition comprising a plurality of POEGMA-PAL conjugates, as described herein (e.g., of Formula (I), (I′), (Ia), (Ia-1), or (Ia-1-1)), may be characterized by an xₐᵥₑ of about 1 to about 28, about 2 to about 28, about 3 to about 28, about 4 to about 28, about 5 to about 28, about 6 to about 28, about 7 to about 28, about 8 to about 28, about 9 to about 28, about 10 to about 28, about 11 to about 28, about 12 to about 28, about 13 to about 28, about 14 to about 28, about 15 to about 28, about 16 to about 28, about 17 to about 28, about 18 to about 28, about 19 to about 28, about 20 to about 28, about 21 to about 28, about 22 to about 28, about 23 to about 28, about 24 to about 28, about 25 to about 28, about 26 to about 28, or about 27 to about 28.

In one embodiment, a composition comprising a plurality of POEGMA-PAL conjugates, as described herein (e.g., of Formula (I), (I′), (Ia), (Ia-1), or (Ia-1-1)), may be characterized by an xₐᵥₑ of about 1 to about 24, about 2 to about 24, about 3 to about 24, about 4 to about 24, about 5 to about 24, about 6 to about 24, about 7 to about 24, about 8 to about 24, about 9 to about 24, about 10 to about 24, about 11 to about 24, about 12 to about 24, about 13 to about 24, about 14 to about 24, about 15 to about 24, about 16 to about 24, about 17 to about 24, about 18 to about 24, about 19 to about 24, about 20 to about 24, about 21 to about 24, about 22 to about 24, or about 23 to about 24.

In one embodiment, a composition comprising a plurality of POEGMA-PAL conjugates, as described herein (e.g., of Formula (I), (I′), (Ia), (Ia-1), or (Ia-1-1)), may be characterized by an xₐᵥₑ of about 1 to about 21, about 2 to about 21, about 3 to about 21, about 4 to about 21, about 5 to about 21, about 6 to about 21, about 7 to about 21, about 8 to about 21, about 9 to about 21, about 10 to about 21, about 11 to about 21, about 12 to about 21, about 13 to about 21, about 14 to about 21, about 15 to about 21, about 16 to about 21, about 17 to about 21, about 18 to about 21, or about 19 to about 21.

A composition comprising a plurality of POEGMA-PAL conjugates may exhibit a yₐᵥₑ of about 50 to about 500, about 150 to about 300, about 175 to about 250, or about 175 to about 200. A composition comprising a plurality of POEGMA-PAL conjugates may exhibit a zₐᵥₑ of about 2 to about 10, about 2 to about 9, about 2 to about 8, about 2 to about 7, about 2 to about 6, about 2 to about 5, about 2 to about 4, or about 2 to about 3. In one embodiment, a composition comprising a plurality of POEGMA-PAL conjugates exhibits a zₐᵥₑ of about 2 to about 5. In certain embodiments, a composition comprising a plurality of POEGMA-PAL conjugates exhibits a zₐᵥₑ of about 2. In some embodiments, the standard deviation of zₐᵥₑ is 0 (e.g., z is 2 in all POEGMA-PAL conjugates of the composition). Generally, the combinations mentioned herein for x, y, and z are also applicable to xₐᵥₑ, yₐᵥₑ, and zₐᵥₑ.

Each POEGMA-PAL conjugate in a composition comprising a plurality of POEGMA-PAL conjugates, as described herein (e.g., of Formula (I), (I′), (Ia), (Ia-1), or (Ia-1-1), or any of Formulas (Ia-1-11)-(Ia-1-46)), may be conjugated as described herein, e.g., in Section 5.1. Conjugation sites may vary between each individual POEGMA-PAL conjugate in the composition, as will be described in the EXAMPLES.

In one embodiment, each POEGMA-PAL conjugate in a composition independently comprises 5-32 PAL lysine residues conjugated to a linker-POEGMA. In one embodiment, each POEGMA-PAL conjugate in a composition independently comprises at least 5 PAL lysine residues, at least 8 PAL lysine residues, at least 12 PAL lysine residues, at least 16 PAL lysine residues, at least 20 PAL lysine residues, at least 24 PAL lysine residues, or at least 28 PAL lysine residues conjugated to a linker-POEGMA. In one embodiment, each POEGMA-PAL conjugate in a composition independently comprises 10-32, 10-28, or 10-24 PAL lysine residues conjugated to a linker-POEGMA. In one embodiment, each POEGMA-PAL conjugate in a composition independently comprises 16-32, 16-28, or 16-24 PAL lysine residues conjugated to a linker-POEGMA. In one embodiment, each POEGMA-PAL conjugate in a composition independently comprises 19-32, 19-28, or 19-24 PAL lysine residues conjugated to a linker-POEGMA. In one embodiment, each POEGMA-PAL conjugate in a composition independently comprises 4-7 lysine residues on each PAL subunit conjugated to a linker-POEGMA. In one embodiment, each POEGMA-PAL conjugate in a composition independently comprises 4-8 lysine residues on each PAL subunit conjugated to a linker-POEGMA. In one embodiment, each POEGMA-PAL conjugate in a composition independently comprises 5-7 lysine residues on each PAL subunit conjugated to a linker-POEGMA.

In one embodiment, each POEGMA-PAL conjugate in a composition independently comprises 5-10 PAL lysine residues conjugated to a linker-POEGMA. In one embodiment, each POEGMA-PAL conjugate in a composition independently comprises at least 10 PAL lysine residues conjugated to a linker-POEGMA. In one embodiment, each POEGMA-PAL conjugate in a composition independently comprises 10-16 or 10-21 PAL lysine residues conjugated to a linker-POEGMA. In one embodiment, each POEGMA-PAL conjugate in a composition independently comprises at least 16 PAL lysine residues conjugated to a linker-POEGMA. In one embodiment, each POEGMA-PAL conjugate in a composition independently comprises 16-21 PAL lysine residues conjugated to a linker-POEGMA. In one embodiment, each POEGMA-PAL conjugate in a composition independently comprises at least 19 PAL lysine residues conjugated to a linker-POEGMA. In one embodiment, each POEGMA-PAL conjugate in a composition independently comprises 19-21 PAL lysine residues conjugated to a linker-POEGMA. In one embodiment, each POEGMA-PAL conjugate in a composition independently comprises at least 4 lysine residues on each PAL subunit conjugated to a linker-POEGMA. In one embodiment, each POEGMA-PAL conjugate in a composition independently comprises at least 5 lysine residues on each PAL subunit conjugated to a linker-POEGMA. In one embodiment, each POEGMA-PAL conjugate in a composition independently comprises 4-6 lysine residues on each PAL subunit conjugated to a linker-POEGMA.

In one embodiment, each POEGMA-PAL conjugate in a composition independently comprises one or more (e.g., 1-5 or 1-6) lysine residues on one or more PAL subunits (e.g., may be different for each subunit) conjugated to a linker-POEGMA, wherein the at least one (e.g., 1-9) of the lysine residues is selected from the group consisting of K189, K195, K522, K413, K145, K32, K115, K335, and K109, although a person of skill in the art will understand that in some embodiments, other lysine residues of PAL may also be conjugated to a linker-POEGMA. In one embodiment, each POEGMA-PAL conjugate in a composition independently comprises two or more (e.g., 2-5 or 2-6) lysine residues on one or more PAL subunits (e.g., 1-4 PAL subunits) may be different for each subunit) conjugated to a linker-POEGMA, wherein the two or more lysine residues is selected from the group consisting of K189, K195, K522, K413, K145, K32, K115, K335, and K109, although a person of skill in the art will understand that in some embodiments, other lysine residues of PAL may also be conjugated to a linker-POEGMA. In one embodiment, each POEGMA-PAL conjugate in a composition independently comprises three or more (e.g., 3-5 or 3-6) lysine residues on one or more PAL subunits (e.g., may be different for each subunit) conjugated to a linker-POEGMA, wherein the three or more lysine residues is selected from the group consisting of K189, K195, K522, K413, K145, K32, K115, K335, and K109, although a person of skill in the art will understand that in some embodiments, other lysine residues of PAL may also be conjugated to a linker-POEGMA. In one embodiment, each POEGMA-PAL conjugate in a composition independently comprises four or more (e.g., 4, 5, or 6) lysine residues on one or more PAL subunits (e.g., may be different for each subunit) conjugated to a linker-POEGMA, wherein the four or more lysine residues is selected from the group consisting of K189, K195, K522, K413, K145, K32, K115, K335, and K109, although a person of skill in the art will understand that in some embodiments, other lysine residues of PAL may also be conjugated to a linker-POEGMA. In one embodiment, each POEGMA-PAL conjugate in a composition independently comprises five or more (e.g., 5 or 6) lysine residues on one or more PAL subunits (e.g., may be different for each subunit) conjugated to a linker-POEGMA, wherein the five or more lysine residues is selected from the group consisting of K189, K195, K522, K413, K145, K32, K115, K335, and K109, although a person of skill in the art will understand that in some embodiments, other lysine residues of PAL may also be conjugated to a linker-POEGMA.

In one embodiment, each POEGMA-PAL conjugate in a composition independently comprises six or more (e.g., 6-8) lysine residues on one or more PAL subunits (e.g., may be different for each subunit) conjugated to a linker-POEGMA, wherein the six or more lysine residues is selected from the group consisting of K189, K195, K522, K413, K145, K32, K115, K335, and K109, although a person of skill in the art will understand that in some embodiments, other lysine residues of PAL may also be conjugated to a linker-POEGMA.

In one embodiment, each POEGMA-PAL conjugate in a composition independently comprises seven or more (e.g., 7 or 8) lysine residues on one or more PAL subunits (e.g., may be different for each subunit) conjugated to a linker-POEGMA, wherein the seven or more lysine residues is selected from the group consisting of K189, K195, K522, K413, K145, K32, K115, K335, and K109, although a person of skill in the art will understand that in some embodiments, other lysine residues of PAL may also be conjugated to a linker-POEGMA.

In some embodiments, each of K189, K195 and K522 in at least one subunit of PAL are conjugated to a linker-POEGMA in at least 60% of the POEGMA-PAL conjugates in the composition. In some embodiments, each of K189, K195 and K522 in at least two subunits of PAL are conjugated to a linker-POEGMA in at least 60% of the POEGMA-PAL conjugates in the composition. In some embodiments, each of K189, K195 and K522 in at least three subunits of PAL are conjugated to a linker-POEGMA in at least 60% of the POEGMA-PAL conjugates in the composition. In some embodiments, each of K189, K195 and K522 in each subunit of PAL are conjugated to a linker-POEGMA in at least 60% of the POEGMA-PAL conjugates in the composition.

In some embodiments, each of K189, K195, K522, K413, and K145 in at least one subunit of PAL are conjugated to a linker-POEGMA in at least 60% of the POEGMA-PAL conjugates in the composition. In some embodiments, each of K189, K195, K522, K413, and K145 in at least two subunits of PAL are conjugated to a linker-POEGMA in at least 60% of the POEGMA-PAL conjugates in the composition. In some embodiments, each of K189, K195, K522, K413, and K145 in at least three subunits of PAL are conjugated to a linker-POEGMA in at least 60% of the POEGMA-PAL conjugates in the composition. In some embodiments, each of K189, K195, K522, K413, and K145 in each subunit of PAL are conjugated to a linker-POEGMA in at least 60% of the POEGMA-PAL conjugates in the composition.

In another aspect, a composition comprising a plurality of POEGMA-PAL conjugates may be characterized by molecular weight, such as number average molecular weight (Mₙ), weight average molecular weight (M_{w}), or peak molecular weight (Mₚ). One of ordinary skill in the art will be familiar with methods for measuring Mₙ, M_{w}, and Mₚ. For example, in one embodiment, Mₙ, M_{w}, and Mₚ are measured by gel permeation chromatography or size exclusion chromatography with multi-angle static light scattering (SEC-MALS). In one embodiment, a composition comprising a plurality of POEGMA-PAL conjugates as disclosed herein may have an Mₙ of about 1100 kDa to about 1300 kDa, such as about 1200 kDa to about 1300 kDa, as measured by SEC-MALS. In one embodiment, a composition comprising a plurality of POEGMA-PAL conjugates as disclosed herein may have an Mₙ of about 1200 kDa, as measured by SEC-MALS. In one embodiment, a composition comprising a plurality of POEGMA-PAL conjugates as disclosed herein may have an M_{w} of about 1100 kDa to about 1300 kDa, such as about 1200 kDa to about 1300 kDa, as measured by SEC-MALS. In one embodiment, a composition comprising a plurality of POEGMA-PAL conjugates as disclosed herein may have an M_{w} of about 1200 kDa, as measured by SEC-MALS.

In one embodiment, a composition comprising a plurality of POEGMA-PAL conjugates as disclosed herein may have an Mₙ of about 1100 kDa to about 1700 kDa, such as about 1100 kDa to about 1300 kDa, about 1100 kDa to about 1400 kDa, about 1100 kDa to about 1500 kDa, or about 1100 kDa to about 1600 kDa, as measured by SEC-MALS. In one embodiment, a composition comprising a plurality of POEGMA-PAL conjugates as disclosed herein may have an Mₙ of about 1100 kDa, as measured by SEC-MALS. In one embodiment, a composition comprising a plurality of POEGMA-PAL conjugates as disclosed herein may have an M_{w} of about 1200 kDa, as measured by SEC-MALS. In one embodiment, a composition comprising a plurality of POEGMA-PAL conjugates as disclosed herein may have an M_{w} of about 1300 kDa, as measured by SEC-MALS. In one embodiment, a composition comprising a plurality of POEGMA-PAL conjugates as disclosed herein may have an M_{w} of about 1400 kDa, as measured by SEC-MALS. In one embodiment, a composition comprising a plurality of POEGMA-PAL conjugates as disclosed herein may have an M_{w} of about 1500 kDa, as measured by SEC-MALS. In one embodiment, a composition comprising a plurality of POEGMA-PAL conjugates as disclosed herein may have an M_{w} of about 1600 kDa, as measured by SEC-MALS. In one embodiment, a composition comprising a plurality of POEGMA-PAL conjugates as disclosed herein may have an M_{w} of about 1700 kDa, as measured by SEC-MALS.

In another aspect, a composition comprising a plurality of POEGMA-PAL conjugates may be characterized by its polydispersity index (PDI), which is the weight average molecular weight (M_{w}) of a composition divided by the number average molecular weight (Mₙ) of a composition. Advantageously, when a pre-conjugation polymer (e.g., Formula (IV) is prepared using RAFT, the resulting composition of pre-conjugation polymers has an M_{w} that is substantially the same as its Mₙ, thereby having a PDI of about 1. Accordingly, upon conjugation of POEGMA to PAL, a composition comprising a plurality of POEGMA-PAL conjugates may be characterized by a PDI of about 1. In certain embodiments, a composition comprising a plurality of POEGMA-PAL conjugates may be characterized by a PDI of about 1 to about 1.1. In certain embodiments, a composition comprising a plurality of POEGMA-PAL conjugates may be characterized by a PDI of about 1 to about 1.05. Without wishing to be bound by theory, it is believed that preparation of the POEGMA-PAL compositions according to the methods disclosed herein, e.g., RAFT polymerization, such as RAFT photopolymerization, allows a high degree of control over the polymerization process, and in particular, the degree of polymerization (e.g., the value of y in Formulas (I), (I′), (Ia), (Ia-1), (Ia-1-1), (III), (IV), and (V)).

### 5.4 Pharmaceutical Compositions of POEGMA-PAL Conjugates

The POEGMA-PAL conjugates as described herein may be used to treat a disease, condition, or disorder in a subject in need thereof. Therefore, in another aspect, the present disclosure provides a pharmaceutical composition comprising a therapeutically effective amount of a composition, as described in any embodiment in Section 5.3, comprising a plurality of POEGMA-PAL conjugates of Formula (I):

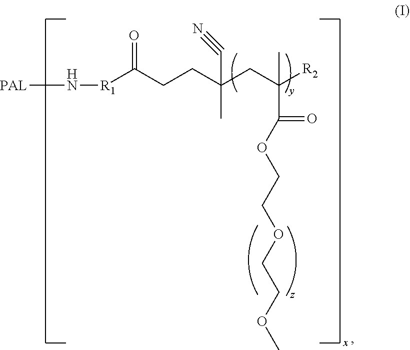

wherein:
PAL is a prokaryotic phenylalanine ammonia-lyase;
R₁ is a linker;
R₂ is an end cap;
x is an integer from 1 to about 21;
y is an integer from about 50 to about 500; and
z is an integer from 2 to about 10,
   and one or more pharmaceutically acceptable excipients.

In another embodiment, the present disclosure provides a pharmaceutical composition comprising a therapeutically effective amount of a composition, as described in any embodiment in Section 5.3, comprising a plurality of POEGMA-PAL conjugates of Formula (I):

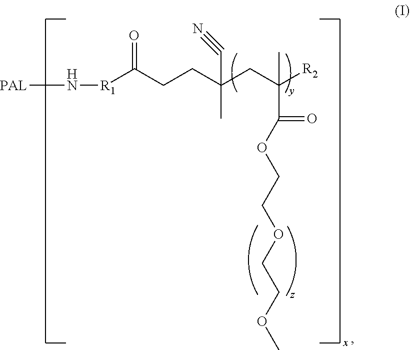

wherein:
PAL is a prokaryotic phenylalanine ammonia-lyase;
R₁ is a linker;
R₂ is an end cap;
x is an integer from 1 to about 32;
y is an integer from about 50 to about 500; and
z is an integer from 2 to about 10,
   and one or more pharmaceutically acceptable excipients.

In Formula (I), linker R₁ may be CH₂, C(O)—C₁₋₁₂ alkyl-NH, C(O)—(O—CH₂—CH₂) n, wherein n is 2 or 3, or a peptide cleavable spacer, such as Gly-Gly-Phe-Gly (SEQ ID NO:5). In one embodiment, R₁ is C(O)—C₁₋₁₂ alkyl-NH.

In one embodiment, R₁ is C(O)—C₁₋₁₂ alkyl-NH. In one embodiment, the pharmaceutical composition comprises a therapeutically effective amount of a composition, as described in any embodiment in Section 5.3, comprising a plurality of POEGMA-PAL conjugates of Formula (I′):

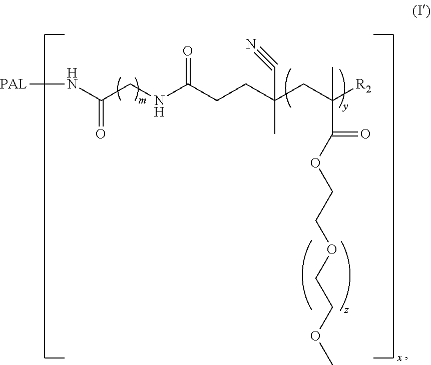

wherein:
PAL is a prokaryotic phenylalanine ammonia-lyase;
R₂ is an end cap;
m is an integer from about 3 to about 12;
x is an integer from 1 to about 21;
y is an integer from about 50 to about 500; and
z is an integer from 2 to about 10.

In another embodiments, the pharmaceutical composition comprises a therapeutically effective amount of a composition, as described in any embodiment in Section 5.3, comprising a plurality of POEGMA-PAL conjugates of Formula (I′):

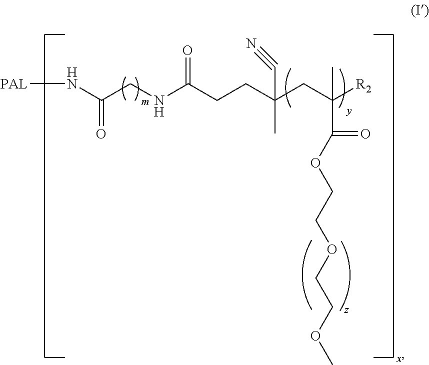

wherein:
PAL is a prokaryotic phenylalanine ammonia-lyase;
R₂ is an end cap;
m is an integer from about 3 to about 12;
x is an integer from 1 to about 32;
y is an integer from about 50 to about 500; and
z is an integer from 2 to about 10.

In any of the above Formulae, m is 3, 4, 5, 6, 7, 8, 9, 10, 11, or 12, such as an integer from 3 to 10, 3 to 8 or 3 to 5. In one embodiment, m is 5.

In one embodiment, R₁ is C(O)—(CH₂)₅—NH. In one embodiment, the pharmaceutical composition comprises a therapeutically effective amount of a composition, as described in any embodiment in Section 5.3, comprising a plurality of POEGMA-PAL conjugates of Formula (Ia):

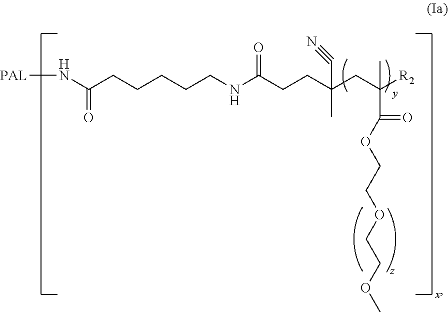

wherein:
PAL is a prokaryotic phenylalanine ammonia-lyase;
R₂ is an end cap;
x is an integer from 1 to about 21;
y is an integer from about 50 to about 500; and
z is an integer from 2 to about 10.

In another embodiment, the pharmaceutical composition comprises a therapeutically effective amount of a composition, as described in any embodiment in Section 5.3, comprising a plurality of POEGMA-PAL conjugates of Formula (Ia):

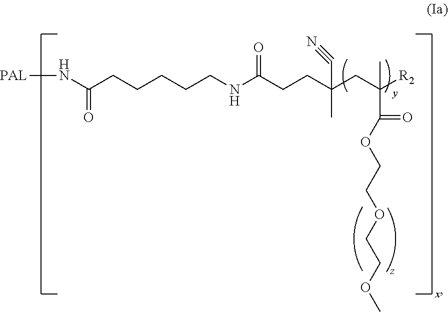

wherein:
PAL is a prokaryotic phenylalanine ammonia-lyase;
R₂ is an end cap;
x is an integer from 1 to about 32;
y is an integer from about 50 to about 500; and
z is an integer from 2 to about 10.

In one embodiment of Formula (I), (I′), or (Ia), R₂ is isobutyronitrile, 4-cyanovaleric acid, 4-methoxy-2,4-dimethylvaleronitrile, or a hydroxy group (—OH). In one embodiment, R₂ is isobutyronitrile.

In one embodiment, the pharmaceutical composition comprises a therapeutically effective amount of a composition, as described in any embodiment in Section 5.3, comprising a plurality of POEGMA-PAL conjugates of Formula (Ia-1):

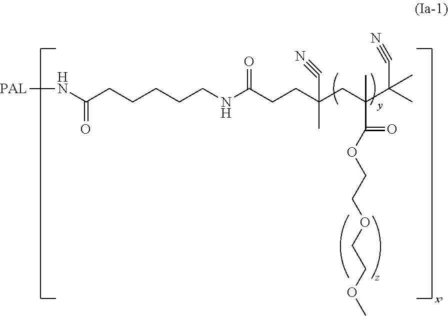

wherein:
PAL is a prokaryotic phenylalanine ammonia-lyase;
x is an integer from 1 to about 21;
y is an integer from about 50 to about 500; and
z is an integer from 2 to about 10.

In another embodiment, the pharmaceutical composition comprises a therapeutically effective amount of a composition, as described in any embodiment in Section 5.3, comprising a plurality of POEGMA-PAL conjugates of Formula (Ia-1):

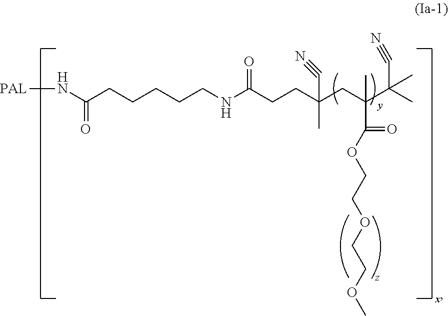

wherein:
PAL is a prokaryotic phenylalanine ammonia-lyase;
x is an integer from 1 to about 32;
y is an integer from about 50 to about 500; and
z is an integer from 2 to about 10.

In one embodiment of Formula (I), (I′), (Ia), or (Ia-1), z is 2 to about 10, such as 2 to about 8 or 2 to about 5. In one embodiment, z is 2. In one embodiment, the pharmaceutical composition comprises a therapeutically effective amount of a composition, as described in any embodiment in Section 5.3, comprising a plurality of POEGMA-PAL conjugates of Formula (Ia-1-1):

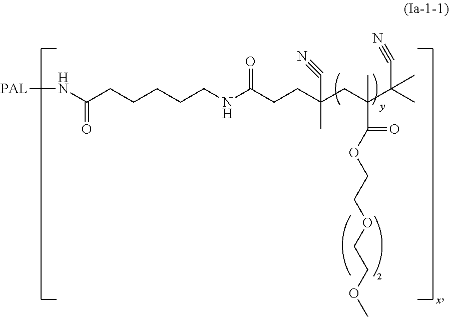

wherein:
PAL is a prokaryotic phenylalanine ammonia-lyase;
x is an integer from 1 to about 21; and
y is an integer from about 50 to about 500.

In another embodiment, the pharmaceutical composition comprises a therapeutically effective amount of a composition, as described in any embodiment in Section 5.3, comprising a plurality of POEGMA-PAL conjugates of Formula (Ia-1-1):

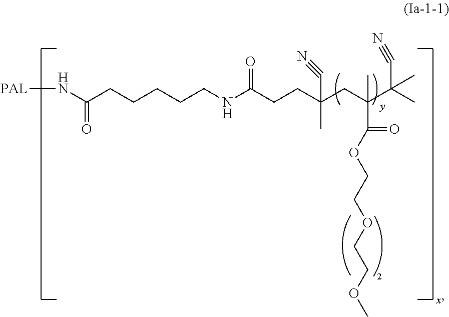

wherein:
PAL is a prokaryotic phenylalanine ammonia-lyase;
x is an integer from 1 to about 32; and
y is an integer from about 50 to about 500.

In one embodiment of Formula (I), (I′), (Ia), (Ia-1), or (Ia-1-1), or any of Formula (Ia-1-11)-(Ia-1-46), PAL is a wild-type PAL from a prokaryotic organism, including, but not limited to, PAL from Streptomyces maritimus, Nostoc punctiforme, Anabaena variabilis, Anacystis nidulans, Photorabdus luminescens TT01, and Streptomyces verticillatus. In one embodiment, PAL is a fragment, mutant, variant, or analog of a wild-type PAL enzyme that retains similar (e.g., at least 50%) or increased catalytic activity for phenylalanine. In one embodiment, PAL is a prokaryotic PAL from Anabaena variabilis (SEQ ID NO:1) or biologically active fragment, mutant, variant or analog thereof. In certain embodiments, PAL is an Anabaena variabilis PAL (AvPAL) variant. In one embodiment, PAL is an AvPAL variant wherein one or more amino acid residues have been substituted by another amino acid residue. In some embodiments, one or more cysteine residues of the AvPAL variant have been substituted by a serine residue. In some embodiments, the one or more cysteine residues of the AvPAL variant that have been substituted by one or more serine residues are selected from the group consisting of cysteine residues at positions 503 and 565. In one embodiment, each subunit of PAL has an amino acid sequence according to SEQ ID NO:2. In one embodiment, each subunit of PAL has an amino acid sequence according to SEQ ID NO:3. In one embodiment, each subunit of PAL has an amino acid sequence according to SEQ ID NO: 4.

x, y, and z may be as defined above for Formulas (I), (V), and (VI). In one embodiment of Formulas (I′), (Ia), (Ia-1), and (Ia-1-1), x is an integer from about 5 to about 32, about 8 to about 32, about 10 to about 32, about 16 to about 32, about 19 to about 32, about 24 to about 32, or about 28 to about 32. In one embodiment of Formulas (I′), (Ia), (Ia-1), and (Ia-1-1), x is an integer from about 5 to about 28, about 8 to about 28, about 10 to about 28, about 16 to about 28, about 19 to about 28, about 24 to about 28. In one embodiment of Formulas (I′), (Ia), (Ia-1), and (Ia-1-1), x is an integer from about 5 to about 24, about 8 to about 24, about 10 to about 24, about 16 to about 24, or about 19 to about 24. In one embodiment, y is an integer from about 50 to about 500, such as about 150 to about 300 or about 175 to about 200. In one embodiment, z is an integer from 2 to about 10, such as 2 to about 8 or 2 to about 5. In one embodiment, z is 2. Any combination of x, y, and z is encompassed by the present disclosure.

In any embodiment of Formulas (I), (I′), (Ia), (Ia-1), and (Ia-1-1), x may be an integer from 5 to about 21, such as about 8 to about 21, about 10 to about 21, about 16 to about 21, or about 19 to about 21. In any embodiment, y may be an integer from about 50 to about 500, such as about 150 to about 300 or about 175 to about 200. In any embodiment of Formulas (I), (I′), (Ia), and (Ia-1), z may be about 2 to about 10, about 2 to about 8, or 2 to about 5. In one embodiment, z is 2. Any combination of x, y, and z is encompassed by the present disclosure.

A pharmaceutical composition comprising a therapeutically effective amount of a composition, as described in any embodiment in Section 5.3, comprising a plurality of POEGMA-PAL conjugates, may comprise any pharmaceutically acceptable excipient or combination of excipients. Non-limiting examples of pharmaceutically acceptable excipients that may be included in a pharmaceutical composition together with a plurality of POEGMA-PAL conjugates as disclosed herein include liquids, such as water, saline, glycerol, polyethylene glycol, hyaluronic acid, ethanol, cyclodextrins, modified cyclodextrins, and the like. Suitable excipients for non-liquid formulations are also known to those of skill in the art.

Auxiliary agents, such as wetting or emulsifying agents, buffer, surfactants, and the like, may be present in a pharmaceutical composition comprising the plurality of POEGMA-PAL conjugates. A buffer can be solution that is pharmacologically acceptable, and which imparts to the pharmaceutical composition a desired pH (e.g., a pH in a physiologically acceptable range). Examples of buffer solutions include, but are not limited to, saline, phosphate buffered saline, Tris buffered saline, Hank's buffered saline, and the like.

Depending on the intended mode of administration, a pharmaceutical composition comprising the plurality of POEGMA-PAL conjugates may be in the form of a liquid dosage form, such as a liquid, solution, suspension, cream, ointment, lotion, or the like. In certain embodiments, the dosage form is a unit dosage form suitable for single administration of a precise dose of the POEGMA-PAL conjugate. In certain embodiments, the pharmaceutical composition is in the form of a liquid. In certain embodiments, the pharmaceutical composition is in the form of a suspension.

For parenteral administration, the composition will generally take the form of a liquid solution or suspension, solid or lyophilized form suitable for reconstitution, solubilization or suspension in liquid prior to injection, or as an emulsion. Sterile injectable suspensions may be formulated according to techniques known in the art using suitable carriers, dispersing, or wetting agents and suspending agents. Sterile injectable formulation may also be a sterile injectable solution or a suspension in a nontoxic parenterally acceptable diluent or solvent. Among the acceptable vehicles and solvents that may be employed are water, Ringer's solution, and isotonic sodium chloride solution. In addition, sterile, fixed oils, fatty esters, or polyols are conventionally employed as solvents or suspending media. In addition, parenteral administration may involve the use of a slow release or sustained release system.

A pharmaceutical composition comprising POEGMA-PAL conjugates may comprise a buffering agent to maintain the pH of the pharmaceutical composition, e.g., solution, within a desired range. Non-limiting examples of suitable buffering agents include tris-HCl, sodium acetate, sodium phosphate, and sodium citrate. Mixtures of these buffering agents may also be used. The amount of buffering agent useful in the pharmaceutical composition depends largely on the particular buffer used and the pH of the pharmaceutical composition, e.g., solution. In one embodiment, the pharmaceutical composition has a pH of about pH 6.0-8.5. In one embodiment, the pharmaceutical composition has a pH of about pH 7.0-8.0. In one embodiment, the pharmaceutical composition has a pH of about pH 7.0-7.6.

Optionally, a pharmaceutical composition can comprise one or more stabilizers, which are molecules that stabilize the POEGMA-PAL compositions of the disclosure. The term “stabilize” or “stabilizes” as used herein, is meant to include, for example and not for limitation, increasing the shelf-life of a composition of POEGMA-PAL conjugates, protecting the PAL enzyme of a composition of POEGMA-PAL conjugates from proteolytic digestion, maintaining the PAL enzyme of a composition of POEGMA-PAL conjugates in an active conformation, and preserving the PAL enzyme activity upon storage at elevated temperatures. Suitable stabilizers that may be included in the pharmaceutical compositions disclosed herein include, but are not limited to, L-phenylalanine (Phe) and structural analogs thereof, such as trans-cinnamic acid (t-CA), benzoic acid, tyrosine (Tyr), and the like.

In one embodiment, the POEGMA-PAL pharmaceutical compositions of the present disclosure comprise a plurality of POEGMA-PAL conjugates according to any embodiment disclosed herein (e.g., Formula (I), (I′), (Ia), (Ia-1), or (Ia-1-1), or any of Formula (Ia-1-11)-(Ia-1-46)) is provided as a liquid composition. In one embodiment, the POEGMA-PAL pharmaceutical compositions of the present disclosure comprise a plurality of POEGMA-PAL conjugates according to any embodiment disclosed herein (e.g., Formula (I), (I′), (Ia), (Ia-1), or (Ia-1-1), or any of Formula (Ia-1-11)-(Ia-1-46)) is provided as a solution for injection.

In certain embodiments, the POEGMA-PAL pharmaceutical compositions of the present disclosure comprise a plurality of POEGMA-PAL conjugates according to any embodiment disclosed herein (e.g., Formula (I), (I′), (Ia), (Ia-1), (Ia-1-1), or any of (Ia-1-11)-(Ia-1-46)), sodium chloride, trans-cinnamic acid, tromethamine, and tromethamine hydrochloride.

As discussed herein and as will be demonstrated in the Examples, pharmaceutical compositions of the POEGMA-PAL conjugates described herein (e.g., Formula (I), (I′), (Ia), (Ia-1), or (Ia-1-1), or any of Formula (Ia-1-11)-(Ia-1-46)) may be prepared at high concentrations while maintaining a low viscosity, thereby providing a mechanism by which higher doses can be provided to a subject via injection, particularly via injection routes where volume is limited (e.g., formulated for subcutaneous or intramuscular administration). For example, in one embodiment, a pharmaceutical composition comprising 20 mg/mL or less of a plurality of POEGMA-PAL conjugates (in tris buffer (20 mM with 136 mM NaCl) exhibits a viscosity of 10 cP or less. In one embodiment, a pharmaceutical composition comprising 20 mg/mL or less of a plurality of POEGMA-PAL conjugates exhibits a viscosity of 1 cP to 10 cP. In one embodiment, a pharmaceutical composition comprising 20 mg/mL to 60 mg/mL of a plurality of POEGMA-PAL conjugates (in tris buffer (20 mM with 136 mM NaCl) exhibits a viscosity of 70 cP or less. In one embodiment, a pharmaceutical composition comprising 20 mg/mL to 60 mg/mL of a plurality of POEGMA-PAL conjugates exhibits a viscosity of 1 cP to 70 cP. Viscosity may be measured by any known method in the art, for example, using a viscometer.

As such, provided herein are pharmaceutical compositions comprising a plurality of POEGMA-PAL conjugates of Formula (I), (I′), (Ia), (Ia-1), or (Ia-1-1), or any of Formula (Ia-1-11)-(Ia-1-46) as disclosed herein and at least one pharmaceutically acceptable excipient. In one embodiment, a pharmaceutical composition comprises about 20 mg/mL to about 60 mg/mL of a plurality of POEGMA-PAL conjugates of Formula (I), (I′), (Ia), (Ia-1), or (Ia-1-1), or any of Formula (Ia-1-11)-(Ia-1-46) as disclosed herein and at least one pharmaceutically acceptable excipient. In one embodiment, the pharmaceutically acceptable excipient is an aqueous buffer. In one embodiment, the buffer is Tris buffer. In certain embodiments, the pharmaceutical composition comprises about 20 mg/mL to about 60 mg/mL of a plurality of POEGMA-PAL conjugates of Formula (I), (I′), (Ia), (Ia-1), or (Ia-1-1), or any of Formula (Ia-1-11)-(Ia-1-46) as disclosed herein and at least one pharmaceutically acceptable excipient and has a viscosity of about 1 cP to about 70 cP, such as about 5 cP to about 70 cP. In certain embodiments, the pharmaceutical composition comprises about 20 mg/mL to about 60 mg/mL of a plurality of POEGMA-PAL conjugates of Formula (I), (I′), (Ia), (Ia-1), (Ia-1-1), or any of (Ia-1-11)-(Ia-1-46) as disclosed herein and at least one pharmaceutically acceptable excipient and has a viscosity of about 4 cP to about 65 cP. In certain embodiments, the pharmaceutical composition comprises about 20 mg/mL to about 40 mg/mL of a plurality of POEGMA-PAL conjugates of (I), (I′), (Ia), (Ia-1), or (Ia-1-1), or any of Formula (Ia-1-11)-(Ia-1-46) as disclosed herein and at least one pharmaceutically acceptable excipient and has a viscosity of about 5 cP to about 20 cP.

In one embodiment, the pharmaceutical composition is provided as a unit dosage form, wherein the unit dosage form contains about 0.5 mL to about 1.5 mL of a pharmaceutical composition comprising about 20 mg/mL to about 60 mg/mL of a plurality of POEGMA-PAL conjugates of Formula (I), (I′), (Ia), (Ia-1), or (Ia-1-1), or any of Formula (Ia-1-11)-(Ia-1-46) as disclosed herein and at least one pharmaceutically acceptable excipient. In one embodiment, the pharmaceutical composition is provided in a kit comprising one or more unit dosage forms containing about 0.5 mL to about 1.5 mL of a pharmaceutical composition comprising about 20 mg/mL to about 60 mg/mL of a plurality of POEGMA-PAL conjugates of Formula (I), (I′), (Ia), (Ia-1), or (Ia-1-1), or any of Formula (Ia-1-11)-(Ia-1-46) as disclosed herein and at least one pharmaceutically acceptable excipient and a means for administering (e.g., injecting) the pharmaceutical composition to a subject. In one embodiment, the means for administering is a syringe. In one embodiment, the syringe comprises 0.5 mL to 1.5 mL of the pharmaceutical composition.

In one embodiment, the POEGMA-PAL pharmaceutical composition comprises a plurality of POEGMA-PAL conjugates of any one of Formula (I), (I′), (Ia), (Ia-1), and (Ia-1-1), and any of Formula (Ia-1-11)-(Ia-1-46) is provided as a liquid composition, e.g., suspension or solution. In one embodiment, the POEGMA-PAL pharmaceutical composition comprises a plurality of POEGMA-PAL conjugates of any one of Formula (I), (I′), (Ia), (Ia-1), and (Ia-1-1), and any of Formula (Ia-1-11)-(Ia-1-46) is provided as a solution for injection. In one embodiment, the injection is subcutaneous injection. In one embodiment, the POEGMA-PAL pharmaceutical composition comprises a plurality of POEGMA-PAL conjugates of any one of Formula (I), (I′), (Ia), (Ia-1), and (Ia-1-1), and any of Formula (Ia-1-11)-(Ia-1-46) at a concentration of about 20 mg/mL. In one embodiment, the POEGMA-PAL pharmaceutical composition comprises a plurality of POEGMA-PAL conjugates of any one of Formulas (Ia-1-11)-(Ia-1-46) at a concentration of about 20 mg/mL.

### 5.5 Therapeutic Uses of POEGMA-PAL Conjugates

Advantageously, and as will be demonstrated in the EXAMPLES, the POEGMA-PAL conjugates disclosed herein, comprising PAL and a plurality of bottlebrush polymer linker-POEGMAs of Formula (V) conjugated thereto, appear to cause a lower immune response than other known PAL-based therapies, such as Pegvaliase. POEGMA-PAL conjugates and pharmaceutical compositions thereof additionally demonstrate improved pharmacokinetics (e.g., faster Tₘₐₓ) than other known PAL-based therapies, such as Pegvaliase. Yet further, pharmaceutical compositions of the POEGMA-PAL conjugates disclosed herein can be formulated at higher concentrations than other known PAL-based therapies, such as Pegvaliase, and still exhibits suitable viscosity for administration via methods where injection volume is limited, e.g., subcutaneous and intramuscular injection.

PKU is a disorder marked by a deficiency in PAH. One of ordinary skill in the art will understand that PAH deficiency includes PKU of varying severity and hyperphenylalaninemia (HPA) (Camp et al., Mol Genet Metab. 112 (2): 87-122 (2014); Kayaalp et al., Am J Hum Genet. 61:1309-17 (1997); and Guldberg et al., Am J Hum Genet. 63:71-9 (1998), each of which are incorporated herein by reference in their entirety), which are disorders that prevent the body from processing phenylalanine, an essential amino acid found in all protein-containing foods due to reduced or lack of activity by the hepatic enzyme PAH, which converts phenylalanine to tyrosine. PAH deficiency includes disorders characterized by a complete or near-complete deficiency of PAH activity in which a subject can tolerate less than 20 mg/kg/day of dietary phenylalanine to keep blood concentration of phenylalanine at a safe level of ≤300 μmol/L (e.g., classic PKU) as well as less severe forms having higher tolerance levels, such moderate PKU, e.g., having a tolerance of about 45-50 mg/kg/day of dietary phenylalanine, mild PKU, e.g., having a tolerance of about 55 mg/kg/day of dietary phenylalanine, or mild hyperphenylalaninemia (HPA)-gray zone, e.g., having a tolerance of about 70 mg/kg/day of dietary phenylalanine.

In one aspect, disclosed herein is a method of treating PAH deficiency in a subject, the method comprising administering to the subject a pharmaceutical composition, e.g., comprising a composition according to any embodiment in Section 5.3, which comprises a therapeutically effective amount of a plurality of POEGMA-PAL conjugates of Formula (I):

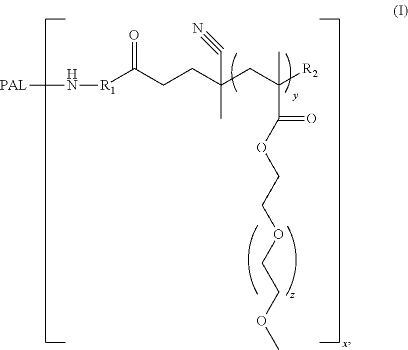

wherein:
PAL is a prokaryotic phenylalanine ammonia-lyase;
R₁ is a linker;
R₂ is an end cap;
x is an integer from 1 to about 32;
y is an integer from about 50 to about 500; and
z is an integer from 2 to about 10.

In one embodiment, linker R₁ is C(O)—C₁₋₁₂ alkyl-NH. In one embodiment, R₁ is C(O)—(CH₂)₅—NH. Accordingly, in one embodiment, the method of treating PAH deficiency in a subject comprises administering to the subject a pharmaceutical composition, e.g., comprising a composition according to any embodiment in Section 5.3, which comprises a therapeutically effective amount of a plurality of POEGMA-PAL conjugates of Formula (Ia):

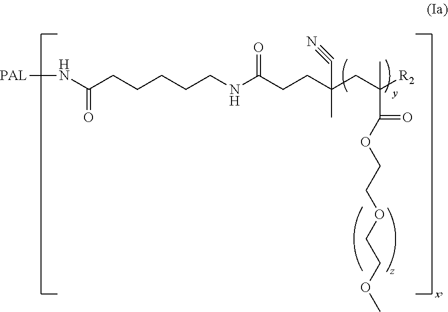

wherein:
PAL is a prokaryotic phenylalanine ammonia-lyase;
R₂ is an end cap;
x is an integer from 1 to about 32;
y is an integer from about 50 to about 500; and
z is an integer from 2 to about 10.

In one embodiment, end cap R₂ is 4-cyanovaleric acid, 4-methoxy-2,4-dimethylvaleronitrile, or hydroxy (—OH). In one embodiment, R₂ is isobutyronitrile. Accordingly, in one embodiment, the method of treating PAH deficiency in a subject comprises administering to the subject a pharmaceutical composition, e.g., comprising a composition according to any embodiment in Section 5.3, which comprises a therapeutically effective amount of a plurality of POEGMA-PAL conjugates of Formula (Ia-1):

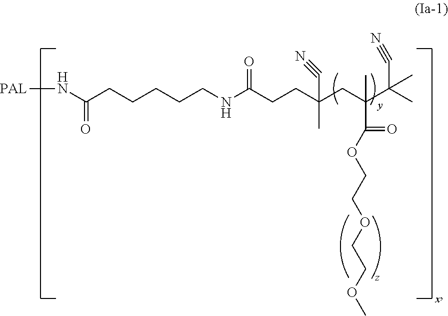

wherein:
PAL is a prokaryotic phenylalanine ammonia-lyase;
x is an integer from 1 to about 32;
y is an integer from about 50 to about 500; and
z is an integer from 2 to about 10.

In one embodiment, z is 2 to about 10. In one embodiment, z is an integer from 2 to about 8, such as 2 to about 5. In certain embodiments, z is 2.

In one embodiment, the method of treating PAH deficiency in a subject comprises administering to the subject a pharmaceutical composition, e.g., comprising a composition according to any embodiment in Section 5.3, which comprises a therapeutically effective amount of a plurality of POEGMA-PAL conjugates of Formula (Ia-1-1):

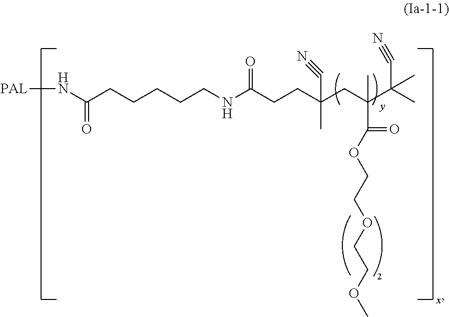

wherein:
PAL is a prokaryotic phenylalanine ammonia-lyase;
x is an integer from 1 to about 32; and
y is an integer from about 50 to about 500.

x and y may be as defined above for Formulas (I), (V), and (VI). In certain embodiments of Formulas (I), (I′), (Ia), (Ia-1), and (Ia-1-1), x may be an integer from about 5 to about 32, about 8 to about 32, about 10 to about 32, about 16 to about 32, about 19 to about 32, about 24 to about 32, about 28 to about 32, about 5 to about 28, about 8 to about 28, about 10 to about 28, about 16 to about 28, about 19 to about 28, about 24 to about 28, about 5 to about 24, about 8 to about 24, about 10 to about 24, about 16 to about 24, about 19 to about 24, about 5 to about 21, about 8 to about 21, about 10 to about 21, about 16 to about 21, or about 19 to about 21. In one embodiment, x is about 19. In one embodiment, x is about 20. In one embodiment, x is about 21. In one embodiment, y is an integer from about 150 to about 300, or about 175 to about 200. Any combination of x and y is encompassed by the present disclosure.

In one embodiment, the method of treating PAH deficiency in a subject comprises administering to the subject a pharmaceutical composition, e.g., comprising a composition according to any embodiment in Section 5.3, which comprises a therapeutically effective amount of a plurality of POEGMA-PAL conjugates of one or more of Formulas (Ia-1-11)-(Ia-1-46) shown in Table A.

In one embodiment of using any one of Formulas (I), (I′), (Ia), (Ia-1), and (Ia-1-1) or any one of Formulas (Ia-1-11)-(Ia-1-46) to treat PAH deficiency, the subject has PKU or HPA. In some embodiments, the subject has a blood phenylalanine level of about 300 μmol/L to about 350 μmol/L. In some embodiments, the subject has a blood phenylalanine level of about 350 μmol/L to about 400 μmol/L In some embodiments, the subject has a blood phenylalanine level of about 400 μmol/L to about 450 μmol/L. In some embodiments, the subject has a blood phenylalanine level of about 450 μmol/L to about 500 μmol/L. In some embodiments, the subject has a blood phenylalanine level of about 500 μmol/L to about 550 μmol/L. In some embodiments, the subject has a blood phenylalanine level of about 550 μmol/L to about 600 μmol/L. In some embodiments, the subject has a blood phenylalanine level of greater than about 350 μmol/L. In some embodiments, the subject has a blood phenylalanine level of greater than about 360 μmol/L. In some embodiments, the subject has a blood phenylalanine level of greater than about 370 μmol/L. In some embodiments, the subject has a blood phenylalanine level of greater than about 380 μmol/L. In some embodiments, the subject has a blood phenylalanine level of greater than 390 μmol/L. In some embodiments, the subject has a blood phenylalanine level of greater than about 400 μmol/L. In some embodiments, the subject has a blood phenylalanine level of greater than about 600 μmol/L.

In one embodiment, the PKU is classic PKU, e.g., the subject has a tolerance of less than about 20 mg/kg/day of dietary phenylalanine to keep blood concentration of phenylalanine at a safe level, e.g., ≤300 μmol/L. In one embodiment the PKU is moderate PKU, e.g., the subject has a tolerance of about 45-50 mg/kg/day of dietary phenylalanine to keep blood concentration of phenylalanine at a safe level, e.g., ≤300 μmol/L. In one embodiment, the PKU is mild PKU, e.g., the subject has a tolerance of about 55 mg/kg/day of dietary phenylalanine to keep blood concentration of phenylalanine at a safe level, e.g., ≤300 μmol/L. In one embodiment, the HPA is mid-HPA-gray zone, e.g., the subject has a tolerance of about 70 mg/kg/day of dietary phenylalanine to keep blood concentration of phenylalanine at a safe level, e.g., ≤300 μmol/L. In one embodiment, the subject is administered a pharmaceutical composition comprising a plurality of POEGMA-PAL conjugates of Formulas (I), (Ia), (Ia-1), (Ia-1-1), or any of (Ia-1-11)-(Ia-1-46) at a concentration of about 20 mg/mL to about 60 mg/mL to treat PAH deficiency. In one embodiment, the pharmaceutical composition is administered to the subject in an amount of about 0.5 mL to about 1.5 mL. In one embodiment, the pharmaceutical composition is administered to the subject by injection. In one embodiment, the injection is subcutaneous injection. In one embodiment, the pharmaceutical composition is administered to the subject using a pre-filled syringe comprising the pharmaceutical composition. In one embodiment, each subunit of PAL in the POEGMA-PAL conjugate has an amino acid sequence according to SEQ ID NO:4.

Also contemplated herein is use of a plurality of POEGMA-PAL conjugates according to any embodiment in Section 5.1, such as a POEGMA-PAL conjugate of Formulas (I), (Ia), (Ia-1), (Ia-1-1), or any of (Ia-1-11)-(Ia-1-46), a composition according to any embodiment in section 5.3, or pharmaceutical composition according to any embodiment in Section 5.4 in the manufacture of a medicament for the treatment of PAH deficiency in a subject.

Also contemplated herein is a plurality of POEGMA-PAL conjugates according to any embodiment in Section 5.1, such as a POEGMA-PAL conjugate of Formulas (I), (Ia), (Ia-1), (Ia-1-1), or any of (Ia-1-11)-(Ia-1-46), a composition according to any embodiment in section 5.3, or pharmaceutical composition according to any embodiment in Section 5.4 for use in a method of treatment of PAH deficiency in a subject.

In another aspect, the present disclosure provides a method of treating PKU. For example, the POEGMA-PAL conjugates disclosed herein can be used in methods for reducing blood phenylalanine concentration in a subject, wherein the methods comprise administering the POEGMA-PAL conjugates to the patient.

Accordingly, in another aspect, the present disclosure provides a method of treating PKU in a subject, the method comprising administering to the subject pharmaceutical composition, e.g., comprising a composition according to any embodiment in Section 5.3, which comprises a therapeutically effective amount of a plurality of POEGMA-PAL conjugates of Formula (I):

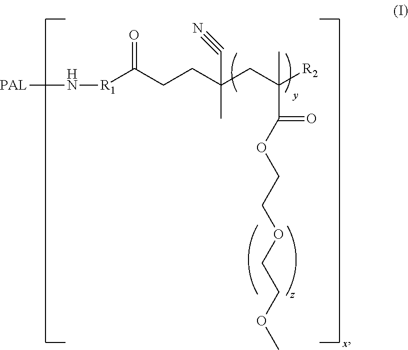

wherein:
PAL is a prokaryotic phenylalanine ammonia-lyase;
R₁ is a linker;
R₂ is an end cap;
x is an integer from 1 to about 32;
y is an integer from about 50 to about 500; and
z is an integer from 2 to about 10.

In one embodiment, the subject has PKU and the administration of the plurality of POEGMA-PAL conjugates to the subject is effective to treat the PKU. In one embodiment, the administration of the POEGMA-PAL is via injection, such as subcutaneous or intramuscular injection. In one embodiment, the pharmaceutical composition comprises a plurality of POEGMA-PAL conjugates at a concentration of about 20 mg/mL to about 60 mg/mL. In one embodiment, the pharmaceutical composition comprises a plurality of POEGMA-PAL conjugates at a concentration of about 20 mg/mL to about 60 mg/mL and is administered to the subject in an amount of about 0.5 mL to about 1.5 mL. In one embodiment, the pharmaceutical composition comprises a plurality of POEGMA-PAL conjugates at a concentration of about 20 mg/mL to about 60 mg/mL and is administered to the subject in an amount of about 0.5 mL to about 1.5 mL using a pre-filled syringe comprising the pharmaceutical composition.

In one embodiment, the POEGMA-PAL conjugates are of Formula (Ia-1-1) as described according to any embodiment in Section 5.1. In one embodiment, x is an integer from about 19 to about 21. In one embodiment, y is an integer from about 175 to about 200. In one embodiment, each subunit of PAL in the POEGMA-PAL conjugate has an amino acid sequence according to SEQ ID NO:4.

In one embodiment, linker R₁ is C(O)—C₁₋₁₂ alkyl-NH. In one embodiment, R₁ is C(O)—(CH₂)₅—NH. Accordingly, in one embodiment, the method of treating PKU in a subject comprises administering to the subject a pharmaceutical composition, e.g., comprising a composition according to any embodiment in Section 5.3, which comprises a therapeutically effective amount of a plurality of POEGMA-PAL conjugates of Formula (Ia):

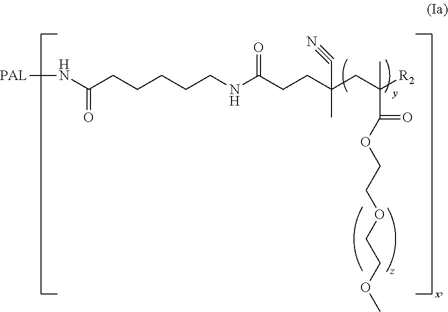

wherein:
PAL is a prokaryotic phenylalanine ammonia-lyase;
R₂ is an end cap;
x is an integer from 1 to about 32;
y is an integer from about 50 to about 500; and
z is an integer from 2 to about 10.

In one embodiment, end cap R₂ is 4-cyanovaleric acid, 4-methoxy-2,4-dimethylvaleronitrile, or hydroxy (—OH). In one embodiment, R₂ is isobutyronitrile. Accordingly, in one embodiment, the method of treating PKU in a subject comprises administering to the subject a pharmaceutical composition, e.g., comprising a composition according to any embodiment in Section 5.3, which comprises a therapeutically effective amount of a plurality of POEGMA-PAL conjugates of Formula (Ia-1):

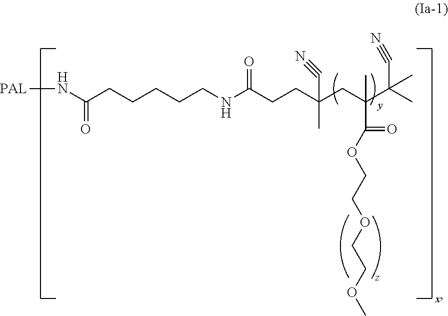

wherein:
PAL is a prokaryotic phenylalanine ammonia-lyase;
x is an integer from 1 to about 32;
y is an integer from about 50 to about 500; and
z is an integer from 2 to about 10.

In one embodiment, z is 2 to about 10. In one embodiment, z is an integer from 2 to about 8, such as 2 to about 5. In certain embodiments, z is 2.

In one embodiment, the method of treating PKU in a subject comprises administering to the subject a pharmaceutical composition, e.g., comprising a composition according to any embodiment in Section 5.3, which comprises a therapeutically effective amount of a plurality of POEGMA-PAL conjugates of Formula (Ia-1-1):

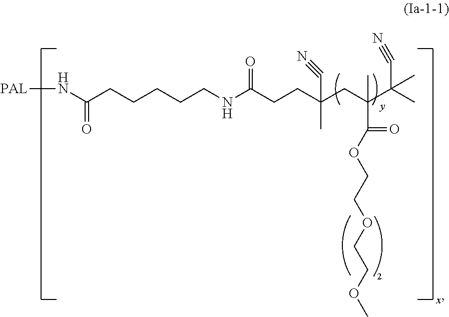

wherein:
PAL is a prokaryotic phenylalanine ammonia-lyase;
x is an integer from 1 to about 32; and
y is an integer from about 50 to about 500.

x and y may be as defined above for Formulas (I), (V), and (VI). In certain embodiments of Formulas (I), (I′), (Ia), (Ia-1), and (Ia-1-1), x may be an integer from about 5 to about 32, about 8 to about 32, about 10 to about 32, about 16 to about 32, about 19 to about 32, about 24 to about 32, about 28 to about 32, about 5 to about 28, about 8 to about 28, about 10 to about 28, about 16 to about 28, about 19 to about 28, about 24 to about 28, about 5 to about 24, about 8 to about 24, about 10 to about 24, about 16 to about 24, about 24 to about 24, about 5 to about 21, about 8 to about 21, about 10 to about 21, about 16 to about 12, or about 19 to about 21. In one embodiment, x is about 19. In one embodiment, x is about 20. In one embodiment, x is about 21. In one embodiment, y is an integer from about 150 to about 300, or about 175 to about 200. Any combination of x and y is encompassed by the present disclosure.

In one embodiment, the method of treating PKU in a subject comprises administering to the subject a pharmaceutical composition, e.g., comprising a composition according to any embodiment in Section 5.3, which comprises a therapeutically effective amount of a plurality of POEGMA-PAL conjugates of one or more of Formulas (Ia-1-11)-(Ia-1-46) shown in Table A.

In one embodiment of using any one of Formulas (I), (I′), (Ia), (Ia-1), and (Ia-1-1) or any one of Formulas (Ia-1-11)-(Ia-1-46) to treat PKU, the subject has a blood phenylalanine level of greater than 360 μmol/L. In some embodiments, the subject has a blood phenylalanine level of about 300 μmol/L to about 350 μmol/L. In some embodiments, the subject has a blood phenylalanine level of about 350 μmol/L to about 400 μmol/L In some embodiments, the subject has a blood phenylalanine level of about 400 μmol/L to about 450 μmol/L. In some embodiments, the subject has a blood phenylalanine level of about 450 μmol/L to about 500 μmol/L. In some embodiments, the subject has a blood phenylalanine level of about 500 μmol/L to about 550 μmol/L. In some embodiments, the subject has a blood phenylalanine level of about 550 μmol/L to about 600 μmol/L. In some embodiments, the subject has a blood phenylalanine level of greater than about 350 μmol/L. In some embodiments, the subject has a blood phenylalanine level of greater than about 370 μmol/L. In some embodiments, the subject has a blood phenylalanine level of greater than about 380 μmol/L. In some embodiments, the subject has a blood phenylalanine level of greater than 390 μmol/L. In some embodiments, the subject has a blood phenylalanine level of greater than about 400 μmol/L. In some embodiments, the subject has a blood phenylalanine level of greater than about 600 μmol/L.

In one embodiment, the PKU is classic PKU, e.g., the subject has a tolerance of less than about 20 mg/kg/day of dietary phenylalanine to keep blood concentration of phenylalanine at a safe level, e.g., ≤300 μmol/L. In one embodiment the PKU is moderate PKU, e.g., the subject has a tolerance of about 45-50 mg/kg/day of dietary phenylalanine to keep blood concentration of phenylalanine at a safe level, e.g., ≤300 μmol/L. In one embodiment, the PKU is mild PKU, e.g., the subject has a tolerance of about 55 mg/kg/day of dietary phenylalanine to keep blood concentration of phenylalanine at a safe level, e.g., ≤300 μmol/L. In one embodiment, the subject is administered a pharmaceutical composition comprising a plurality of POEGMA-PAL conjugates of Formulas (I), (Ia), (Ia-1), (Ia-1-1), or any of (Ia-1-11)-(Ia-1-46) at a concentration of about 20 mg/mL to about 60 mg/mL to treat PKU. In one embodiment, the pharmaceutical composition is administered to the subject in an amount of about 0.5 mL to about 1.5 mL. In one embodiment, the pharmaceutical composition is administered to the subject by injection. In one embodiment, the injection is subcutaneous injection. In one embodiment, the pharmaceutical composition is administered to the subject using a pre-filled syringe comprising the pharmaceutical composition. In one embodiment, each subunit of PAL in the POEGMA-PAL conjugate has an amino acid sequence according to SEQ ID NO:4.

Also contemplated herein is use of a plurality of POEGMA-PAL conjugates according to any embodiment in Section 5.1, such as a POEGMA-PAL conjugate of Formulas (I), (Ia), (Ia-1), (Ia-1-1), or any of (Ia-1-11)-(Ia-1-46), a composition according to any embodiment in section 5.3, or pharmaceutical composition according to any embodiment in Section 5.4 in the manufacture of a medicament for the treatment of PKU in a subject.

Also contemplated herein is a plurality of POEGMA-PAL conjugates according to any embodiment in Section 5.1, such as a POEGMA-PAL conjugate of Formulas (I), (Ia), (Ia-1), (Ia-1-1), or any of (Ia-1-11)-(Ia-1-46), a composition according to any embodiment in section 5.3, or pharmaceutical composition according to any embodiment in Section 5.4 for use in a method of the treatment of PKU in a subject.

In another aspect, the present disclosure provides a method of reducing blood phenylalanine concentration in a subject, the method comprising administering to the subject a pharmaceutical composition according to any embodiment in Section 5.4, which comprises a therapeutically effective amount of a plurality of POEGMA-PAL conjugates of Formula (I):

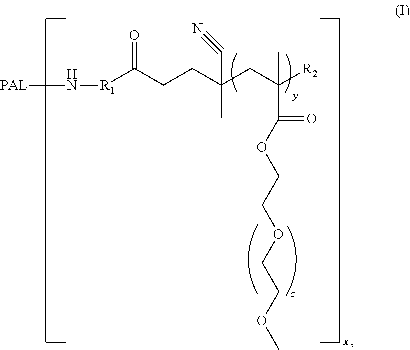

wherein:
PAL is a prokaryotic phenylalanine ammonia-lyase;
R₁ is a linker;
R₂ is an end cap;
x is an integer from 1 to about 21;
y is an integer from about 50 to about 500; and
z is an integer from 2 to about 10.

In another embodiment the method of reducing blood phenylalanine concentration in a subject comprises administering to the subject a therapeutically effective amount of a plurality of POEGMA-PAL conjugates of Formula (I):

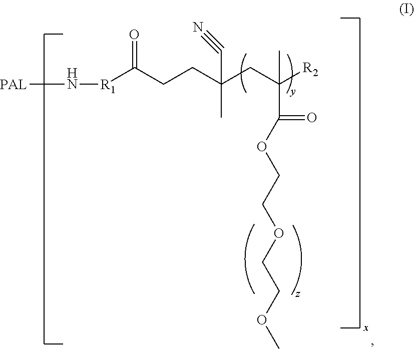

wherein:
PAL is a prokaryotic phenylalanine ammonia-lyase;
R₁ is a linker;
R₂ is an end cap;
x is an integer from 1 to about 32;
y is an integer from about 50 to about 500; and
z is an integer from 2 to about 10.

In one embodiment, the subject has PKU and the administration of the plurality of POEGMA-PAL conjugates to the subject is effective to treat the PKU. In one embodiment, the administration of the POEGMA-PAL is via injection, such as subcutaneous or intramuscular injection. In one embodiment, the pharmaceutical composition comprises a plurality of POEGMA-PAL conjugates at a concentration of about 20 mg/mL to about 60 mg/mL. In one embodiment, the pharmaceutical composition comprises a plurality of POEGMA-PAL conjugates at a concentration of about 20 mg/mL to about 60 mg/mL and is administered to the subject in an amount of about 0.5 mL to about 1.5 mL. In one embodiment, the pharmaceutical composition comprises a plurality of POEGMA-PAL conjugates at a concentration of about 20 mg/mL to about 60 mg/mL and is administered to the subject in an amount of about 0.5 mL to about 1.5 mL using a pre-filled syringe comprising the pharmaceutical composition.

In one embodiment, the POEGMA-PAL conjugates are of Formula (Ia-1-1) as described according to any embodiment in Section 5.1. In one embodiment, x is an integer from about 19 to about 21. In one embodiment, y is an integer from about 175 to about 200. In one embodiment, each subunit of PAL in the POEGMA-PAL conjugate has an amino acid sequence according to SEQ ID NO:4.

In one embodiment, linker R₁ is C(O)—C₁₋₁₂ alkyl-NH. In one embodiment, R₁ is C(O)—(CH₂)₅—NH. Accordingly, in one embodiment, the method of reducing blood phenylalanine concentration in a subject (e.g., to treat PKU) comprises administering to the subject a pharmaceutical composition, as described in any embodiment in Section 5.4, comprising a therapeutically effective amount of a plurality of POEGMA-PAL conjugates of Formula (Ia):

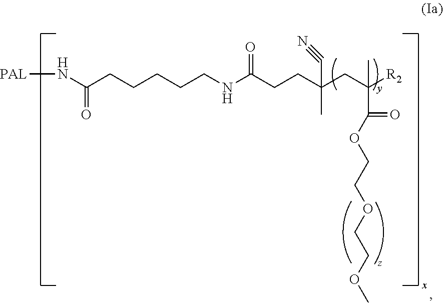

wherein:
PAL is a prokaryotic phenylalanine ammonia-lyase;
R₂ is an end cap;
x is an integer from 1 to about 21;
y is an integer from about 50 to about 500; and
z is an integer from 2 to about 10.

In another embodiment, the method of reducing blood phenylalanine concentration in a subject (e.g., to treat PKU) comprises administering to the subject a pharmaceutical composition comprising a therapeutically effective amount of a plurality of POEGMA-PAL conjugates of Formula (Ia):

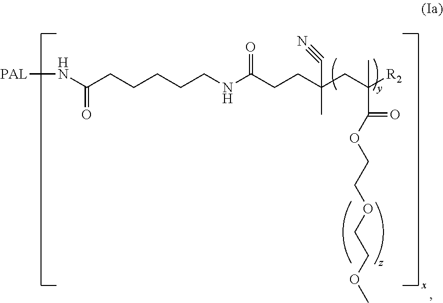

wherein:
PAL is a prokaryotic phenylalanine ammonia-lyase;
R₂ is an end cap;
x is an integer from 1 to about 32;
y is an integer from about 50 to about 500; and
z is an integer from 2 to about 10.

In one embodiment, end cap R₂ is 4-cyanovaleric acid, 4-methoxy-2,4-dimethylvaleronitrile, or hydroxy (—OH). In one embodiment, R₂ is isobutyronitrile. Accordingly, in one embodiment, the method of reducing blood phenylalanine concentration in a subject (e.g., to treat PKU) comprises administering to the subject a pharmaceutical composition, e.g., comprising a composition according to any embodiment in Section 5.3, comprising a therapeutically effective amount of a plurality of POEGMA-PAL conjugates of Formula (Ia-1):

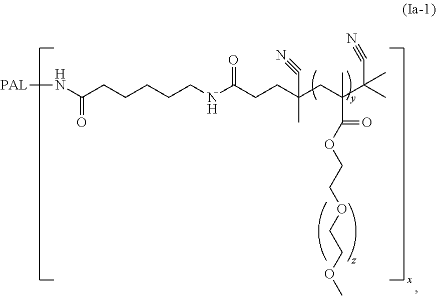

wherein:
PAL is a prokaryotic phenylalanine ammonia-lyase;
x is an integer from 1 to about 21;
y is an integer from about 50 to about 500; and
z is an integer from 2 to about 10.

In another embodiment, the method of reducing blood phenylalanine concentration in a subject (e.g., to treat PKU) comprises administering to the subject a pharmaceutical composition comprising a therapeutically effective amount of a plurality of POEGMA-PAL conjugates of Formula (Ia-1):

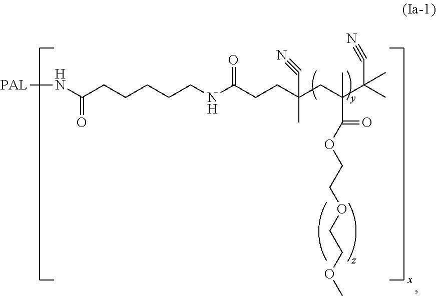

wherein:
PAL is a prokaryotic phenylalanine ammonia-lyase;
x is an integer from 1 to about 32;
y is an integer from about 50 to about 500; and
z is an integer from 2 to about 10.

In one embodiment, z is 2 to about 10. In one embodiment, z is an integer from 2 to about 8, such as 2 to about 5. In certain embodiments, z is 2.

In one embodiment, the method of reducing blood phenylalanine concentration in a subject (e.g., to treat PKU) comprises administering to the subject a pharmaceutical composition, e.g., comprising a composition according to any embodiment in Section 5.3, comprising a therapeutically effective amount of a plurality of POEGMA-PAL conjugates of Formula (Ia-1-1):

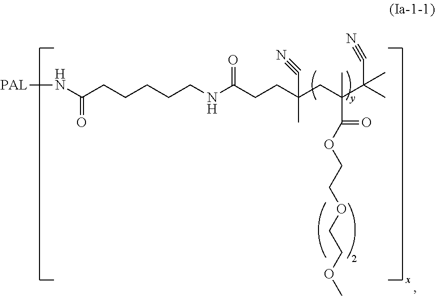

wherein:
PAL is a prokaryotic phenylalanine ammonia-lyase;
x is an integer from 1 to about 21; and
y is an integer from about 50 to about 500.

In another embodiment, the method of reducing blood phenylalanine concentration in a subject (e.g., to treat PKU) comprises administering to the subject a pharmaceutical composition comprising a therapeutically effective amount of a plurality of POEGMA-PAL conjugates of Formula (Ia-1-1):

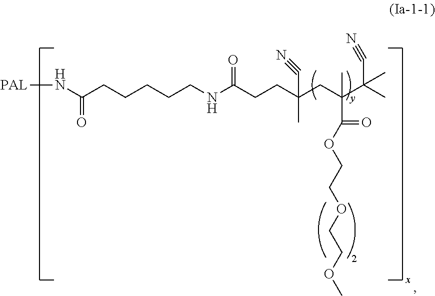

wherein:
PAL is a prokaryotic phenylalanine ammonia-lyase;
x is an integer from 1 to about 32; and
y is an integer from about 50 to about 500.

x and y may be as defined above for Formulas (I), (V), and (VI). In certain embodiments of Formulas (I), (I′), (Ia), (Ia-1), and (Ia-1-1), x may be an integer from about 5 to about 21, 8 to about 21, about 10 to about 21, about 16 to about 21, or about 19 to about 21. In one embodiment, y is an integer from about 150 to about 300, or about 175 to about 200. Any combination of x and y is encompassed by the present disclosure.

In certain embodiments, x is an integer from about 5 to about 32, about 8 to about 32, about 10 to about 32, about 16 to about 32, about 19 to about 32, about 24 to about 32, about 28 to about 32, about 5 to about 28, about 8 to about 28, about 10 to about 28, about 16 to about 28, about 19 to about 28, about 24 to about 28, about 5 to about 24, about 8 to about 24, about 10 to about 24, about 16 to about 24, about 19 to about 24, about 5 to about 21, about 8 to about 21, about 10 to about 21, about 16 to about 12, or about 19 to about 21. In one embodiment, x is about 19. In one embodiment, x is about 20. In one embodiment, x is about 21. In one embodiment, y is an integer from about 150 to about 300, or about 175 to about 200.

In one embodiment, the method of reducing blood phenylalanine concentration in a subject comprises administering to the subject a pharmaceutical composition, e.g., comprising a composition according to any embodiment in Section 5.3, which comprises a therapeutically effective amount of a plurality of POEGMA-PAL conjugates of one or more of Formulas (Ia-1-11)-(Ia-1-46) shown in Table A.

In one embodiment of using any one of Formulas (I), (I′), (Ia), (Ia-1), and (Ia-1-1), or any one of Formulas (Ia-1-11)-(Ia-1-46) to reduce blood phenylalanine concentrations, the subject has a blood phenylalanine level of greater about 300 μmol/L to about 350 μmol/L. In some embodiments, the subject has a blood phenylalanine level of about 350 μmol/L to about 400 μmol/L In some embodiments, the subject has a blood phenylalanine level of about 400 μmol/L to about 450 μmol/L. In some embodiments, the subject has a blood phenylalanine level of about 450 μmol/L to about 500 μmol/L. In some embodiments, the subject has a blood phenylalanine level of about 500 μmol/L to about 550 μmol/L. In some embodiments, the subject has a blood phenylalanine level of about 550 μmol/L to about 600 μmol/L. In some embodiments, the subject has a blood phenylalanine level of greater than about 350 μmol/L. In some embodiments, the subject has a blood phenylalanine level of greater than about 360 μmol/L. In some embodiments, the subject has a blood phenylalanine level of greater than about 370 μmol/L. In some embodiments, the subject has a blood phenylalanine level of greater than about 380 μmol/L. In some embodiments, the subject has a blood phenylalanine level of greater than 390 μmol/L. In some embodiments, the subject has a blood phenylalanine level of greater than about 400 μmol/L. In some embodiments, the subject has a blood phenylalanine level of greater than about 600 μmol/L.

In one embodiment, the subject has PKU. In one embodiment, the PKU is classic PKU, e.g., the subject has a tolerance of less than about 20 mg/kg/day of dietary phenylalanine to keep blood concentration of phenylalanine at a safe level, e.g., ≤300 μmol/L. In one embodiment the PKU is moderate PKU, e.g., the subject has a tolerance of about 45-50 mg/kg/day of dietary phenylalanine to keep blood concentration of phenylalanine at a safe level, e.g., ≤300 μmol/L. In one embodiment, the PKU is mild PKU, e.g., the subject has a tolerance of about 55 mg/kg/day of dietary phenylalanine to keep blood concentration of phenylalanine at a safe level, e.g., ≤300 μmol/L.

In one embodiment the subject has HPA. In one embodiment, the HPA is mid-HPA-gray zone, e.g., the subject has a tolerance of about 70 mg/kg/day of dietary phenylalanine to keep blood concentration of phenylalanine at a safe level, e.g., ≤300 μmol/L.

Also contemplated herein is use of a plurality of POEGMA-PAL conjugates according to any embodiment in Section 5.1, such as a POEGMA-PAL conjugate of Formulas (I), (Ia), (Ia-1), (Ia-1-1), or any of (Ia-1-11)-(Ia-1-46), a composition according to any embodiment in section 5.3 or pharmaceutical composition according to any embodiment in Section 5.4 in the manufacture of a medicament for reducing phenylalanine plasma levels in a subject.

Also contemplated herein is a plurality of POEGMA-PAL conjugates according to any embodiment in Section 5.1, such as a POEGMA-PAL conjugate of Formulas (I), (Ia), (Ia-1), (Ia-1-1), or any of (Ia-1-11)-(Ia-1-46), a composition according to any embodiment in section 5.3 or pharmaceutical composition according to any embodiment in Section 5.4 for use in a method of reducing phenylalanine plasma levels in a subject.

In one embodiment of Formula (I), (I′), (Ia), (Ia-1), (Ia-1-1), or any of (Ia-1-11)-(Ia-1-46), PAL is a wild-type PAL from a prokaryotic organism, including, but not limited to, PAL from Streptomyces maritimus, Nostoc punctiforme, Anabaena variabilis, Anacystis nidulans, Photorabdus luminescens TT01, and Streptomyces verticillatus. In one embodiment, PAL is a fragment, mutant, variant, or analog of a wild-type PAL enzyme that retains similar (e.g., at least 50%) or increased catalytic activity for phenylalanine. In one embodiment, PAL is a prokaryotic PAL from Anabaena variabilis (SEQ ID NO:1) or biologically active fragment, mutant, variant or analog thereof. In certain embodiments, PAL is an Anabaena variabilis PAL (AvPAL) variant. In one embodiment, PAL is an AvPAL variant wherein one or more amino acid residues have been substituted by another amino acid residue. In some embodiments, one or more cysteine residues of the AvPAL variant have been substituted by a serine residue. In some embodiments, the one or more cysteine residues of the AvPAL variant that have been substituted by one or more serine residues are selected from the group consisting of cysteine residues at positions 503 and 565 in each subunit. In one embodiment, each subunit of PAL has an amino acid sequence according to SEQ ID NO: 2. In one embodiment, each subunit of PAL has an amino acid sequence according to SEQ ID NO:3. In one embodiment, each subunit of PAL has an amino acid sequence according to SEQ ID NO:4.

The dosing frequency of the POEGMA-PAL conjugates to the subject will vary depending upon the condition being treated. In certain embodiments, the POEGMA-PAL conjugates (or pharmaceutical composition comprising the same) may be administered to a subject about two times per week, about one time per week, about one time every two weeks, about one time per month, or less frequently than about one time per month. It is understood that, in any subject, the dosing frequency determined to be most therapeutic may vary from a frequency disclosed herein due to variations in responses by different individuals to the POEGMA-PAL conjugates. The term “about” is intended to reflect such variations.

In certain embodiments, the POEGMA-PAL conjugates may be administered to a subject less frequently than native PAL while still providing equal or greater therapeutic benefit to the subject. In certain embodiments, the POEGMA-PAL conjugates may be administered to a subject less frequently than pegylated PAL while still providing equal or greater therapeutic benefit to the subject. In certain embodiments, the POEGMA-PAL conjugates may be administered to a subject less frequently than Pegvaliase while still providing equal or greater therapeutic benefit to the subject.

In certain embodiments, a method of reducing blood phenylalanine concentration in a subject (e.g., to treat PKU or PAH deficiency) may further comprise monitoring the blood phenylalanine levels of the subject, e.g., on a regular basis such as every 2 weeks, every 4 week, every 6 weeks, every 8 weeks, or every 10 weeks.

In another embodiment of the methods provided herein, the subject is administered a low or modified protein diet, or a low or modified phenylalanine diet in combination with a pharmaceutical composition disclosed herein, such that plasma phenylalanine levels are decreased, e.g., by at least about 25%. See, e.g., U.S. Pat. Nos. 7,531,341 and 7,534,595 for further information on the management of patient populations with elevated phenylalanine levels (e.g., HPA and PKU), which, in certain embodiments, can be used in conjunction with the methods provided herein. Each of U.S. Pat. Nos. 7,531,341 and 7,534,595 are incorporated herein by reference in its entirety.

### 6. EXAMPLES

### Example 1: POEGMA Polymer Synthesis

### First Exemplary 45 kDa POEGMA-C₆—NHS Synthesis

45 kDa POEGMA-C6-NHS was synthesized using reversible addition-fragmentation chain-transfer (RAFT) polymerization. All reagents were purchased from Sigma-Aldrich with the exception of the chain transfer agent (CTA): ECT-ahx-NHS (2,5-dioxopyrrolidin-1-yl 6-(4-cyano-4-(((ethylthio) carbonothioyl)thio) pentanamido) hexanoate) which was produced by Sai. Tri-ethylene glycol methyl ether methacrylate (4.31 mmol), the CTA (19.6 μmol), and anhydrous DMSO (4 mL) were mixed in a polymerization flask (5 mL RBF). The polymerization flask was sealed with a septum and purged with nitrogen to remove oxygen. The flask was then transferred to a stir plate equipped with a blue LED light source. The flask and the light source were encased in tinfoil and the flask was exposed to high intensity blue LED light for 2.5 hours. The resulting polymer was purified via diethyl ether precipitation and dried under vacuum to yield POEGMA-C₆—NHS

### 45 kDa POEGMA-C₆—NHS End-Group-Removal (EGR):

POEGMA-C6-NHS (22.2 μmol) and azobisisobutyronitrile (2.2 mmol) were dissolved in anhydrous DMSO (5 mL) and transferred into a reaction flask. The reaction flask was sealed with a septum and purged with nitrogen to remove oxygen. The purged vessel was transferred to a 60° C. oil bath and left to react for 2 hours. After 2 hours the reaction solution was exposed to oxygen to quench and was purified via diethyl ether precipitation and dried under vacuum to yield POEGMA-C₆—NHS without the trithiocarbonate from the RAFT chain transfer agent used in polymerization.

### Second Exemplary 45 kDa POEGMA-C₆—NHS Synthesis

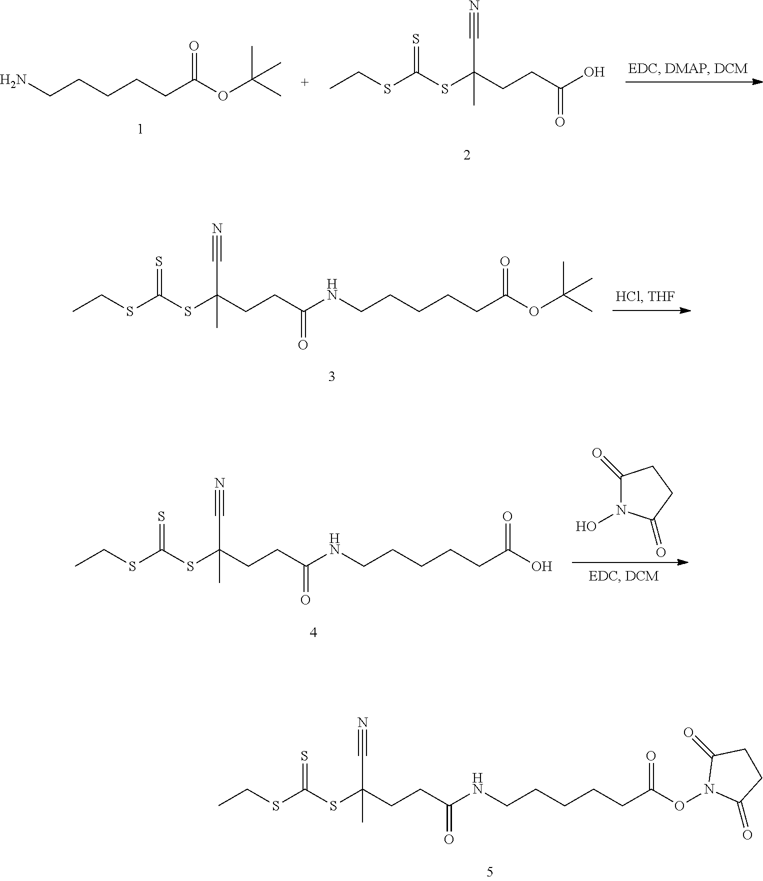

The synthesis of compound (5) begins with an amide coupling using 1-ethyl-3-(3-dimethylaminopropyl) carbodiimide (EDC) and 4-dimehtylaminopyridine (DMAP) to couple tert-butyl 6-aminohexanoate (1) and the chain transfer agent 4-cyano-4-(((ethylthio) carbonothioyl)thio) pentanoic acid (2), to form compound 3, which is then Boc deprotected in the presence of acid to yield compound 4. The last step of the synthesis is the NHS coupling to form 2,5-dioxopyrrolidin-1-yl 6-(4-cyano-4 (((ethylthio) carbonothioyl)thio) pentanamido) hexanoate (5). NHS-POEGMA is then obtained in two steps starting from compound 5 and 6, as shown below:

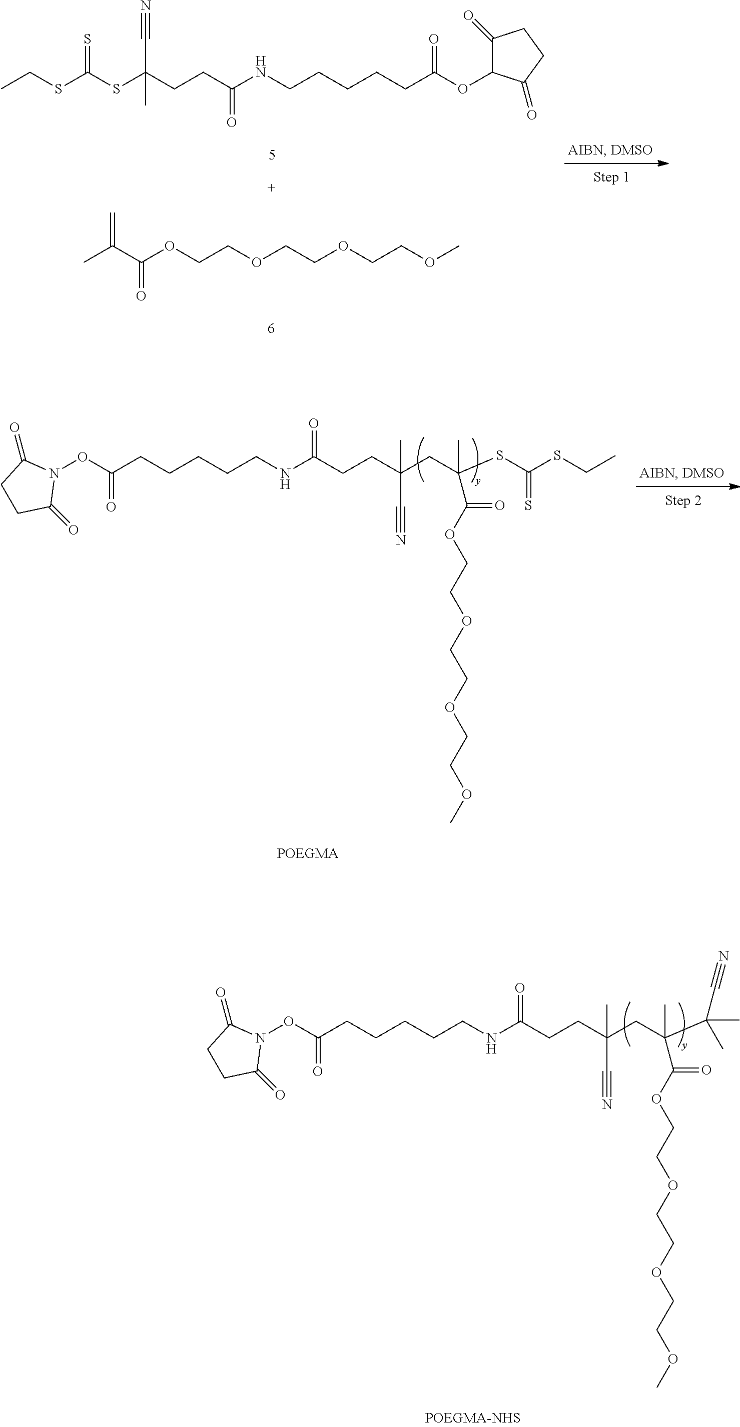

Step 1 involves the polymerization reaction of compound (5) and compound (6) in the presence of the radical initiator AIBN, under an inert atmosphere. Once the reaction is completed, POEGMA is precipitated using MTBE and filtered. The resultant crude product is further purified by re-precipitation, by first dissolving it in DMSO and then, precipitating using MTBE. In the Step 2, the trithiocarbonate end group is removed, using AIBN in DMSO, under an inert atmosphere. Upon reaction completion, POEGMA-NHS is precipitated using MTBE, filtered, and purified using re-precipitation conditions defined in Step 1.

### Example 2: POEGMA-PAL Bioconjugation and Purification

POEGMA-C6-NHS was weighed out in a flask and solvated in cold, sterile diH₂O at ˜450 mg/mL. An appropriate amount of PAL protein in ‘ready-to-conj’ PAL buffer was aliquoted to a vessel as well as a volume of the ‘ready-to-conj’ buffer that is equivalent to the polymer solution added. The polymer solution was then added to the PAL protein diluted in the ‘ready-to-conj’ PAL buffer. A 4-fold molar excess to surface exposed lysines on a subunit of PAL of polymer was added. The reaction was left on an orbital shaker at room temperature for 1 hour to allow the NHS of the POEGMA-C₆—NHS polymer to react with surface exposed lysines and transferred to 5° C. storage overnight. The final conjugate solution was purified via tangential flow filtration (TFF) with a 100 kDa molecular weight cutoff Hydrostart membrane. During this process, the buffer composition was replaced with a buffer consisting of 19.98 mM Tris and 136 mM NaCl, at a pH of ˜7.4 (1×TBS).

### Example 3: Viscosity Assessment

Viscosity of the purified POEGMA-PAL conjugate was assessed via an m-VROC viscometer. The conjugates were tested at a concentration of 20±2 mg/mL in 1×TBS in a 500 μL syringe. POEGMA-C₆—NHS solvated in 1×TBS was also tested at various concentrations in a 500 μL syringe. The sheer force applied to the syringes was adjusted for each sample so the viscometer was operated at ˜80-90% of its threshold for pressure limits.

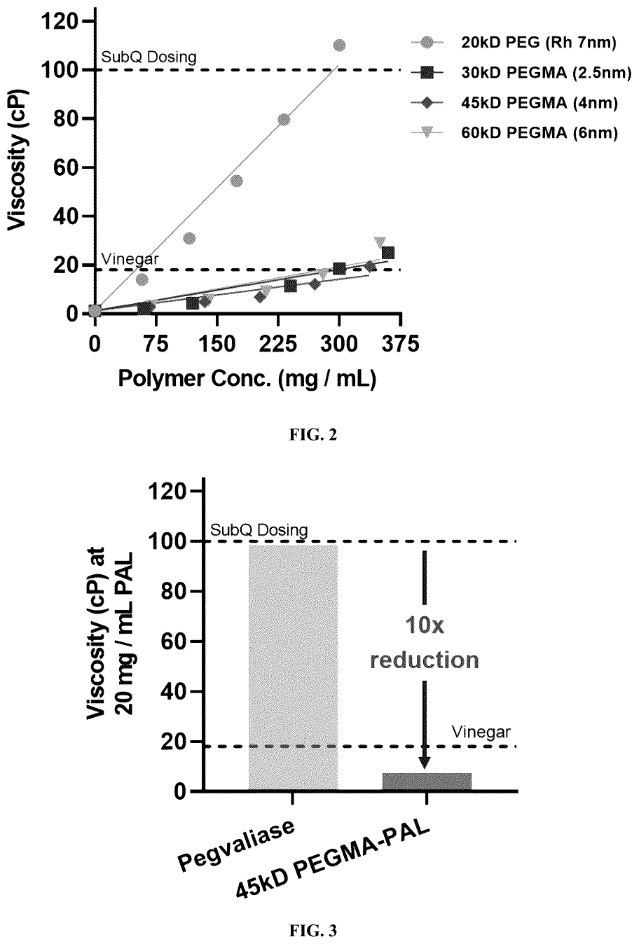

FIG. 2 reports the viscosity of 20 kDa PEG-PAL (Rₕ=7 nm), 30 kDa POEGMA-PAL (Rₕ=2.5 nm), 45 kDa POEGMA-PAL (Rₕ=4 nm), and 60 kDa POEGMA-PAL (Rₕ=6 nm) at increasing concentrations. While the viscosity of the PEG-PAL solution quickly increases with increasing concentrations, the viscosity of the POEGMA-PAL solution increases at a much lower rate and, even at concentrations greater than 300 mg/mL PAL, has a consistency like that of vinegar (˜ 20 cP). Similarly, FIG. 3 reports the viscosity of two solutions having equivalent concentrations of PAL (20 mg/mL) but conjugated to a different polymer (Pegvaliase versus POEGMA-PAL). Notably, the Pegvaliase solution has a viscosity of about 100 cP whereas the POEGMA-PAL solution has a much lower viscosity of less than about 10 cP. As one of skill in the art will recognize, there is limited volume of liquid that can be administered to a subject via subcutaneous injection, however delivering a large dose of a therapeutic agent in a small volume can often prove problematic due to the high viscosity of the solution, as highly viscous fluids do not flow easily through a syringe and needle and can cause pain at the injection site. However, by formulating PEG as a conjugate with POEGMA, an injectable solution can contain a high dose of PAL without exhibiting high viscosity which is easily administrable via subcutaneous injection.

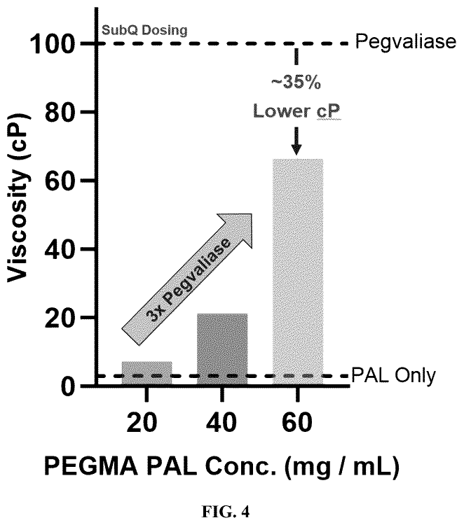

FIG. 4 reports the viscosity of a 20 mg/mL, 40 mg/mL, and 60 mg/mL 45 kDa POEGMA-PAL solution compared to the viscosity of the clinical dose of Pegvaliase (100 cP). The 60 mg/mL POEGMA-PAL solution has a viscosity that is 35% lower than the 20 mg/mL PEG-PAL solution used to deliver the highest clinical dose of Pegvaliase. Accordingly, using the POEGMA-PAL conjugates disclosed herein will allow access to a three-fold higher concentration of POEGMA-PAL per dose, thereby reducing the daily number of required injections from three to one (or less).

### Example 4: Anti-Polymer IgG Assessment

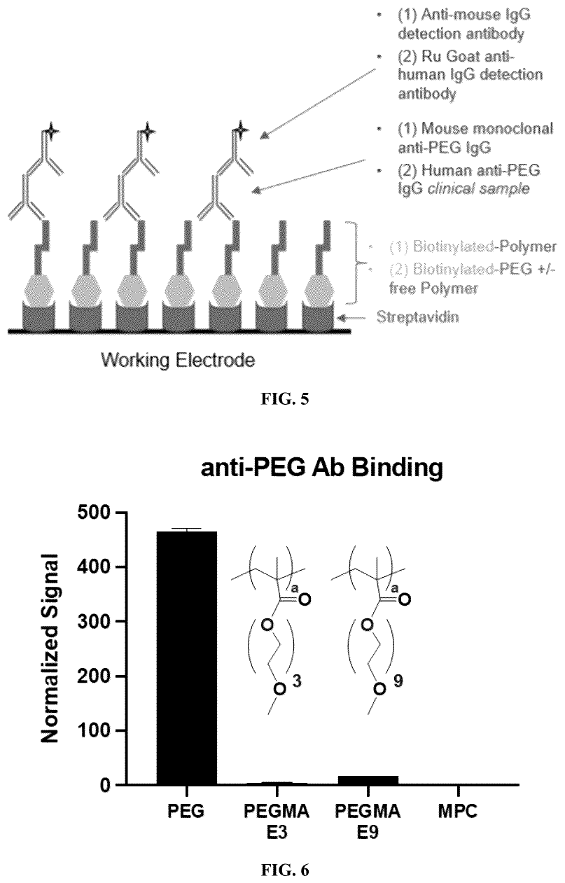

Meso Scale Discovery® platform was used to assess the anti-polymer IgG in mice and human serum samples. A scheme of the experiments undertaken is shown in FIG. 5. MSD GOLD Streptavidin plates were blocked with 150 μL of casein in PBS blocking buffer at room temperature for 45±15 minutes with shaking. Biotinylated-POEGMA coating solution was prepared in 1×DPBS at a final concentration of 4 μg/mL. Linear PEG (20 kDa) coating solution was prepared in 1×DPBS at a final concentration of 0.25 μg/mL MPC solution was prepared in 1×DPBS at a final concentration of 4 μg/mL. After removing the blocking buffer, 50 μL of either the biotinylated-POEGMA coating solution or linear PEG coating solution was applied to the MSD wells for 60±10 minutes at room temperature with shaking. The MSD plate was washed using a plate washer 3 times to remove unbound polymer. Serum samples were prepared at 1:10 MRD in casein in PBS. 50 μL of prepared serum samples were applied to the MSD wells for 60±10 minutes at room temperature with shaking. MSD plate was washed using a plate washer 3 times. Sulfo-tagged AffiniPure F(ab′)₂ Fragment Goat Anti-Mouse IgG (detection antibody for mouse assay) and AffiniPure F(ab′)₂ Fragment Goat Anti-Human IgG (detection antibody for human assay) were prepared at final concentration of 0.1 μg/mL and 0.5 μg/mL in casein in PBS, respectively. 50 μL of detection antibody were applied to the MSD wells for 60±10 minutes at room temperature with shaking while protected from light. MSD plate was washed using a plate washer 3 times. 1×MSD read buffer was prepared from 4×MSD read buffer T with surfactants. 150 μL of 1×MSD read buffer was added to the MSD wells and the plate was read using MESO QuickPlex SQ within 10 minutes of adding the buffer.

FIG. 6 reports the cross-reactivity of mouse monoclonal anti-PEG IgG with each of a linear PEG, MPC, and two different POEGMA polymers (E3 and E9). While PEG exhibits high immunogenicity, the anti-PEG IgG does not appear to bind to the POEGMA polymers strongly.

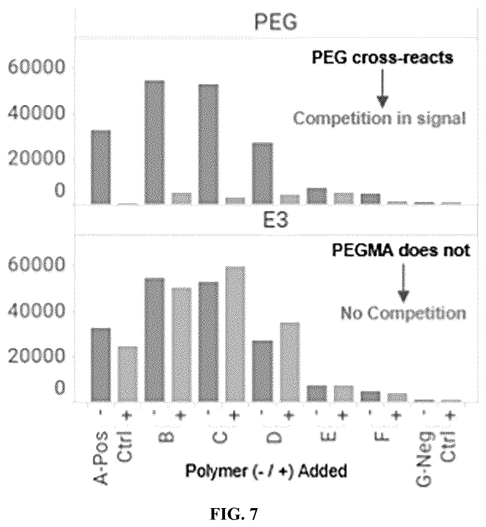

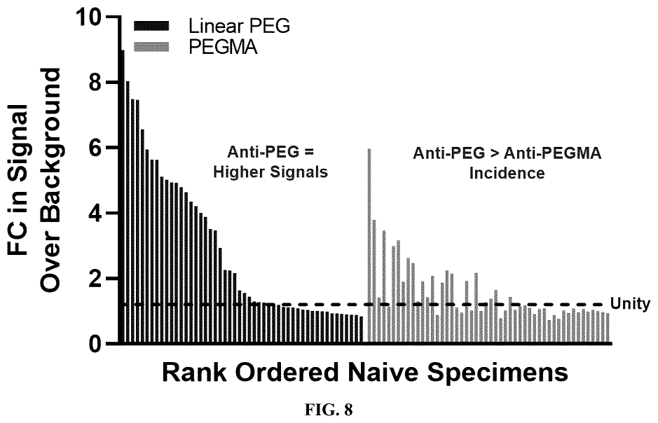

To test the antigenicity of POEGMA in naïve subjects and subjects treated with Pegvaliase, a Meso Scale Discovery® platform was used to assess the pre-existing anti-PEG and anti-POEGMA IgG in naive human serum samples. MSD GOLD Streptavidin plates were blocked with 150 μL of casein in PBS blocking buffer at room temperature for 45±15 minutes with shaking. Biotinylated-POEGMA (45 kDa) coating solution was prepared in 1×DPBS at the final concentration of 4 μg/mL. Biotinylated-PEG (20 kDa) coating solution was prepared in 1×DPBS at a final concentration of 0.25 μg/mL. After removing the blocking buffer, 50 μL of the respective biotinylated-polymer coating solution was applied to the MSD wells for 60±10 minutes at room temperature with shaking. The MSD plate was washed 3 times via aspiration to remove unbound polymer. Serum samples were prepared at 1:10 MRD in casein in PBS. 50 μl of prepared serum samples were applied to the MSD wells for 60±10 minutes at room temperature with shaking. MSD plate was then washed 3 times via aspiration. AffiniPure F(ab′)₂ Fragment Goat Anti-Human IgG was diluted to 0.5 μg/mL in casein in PBS. 50 μL of detection antibody was applied to the MSD wells for 60=10 minutes at room temperature with shaking while protected from light. MSD plate was washed via aspiration 3 times. 1×MSD read buffer was prepared from 4×MSD read buffer T with surfactants. 150 μL of 1×MSD read buffer was added to the MSD wells and the plate was read using MESO QuickPlex SQ within 10 minutes of adding the buffer. FIG. 7 reports the cross-reactivity of PEG (top) and 45 kDa POEGMA polymer (bottom) and in anti-PEG IgG from Pegvaliase-treated subjects with existing anti-PEG IgG from Pegvaliase-treated subjects. In contrast to PEG, the POEGMA polymer does not cross-react with anti-PEG IgG present in the plasma of Pegvaliase-treated subjects. FIG. 8 reports the antigenicity of linear PEG compared to a 45 kDa POEGMA polymer, as disclosed herein, in naïve subjects, demonstrating that both the titer levels and incidence rates for pre-existing anti-POEGMA antigenicity are lower than that for anti-PEG.

### Example 5: Animal Studies

45 kDa POEGMA-PAL (5 mL/kg), 10 kDa linear PEG-PAL (5 ml/kg), Pegvaliase (5 mL/kg), or vehicle (1×TBS; 5 mL/kg) were administered to male PAH^{enu2-/-} mice (n=5) by subcutaneous (SC) injection on every Monday, Wednesday, and Friday for 8 weeks. Vehicle was also administered to wild-type mice according to the same timeline. The dose level of POEGMA-PAL was 10 mg/kg on Monday and Wednesday and 20 mg/kg on Friday. The weight of each mouse was measured and recorded prior to each administration.

Blood was sampled from each animal every Tuesday and Thursday during POEGMA-PAL administration period time by making a transverse nick in the skin of the tail, approximately 0.1 cm in length, using a sterile surgical blade, after warming the mouse briefly under a heating lamp to induce vasodilation. The ensuing blood was collected into a single lithium-heparinized capillary tube (Microvette® CB 300 tube, Sarstedt Inc, #16.443.100), which was immediately centrifuged to yield plasma. The plasma was decanted into clean, labeled tubes and stored at −80° C.

At the terminal timepoint (9 weeks post-dose), each animal was deeply anesthetized with isoflurane delivered via nose cone (3% for induction, 1.5% for maintenance). A terminal blood sample was collected via cardiac puncture and placed in a tube containing lithium heparin (BD Biosciences, #365965). Blood samples were centrifuged to yield plasma, which was decanted into clean, labeled tubes and stored at −80° C. The animals were then euthanized by exsanguination, performance of a thoracotomy. All biological samples were transferred to the analytical lab on dry ice.

Plasma phenylalanine concentrations were determined via liquid-liquid extraction, followed by hydrophilic interaction chromatography separation and multiple reaction monitoring mass spectrometry (MS) Analysis. Standard curves and QCs were generated via serial dilution of reference phenylalanine standard in 5% BSA in NaCl. Calibration range was set from 4 μg/mL to 1000 μg/mL with quality control standards at 15 μg/mL and 200 μg/mL. 10 μL aliquots of plasma, standards, and QCs were standardized with heavy isotope labelled phenylalanine, and extracted with 300 μL Acetonitrile. Supernatants were diluted 1:5 in 0.1% formic acid 2 mM Ammonium Acetate 85:5:10 acetonitrile:methanol:water. Extracts were injected and separated on an Atlantis Premier BEH Z-HILIC 1.7 μm; 2.1×50 mm column at 55° C. and separated with an elution gradient. Phenylalanine was quantified on the transition 166.1->120.1 m/z (172.1->126 m/z for IS). Normalized peak area counts were used to calculate concentration using the generated regression curve. Accuracy criteria for QC and calibrators was +/−20% of nominal value.

Plasma PAL concentrations were determined via in-solution trypsin digestion, followed by targeted peptide quantitation using reverse phase liquid chromatography and multiple reaction monitoring MS analysis. Quality control and calibration samples generated by serial dilution of PAL in mouse plasma. Calibration range was set from 50 μM to 5000 μM with quality control standards at 150 μM, 1500 μM, and 3750 μM. 2 μL of each sample, standard, or QC were spiked with heavy isotope labelled peptide standard. Standardized samples were then reduced and alkylated with tris (2-carboxyethyl) phosphine (TCEP) and chloroacetamide respectively, followed by in-solution trypsin digestion overnight. Digested peptide extracts were purified and concentrated using Sepak C18 SPE plates. Final extracts were injected onto Acquity Peptide BEH C18; 1.7 μm; 2.1×150 mm column at 55° C. and separated using an elution gradient. Surrogate PAL peptide and corresponding heavy labelled IS peptide were quantified on the following transitions: PAL 466.3->573.3 m/z; PAL IS 471.3->583.3 m/z. Normalized peak area counts were used to calculate concentration using the generated regression curve. Accuracy criteria for QC and calibrators was +/−20% of nominal value.

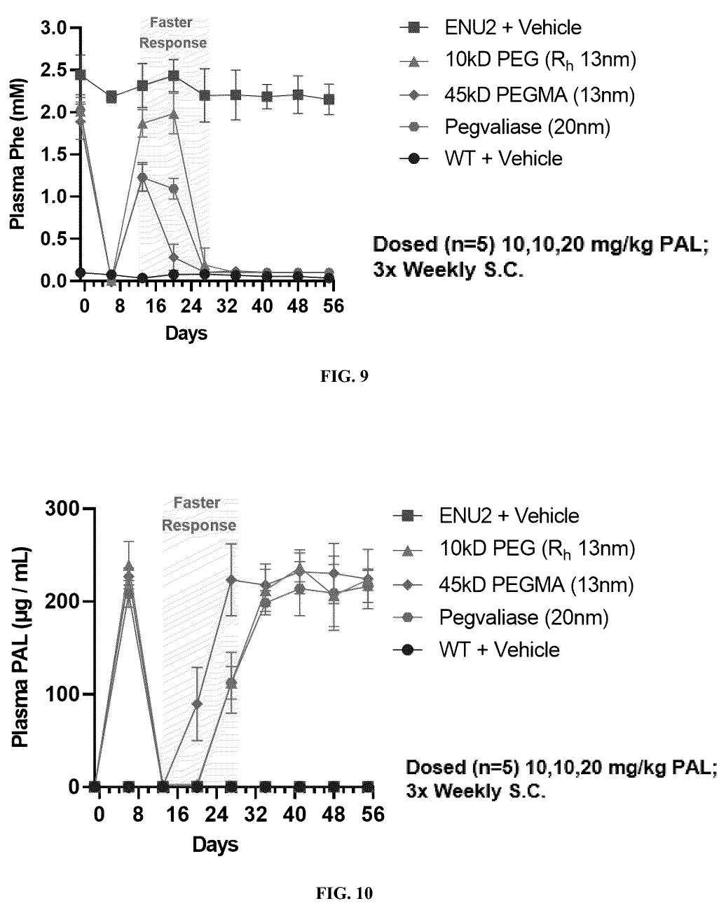

FIGS. 9 and 10 report plasma phenylalanine (Phe) and plasma PAL levels, respectively, at each timepoint plasma was collected in each of the mice. Pegvaliase (Rₕ=20 nm) sustains lower phenylalanine levels in the mice in comparison to linear 10 kDa PEG-PAL (Rₕ=13 nm), suggesting that maximizing the size of the PAL conjugate improves efficacy. However, similarly sized POEGMA-PAL (Rₕ=13-14 nm) performs better than Pegvaliase, demonstrating pharmacokinetics of POEGMA-PAL are twice as fast.

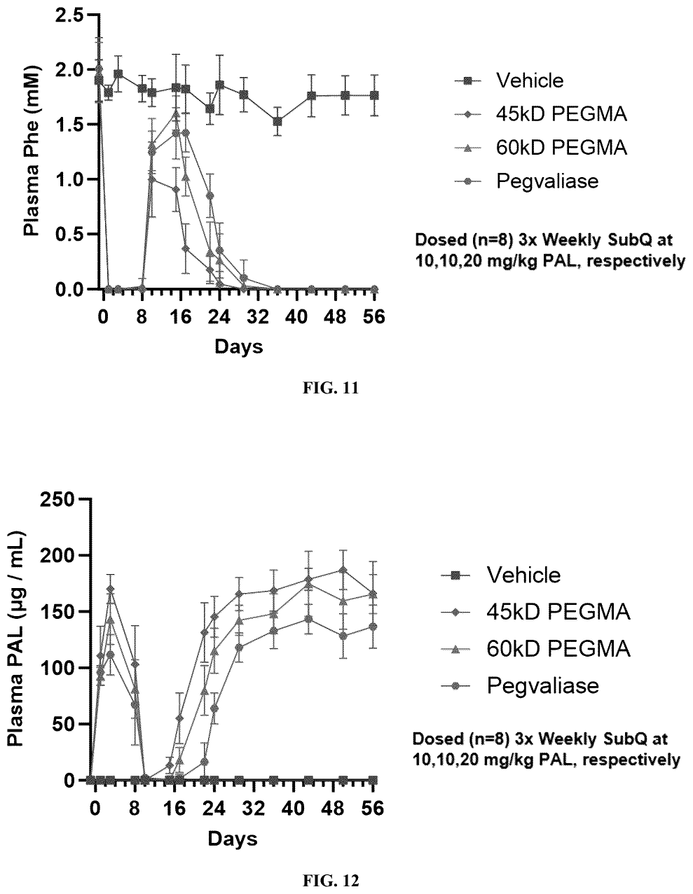

The same experiment was undertaken to understand the efficacy of 45 kDa POEGMA-PAL versus a larger 60 kDa POEGMA-PAL. 45 kDa POEGMA-PAL (10 mL/kg), 60 kDa POEGMA-PAL (10 mL/kg), Pegvaliase (20 mL/kg), or vehicle (1×TBS; 5 mL/kg) were administered to male PAH^{enu2-/-} mice (n=8) by subcutaneous (SC) injection three times weekly. Vehicle was also administered to wild-type mice according to the same timeline. The weight of each mouse was measured and recorded prior to POEGMA-PAL or vehicle administration. Blood was sampled, stored, and analyzed as described above. FIGS. 11 and 12 depict plasma phenylalanine (Phe) and plasma PAL levels, respectively, at each timepoint plasma was collected in each of the mice.

Pegvaliase demonstrated a loss of exposure and efficacy after 1 week treatment followed by a gradual return to response over two weeks. With the same treatment regimen, 45k POEGMA-PAL is faster to respond: the time to phenylalanine reduction (long-term pharmacodynamic effect) after an initial loss of efficacy is reduced by 50%.

### Example 6: Peptide Mapping

Subtractive peptide mapping was leveraged to understand differences in lysine site occupancy between Pegvaliase and 45k POEGMA-PAL upon polymer conjugation. Aliquots of 45 kDa POEGMA-PAL, Pegvaliase, and un-conjugated rAvPAL are diluted by 4× with 50 mM Tris buffer. Bio-Rad P6 gel filtration spin columns were used for removal of small molecular weight contaminants for un-conjugated rAvPAL. Total protein measurements were made using nanodrop A280 for each aliquot. A constant quantity of protein was denatured via addition of Urea, and incubation at room temperature for 1 hour. Denatured samples were diluted 10× with 50 mM Tris and digested using trypsin/LysC for 20 hours at 37° C. Digested samples were reduced with DTT and acidified with addition of 15% formic acid in water.

Digested samples were analyzed by LC-MS using DDA top15 acquisition. 10 μL injections were made on Acquity UPLC Peptide BEH C18 column, 300 A, 1.7 μm, 1 mm×150 mm column, and separated over 60 minutes. XIC area counts for PAL tryptic peptides containing un-conjugated lysine were normalized to XIC area counts for a PAL tryptic peptide containing no conjugation sites (lysine) from the same sample. For a given peptide containing a lysine conjugation site, the normalized XIC area counts for a conjugated sample (PEG or POEGMA) is divided by the normalized XIC area for an unconjugated sample (rAvPAL). This value represents the % site occupancy for a given conjugation site, as shown in the equations below:
Peptide with Conjugation Site: HASK
Peptide without Conjugation Site (normalization peptide): FREEPEP
XIC Area Counts for HASK: [HASK]
XIC Area Counts for FREEPEP: [FREEPEP]
Subscripts for Samples:
45 kDa POEGMA-PAL: PEGMA
Pegvaliase: PEG
rAvPAL: PAL

$$\% PEGMA Site Occupancy for Peptide HASK = \frac{\frac{{[ HASK ]}_{PEGMA}}{{[ FREEPEP ]}_{PEGMA}}}{\frac{{[ HASK ]}_{PAL}}{{[ FREEPEP ]}_{PAL}}}$$

$$\% PEG Site Occupancy for Peptide HASK = \frac{\frac{{[ HASK ]}_{PEG}}{{[ FREEPEP ]}_{PEG}}}{\frac{{[ HASK ]}_{PAL}}{{[ FREEPEP ]}_{PAL}}}$$

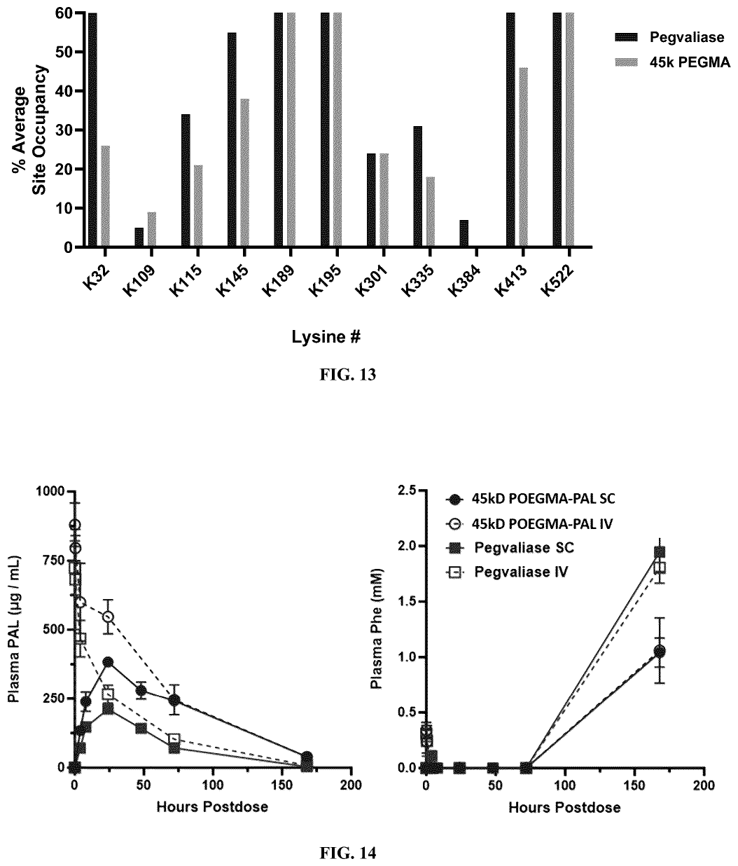

Results are shown in FIG. 13, where the x-axis lists the different lysine residues and the y-axis reports the average % occupancy.

### Example 7: Single Dose Study Evaluating Plasma PAL and Phenylalanine in Male ENU2 Mice Following IV and SC Doses of Compound 1-A and Pegvaliase

Male C₅₇BL6 PAH^{enu2} mice were administered subcutaneously (SC) or intravenously (IV) a single injection of 20 mg/kg (based on PAL) 45 kDa POEGMA-PAL or Pegvaliase. Blood samples were collected at 4 hours, 8 hours, 24 hours, 48 hours, 72 hours, and 168 hours post-dose for SC treated mice and 5 minutes, 30 minutes, 4 hours, 24 hours, 72 hours, and 168 hours for IV treated mice. These samples were then processed and analyzed as described in Example 5.

Results of the study are shown in FIG. 14.

The plasma PAL exposure is generally higher for Compound 1-A than Pegvaliase after IV and SC single doses at all timepoints of sampling. The PAL concentration-time profiles of Pegvaliase and Compound 1-A after IV and SC administrations exhibited a monophasic elimination profile. Plasma PAL and phenylalanine levels for both Compound 1-A and Pegvaliase showed an inverse relationship, with a decrease in phenylalanine observed as PAL levels increased. Independent of the route of administration, plasma phenylalanine levels for Pegvaliase generally returned to baseline (approximately 2 mM) by 168 hours at which point PAL levels were below quantification limit (BQL). In contrast, phenylalanine levels in mice treated with IV or SC Compound 1-A returned to approximately 1 mM with PAL levels remaining slightly higher at 168 hours in comparison with Pegvaliase.

The summary of pharmacokinetic parameters calculated non-compartmentally are shown in Table 1. Following a single IV administration of 20 mg/kg in ENU2 mice, Compound 1-A showed a slightly higher Cₘₐₓ (1.1-fold) compared to Pegvaliase. Similarly, the half-life of Compound 1-A was observed to be 1.1-fold longer than Pegvaliase. The clearance of PAL from plasma after Compound 1-A is slower than Pegvaliase. The 2.0-fold higher AUC_{inf} for Compound 1-A after single IV dose indicates a greater overall exposure in ENU2 mice than Pegvaliase.

Following single SC administration of 20 mg/kg in ENU2 mice, while the absorption rate to peak PAL plasma concentration was similar for Compound 1-A and Pegvaliase, the Cₘₐₓ was observed to be 1.8-fold higher for Compound 1-A over Pegvaliase. The overall exposure (AUC_{inf}) was 2.6-fold higher for Compound 1-A over Pegvaliase. Additionally, Compound 1-A exhibits a much slower PAL plasma clearance (0.39-fold) than Pegvaliase, resulting in a 1.54-fold longer terminal plasma half-life of PAL. Compound 1-A showed improved bioavailability of 73.2% following SC dosing compared to 57.6% for Pegvaliase.

In summary, after both IV and SC single doses, Compound 1-A exhibited an overall higher exposure (Cₘₐₓ, AUC) and longer terminal half-life over Pegvaliase, demonstrating improved pharmacokinetics in ENU2 mice model of PKU. Moreover, Compound 1-A demonstrated a superior pharmacodynamic effect (phenylalanine lowering) at the 168 hours timepoint in comparison with Pegvaliase.

TABLE 1
Summary of Plasma PK Parameters for Compound 1-A and Pegvaliase
Following a Single 20 mg/kg SC and IV injection in Male ENU2 Mice

| | | | | | AUCₗₐₛₜ | AUC_{inf} | Vz or | CL or | | |
|---|---|---|---|---|---|---|---|---|---|---|
| | Dose | | Tₘₐₓ^{a} | Cₘₐₓ | (h* | (h* | Vz/F^{b} | CL/F^{c} | T1/2 | |
| Group | (mg/kg) | | (h) | (μg/mL) | μg/mL) | μg/mL) | (ml/kg) | (mL/h/kg) | (h) | F % |
| Compound | 20 | N | 3 | 3 | 3 | 3 | 3 | 3 | 3 | 73.2 |
| 1-A | | Mean | 24 | 383 | 30800 | 33100 | 35.5 | 0.607 | 40.6 | |
| (SC) | | SD | 0 | 14.4 | 2430 | 2820 | 2.05 | 0.0504 | 1.3 | |
| Pegvaliase | 20 | N | 3 | 3 | 3 | 3 | 3 | 3 | 3 | 57.6 |
| (SC) | | Mean | 24 | 211 | 11700 | 12700 | 59.7 | 1.57 | 26.3 | |
| | | SD | 0 | 14 | 1470 | 740 | 4.14 | 0.0934 | 2.43 | |
| Compound | 20 | N | 3 | 3 | 3 | 3 | 3 | 3 | 3 | — |
| 1-A | | Mean | — | 886 | 43000 | 45200 | 23.7 | 0.452 | 36.9 | |
| (IV) | | SD | — | 71.8 | 7010 | 8290 | 1.19 | 0.0751 | 4.88 | |
| Pegvaliase | 20 | N | 3 | 3 | 3 | 3 | 3 | 3 | 3 | — |
| (IV) | | Mean | — | 810 | 20300 | 22200 | 42.5 | 0.902 | 32.7 | |
| | | SD | — | 51.7 | 2490 | 333 | 2.95 | 0.0135 | 2.7 | |

^{a}median values are reported for Tₘₐₓ

### Example 8: Single Dose PK/PD Study of Pegvaliase and Compound 1-A in Cynomolgus Monkeys

Cynomolgus monkeys were administered a single subcutaneous (SC) injection of 4 mg/kg (based on PAL) 45 kDa POEGMA-PAL or Pegvaliase. Blood samples were collected immediately after dosing (t=0) and at 3 hours, 6 hours, 9 hours, 12 hours, 18 hours, 24 hours, 30 hours, 36 hours, 48 hours, 60 hours, 72 hours, 84 hours, 96 hours, 120 hours, 144 hours, 168 hours, 192 hours, 216 hours, 264 hours, 312 hours, 360 hours, 432 hours, and 504 hours post-dose. These samples were then processed and analyzed similar to what is described in described in Example 5.

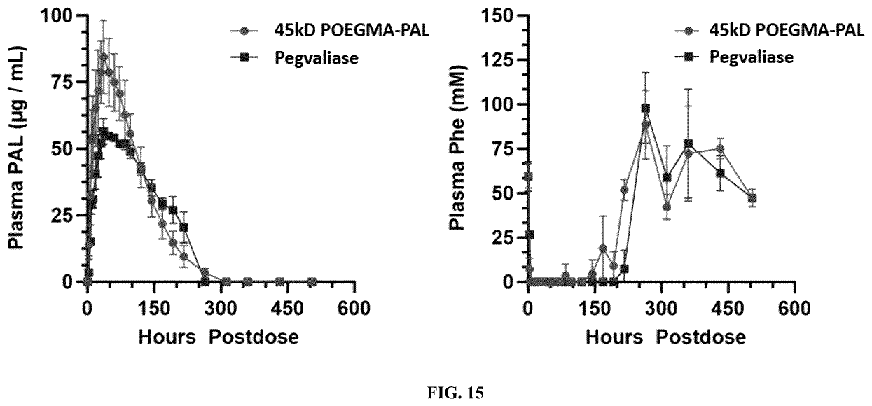

Results of the study are shown in FIG. 15.

Following a single SC administration of 20 mg/kg, the absorption of PAL is slightly slower for Pegvaliase with a median time to peak plasma PAL concentrations of 42 hours compared to 36 hours for Compound 1-A. The peak plasma concentrations of PAL for Compound 1-A were higher than Pegvaliase. Following the peak concentrations, Compound 1-A exhibited a rapid decline in PAL concentrations, while Pegvaliase pharmacokinetic profile suggested a longer absorption phase than Compound 1-A. The pharmacokinetic profiles of both Pegvaliase and Compound 1-A exhibited a monophasic decline of PAL concentrations. The Pegvaliase pharmacokinetic profile showed a sharp decline of PAL following 216 hours post dose, while the decline appeared natural for Compound 1-A. Plasma PAL concentrations were BLQ for Pegvaliase starting from 264 hours post dose, while for Compound 1-A, plasma PAL concentrations reached BLQ at 312 hours post dose. Plasma PAL curve generally showed an inverse relationship with plasma phenylalanine levels for both Pegvaliase and Compound 1-A in cynomolgus monkeys with a decrease in phenylalanine observed as PAL concentrations increased. Following single dose of Pegvaliase and Compound 1-A, phenylalanine levels dropped to BQL (<10 μM) in cynomolgus monkeys starting from 6 hours post dose and remained as BQL up to 216 hours in Pegvaliase group and up to 192 hours post dose in Compound 1-A group. As the PAL plasma concentrations reached BQL in Pegvaliase and Compound 1-A treated animals, the phenylalanine levels increased to near baseline values.

The summary of pharmacokinetic parameters calculated non-compartmentally are shown in Table 2. Following a single IV administration of 4 mg/kg in healthy cynomolgus monkeys, Compound 1-A showed a higher Cₘₐₓ (1.5-fold) compared to Pegvaliase. The overall exposure (AUC_{inf}) was slightly higher (1.1-fold) for Compound 1-A over Pegvaliase.

TABLE 2
Summary of Plasma PK Parameters for Pegvaliase and Compound 1-A Following
a Single 4 mg/kg SC administration in Male Cynomolgus Monkeys

| | Dose | | Tₘₐₓ | Cₘₐₓ | AUCₗₐₛₜ | AUC_{inf} | Vz/F | CL/F | T_{1/2} |
|---|---|---|---|---|---|---|---|---|---|
| Group | (mg/kg) | | (h) | (μg/mL) | (h*μg/mL) | (h*μg/mL) | (ml/kg) | (ml/h/kg) | (h) |
| Pegval- | 4 | N | 2 | 2 | 2 | 2 | 2 | 2 | 2 |
| iase | | Mean | 42 | 14.2 | 8895.3 | 9189.3 | 63.8 | 0.437 | 101.7 |
| | | SD | 8.48 | 1.04 | 710.4 | 736.6 | 0.586 | 0.035 | 9.08 |
| 1-A | 4 | N | 3 | 3 | 3 | 3 | 3 | 3 | 3 |
| | | Mean | 36 | 21.1 | 10107.3 | 10142.5 | 27.9 | 0.403 | 48.6 |
| | | SD | 0 | 3.45 | 1917.9 | 1921.5 | 2.29 | 0.072 | 5.07 |

^{a} median values are reported for Tₘₐₓ

In an ongoing study, 2 mL of Pegvaliase (3 mg/kg) or Compound 1-A (either 1 mg/kg PAL or 3 mg/kg PAL) is administered to cynomolgus monkeys twice weekly for 8 weeks.

Throughout this application, various publications, patents, patent applications and other documents have been referenced. The disclosures of these publications, patents, patent applications and other documents in their entireties are hereby incorporated by reference in this application for all purposes, including in order to more fully describe the state of the art to which this the subject matter disclosed herein pertains. Although the disclosed subject matter has been described with reference to the examples provided above, it should be understood that various modifications could be made without departing from the spirit of the disclosed subject matter. Many variations will become apparent to those skilled in the art upon review of this specification.

1. A conjugate comprising:
a. one or more polymers comprising poly(oligoethylene glycol methacrylate); and
b. phenylalanine ammonia lyase (PAL),
wherein the one or more polymers is directly or indirectly bound to the PAL via one or more lysine residues, wherein the conjugate is represented by Formula (I): 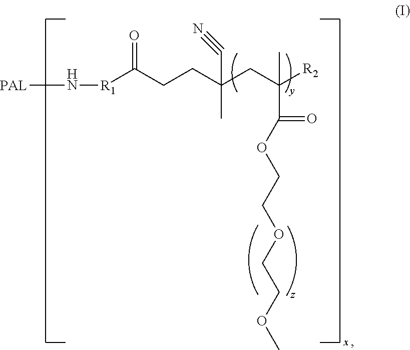
wherein:
PAL is the prokaryotic phenylalanine ammonia-lyase, which is a homotetrameric protein comprising four subunits;
R₁ is a linker;
R₂ is an end cap;
x is an integer from 1 to about 32;
y is an integer from about 50 to about 500;
z is an integer from 2 to about 10; and
the structure inside the square brackets together with subscript x represents the one or more polymers comprising poly(oligoethylene glycol methacrylate).

2. (canceled)

3. The conjugate of claim 1, wherein R₁ is C₁₋₁₂ alkyl, C(O)—C₁₋₁₂ alkyl-NH, C(O)—(O—CH₂—CH₂) n, wherein n is 2 or 3, or Gly-Gly-Phe-Gly (SEQ ID NO:5).

4. The conjugate of claim 1, wherein the conjugate is represented by Formula (I′): 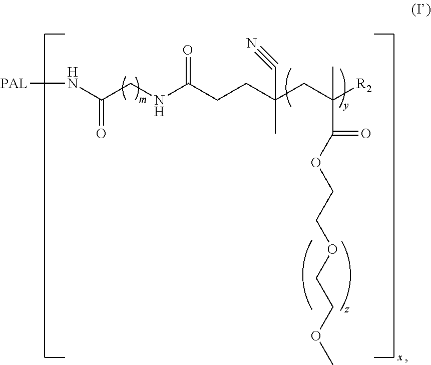
wherein:
PAL is the prokaryotic phenylalanine ammonia-lyase, which is a homotetrameric protein comprising four subunits;
R₂ is an end cap;
m is an integer from 3 to 12;
x is an integer from 1 to about 32;
y is an integer from about 50 to about 500; and
z is an integer from 2 to about 10.

5. The conjugate of claim 4, wherein x is an integer from about 16 to about 28.

6. The conjugate of claim 4, wherein at least five lysine residues on each PAL subunit are bound to at least five of the polymers comprising poly(oligoethylene glycol methacrylate).

7. (canceled)

8. The conjugate of claim 1, wherein R₂ is isobutyronitrile, 4-cyanovaleric acid, 4-methoxy-2,4-dimethylvaleronitrile, and hydroxy (—OH).

9. (canceled)

10. The conjugate of claim 1, wherein z is an integer from 2 to about 5.

11-14. (canceled)

15. The conjugate of claim 1, wherein y is an integer from about 150 to about 300.

16. The conjugate of claim 1, wherein x is an integer from about 16 to about 28, y is an integer from about 175 to about 200, and z is 2 or 3.

17-18. (canceled)

19. The conjugate of claim 1, wherein the conjugate of Formula (I) is a conjugate of Formula (Ia-1-1):

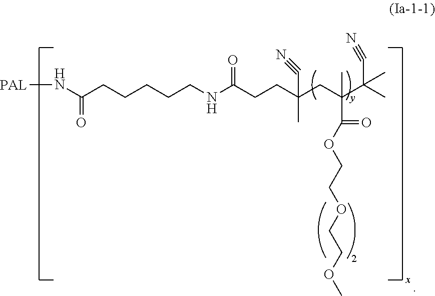

20. (canceled)

21. The conjugate of claim 1, wherein the PAL is derived from Anabaena variabilis (AvPAL), wherein each subunit of the AvPAL comprises an amino acid sequence of SEQ ID NO:1, SEQ ID NO:2, SEQ ID NO: 3, or SEQ ID NO:4.

22-30. (canceled)

31. A composition comprising the conjugate of claim 1.

32-42. (canceled)

43. A pharmaceutical composition comprising the conjugate of claim 1 and a pharmaceutically acceptable excipient.

44. The pharmaceutical composition of claim 43, wherein the conjugate of is present in the composition at a concentration of about 20 mg/mL to about 60 mg/mL.

45. (canceled)

46. A kit comprising a syringe comprising the pharmaceutical composition of claim 43.

47. (canceled)

48. A method of making a conjugate of Formula (I): 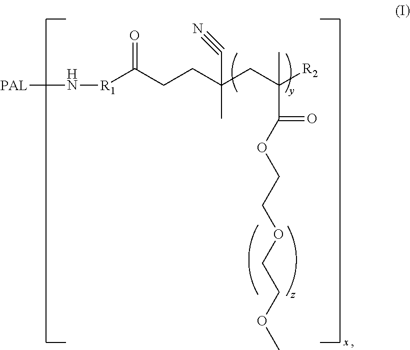
wherein:
PAL is a prokaryotic phenylalanine ammonia-lyase;
R₁ is a linker;
R₂ is an end cap;
x is an integer from 1 to about 32;
y is an integer from about 50 to about 500; and
z is an integer from 2 to about 10.
the method comprising:
a. contacting oligoethylene glycol methacrylate with a compound of Formula (II): 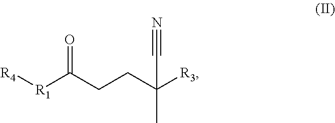
wherein R₃ is a RAFT chain transfer agent and R₄ is a conjugation moiety, under conditions effective to yield a polymer of Formula (III): 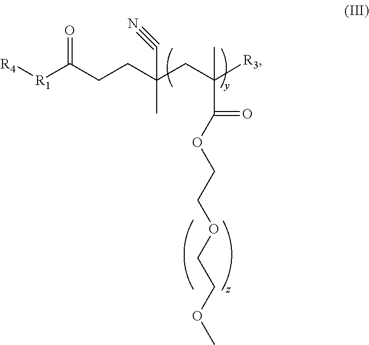
b. contacting the polymer of Formula (III) with an end group modification agent to remove R₃ and generate a pre-conjugation polymer of Formula (IV): 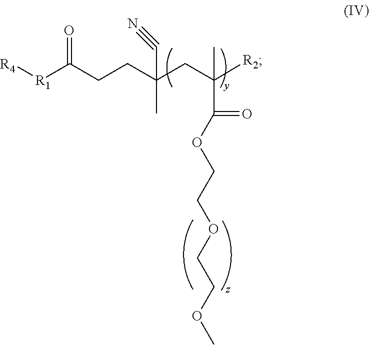
wherein R₂ is an end cap; and
c. contacting the pre-conjugation polymer of Formula (IV) with one or more lysine residues on PAL to generate the POEGMA-PAL conjugate of Formula (I).

49-65. (canceled)

66. A method of treating phenylalanine hydroxylase deficiency (PAH) in a subject comprising administering the pharmaceutical composition of claim 43 to the subject.

67. A method of treating phenylketonuria (PKU) in a subject comprising administering the pharmaceutical composition of claim 43 to the subject.

68. A method of reducing phenylalanine blood levels in a subject comprising administering the pharmaceutical composition of claim 43 to the subject.

69. The method of claim 66, wherein the administering is by subcutaneous injection.

70-75. (canceled)
